(19) United States
(12) Patent Application Publication
(10) Pub. No.: US 2026/0120868 A1
(43) Pub. Date: Apr. 30, 2026

(54) SYSTEMS AND METHODS OF USING MULTIPLE MODALITIES OF DATA WITH MACHINE-LEARNING MODELS

(71) Applicant: Tempus AI, Inc., Chicago, IL (US)

(72) Inventors: Erik T. Mueller, Chevy Chase, MD (US); Roosheel Patel, New York, NY (US); Raphael Pelossof, New York, NY (US); Alberto Purpura, Astoria, NY (US); Abigail Michelle Lammers, Chicago, IL (US)

(21) Appl. No.: 19/004,742

(22) Filed: Dec. 30, 2024

Related U.S. Application Data
(60) Provisional application No. 63/712,334, filed on Oct. 25, 2024.

(51) Int. Cl. G16H 50/20; G06F 16/34; G16H 10/60

(52) U.S. Cl. CPC G16H 50/20; G06F 16/345; G16H 10/60

(57) ABSTRACT

This application describes, amongst other things, an example method for responding to user queries. The method includes obtaining a set of data items comprising a plurality of modalities, and generating, using one or more ML models, summary data for the set of data items. The summary data includes a first type of summary data for the first plurality of data items, and a second type of summary data for the second plurality of data items. The method also includes generating a set of multi-modal embeddings using the first type and second types of summary data; and providing the set of multi-modal embeddings to a multi-modal ML model. The method further includes providing information from a user request to the multi-modal ML model; and receiving an output from the multi-modal ML model that is based on the information from the user request and the set of multi-modal embeddings.

### PRIORITY AND RELATED APPLICATIONS

This application claims priority under 35 U.S.C. § 119 from U.S. Provisional Application No. 63/712,334 filed on Oct. 25, 2024, entitled “Systems and Methods of Structuring and Querying Subject Data,” which is hereby incorporated by reference in its entirety.

### TECHNICAL FIELD

The disclosed embodiments relate to multi-modal machine learning architectures, including but not limited to, generating and using summaries and embeddings of multiple modalities of data to generate responses to user queries and prompts.

### BACKGROUND

Many professions require complex thought where people need to consider many factors when selecting solutions to encountered situations, hypothesize new factors and solutions, and test new factors and solutions to ensure that they are effective. For instance, oncologists considering specific patient cancer states, optimally should consider many different factors when assessing the patient's cancer state as well as many other factors when crafting and administering an optimized treatment plan.

Recently, machine learning (ML) has advanced to the point where it can assist professional in making educated decisions by uncovering patterns and insights from complex datasets, enabling predictive and prescriptive analytics. By processing vast amounts of data quickly and accurately, ML models can provide recommendations, identify trends, and support strategic planning in fields such as healthcare, finance, and logistics. However, for the ML outputs to be fully informed, the ML systems need access to a diverse modality of data, such as text, images, audio, and test data. For example, a patient's health record may include x-ray images, ultrasound images, biological sequencing data, unstructured text notes, structured patient data, and/or other data modalities. Therefore, the ML systems need to be configured to process multiple data modalities so as to have comprehensive, high-quality datasets. Otherwise, the ML models may make incomplete and/or biased predictions.

### SUMMARY

Thus, the inventors of the present application recognized a need for systems and methods that summarize and generate embeddings (e.g., tokenizations) for different modalities of data to integrate the diverse information into a unified representation. In this way multiple modalities may be input into a multi-modal ML model and the multi-modal ML model can provide well-informed outputs. For example, natural language processing models can distill textual content, computer vision models can extract features from images, and biological models can provide labels of biological test data. Each of these models may generate an individual summary or embedding, and the individual summaries and embeddings may be combined into multi-modal embeddings that are input into the multi-modal ML model. This approach allows the ML model to draw insights from a comprehensive dataset, thereby providing more accurate predictions and/or recommendations.

Among other things, the present disclosure provides systems and methods for generating inferences from multi-modal data. For example, a set of one or more agents is configured to operate as a digital specialist configured to transform different data modalities (e.g., structured data, unstructured data, genomic data, radiology data, pathology data, cardiology data, endocrinology data, mental health data, and the like) into a set of embeddings (e.g., transformer embeddings, vectorized tokenizations, and/or textual representations). Thus, the digital specialist may transform data and/or features (e.g., specific attributes extracted from raw data) that correspond to one or more data modalities. The digital specialist may be configured (e.g., according to a set of templates) to summarize, predict, or otherwise digitize precision medicine from a variety of sources (and potentially in a variety of formats). The digital specialist may respond to a query or request using one or more data modalities (e.g., based on the individual query/request).

In accordance with some embodiments, a method of generating inferences from multi-modal data includes: (i) obtaining a set of data items comprising a plurality of modalities, the set of data items including a first plurality of data items of a first modality and a second plurality of data items of a second modality; (ii) generating, using one or more ML models, summary data for the set of data items, the summary data including: (a) a first type of summary data for the first plurality of data items, and (b) a second type of summary data for the second plurality of data items; (iii) generating a set of multi-modal embeddings using the first type of summary data and the second type of summary data; (iv) providing the set of multi-modal embeddings to a multi-modal ML model, the multi-modal ML model being distinct from the one more ML models; (v) providing information from a user request to the multi-modal ML model; (vi) receiving an output from the multi-modal ML model that is based on the information from the user request and the set of multi-modal embeddings; and (vii) generating a response for the user using the output from the multi-modal ML model. As described in greater detail below, in various embodiments, the method includes a subset, or superset, of the actions listed above.

In accordance with some embodiments, a computing system is provided, such as a cloud computing system, a server system, a personal computer system, and/or other type of electronic device. The computing system includes control circuitry and memory storing one or more sets of instructions. The one or more sets of instructions include instructions for performing any of the methods described herein.

In accordance with some embodiments, a non-transitory computer-readable storage medium is provided. The non-transitory computer-readable storage medium stores one or more sets of instructions for execution by a computing system. The one or more sets of instructions include instructions for performing any of the methods described herein.

Thus, devices and systems are disclosed with methods for importing, structuring, and/or analyzing data. Such methods, devices, and systems may complement or replace conventional methods, devices, and systems for importing, structuring, and/or analyzing data.

The features and advantages described in the specification are not necessarily all inclusive and, in particular, some additional features and advantages will be apparent to one of ordinary skill in the art in view of the drawings, specification, and claims provided in this disclosure. Moreover, it should be noted that the language used in the specification has been principally selected for readability and instructional purposes and has not necessarily been selected to delineate or circumscribe the subject matter described herein.

### BRIEF DESCRIPTION OF THE DRAWINGS

So that the present disclosure can be understood in greater detail, a more particular description can be had by reference to the features of various embodiments, some of which are illustrated in the appended drawings. The appended drawings, however, merely illustrate pertinent features of the present disclosure and are therefore not necessarily to be considered limiting, for the description can admit to other effective features as the person of skill in this art will appreciate upon reading this disclosure.

FIG. 1 is a block diagram illustrating an example platform in accordance with some embodiments.

FIGS. 2A and 2B are block diagrams illustrating an example client device in accordance with some embodiments.

FIG. 2C illustrates examples of various logic functions that are implemented in accordance with some embodiments.

FIG. 3A is a block diagram illustrating an example server system in accordance with some embodiments.

FIG. 3B is a block diagram illustrating example databases in accordance with some embodiments.

FIG. 4 illustrates an example architecture for deploying agents in accordance with some embodiments.

FIG. 5A illustrates an example process for data importation and query processing in accordance with some embodiments.

FIG. 5B illustrates an example workflow for interacting with an agent in accordance with some embodiments.

FIG. 6 illustrates an example process for using multi-modal data in an agent system in accordance with some embodiments.

FIG. 7 illustrates an example process for using multi-modal data with missing modalities in accordance with some embodiments.

FIG. 8A illustrates an example process for identifying important modalities from a multi-modal analysis in accordance with some embodiments.

FIG. 8B illustrates another example process for identifying important modalities from a multi-modal analysis in accordance with some embodiments.

FIG. 9 illustrates an example process for applying type-specific criteria to model outputs in accordance with some embodiments.

FIGS. 10A-10J illustrate example user interfaces and interactions for importing and querying subject data in accordance with some embodiments.

FIG. 11A illustrates an example user interface for agent construction in accordance with some embodiments.

FIG. 11B illustrates an example task-specific orchestration for cell annotation in accordance with some embodiments.

FIGS. 11C-11D illustrate an example cell annotation in accordance with some embodiments.

FIG. 12 illustrates an example architecture for slide summarization in accordance with some embodiments.

FIGS. 13A-13B illustrate an example architecture and procedure for generating inferences on survivorship in accordance with some embodiments.

FIG. 14 is a flow diagram illustrating an example method of generating inferences from multi-modal data in accordance with some embodiments.

In accordance with common practice, the various features illustrated in the drawings are not necessarily drawn to scale, and like reference numerals can be used to denote like features throughout the specification and figures.

### DETAILED DESCRIPTION

The present disclosure describes, among other things, a platform for using task-specific orchestrations (e.g., task-specific agents) that include task-specific machine-learning models (e.g., language models, transformer models, diffusion models, and other types of models) for specific tasks and/or within specific domains as well as multi-modal models for tasks involving multiple modalities of data. The platform may include a plurality of individual task-specific orchestrations that may operate independently or in combination to return accurate and relevant information (e.g., identifying target cohorts, clinical trial information, and/or members of target populations). In some embodiments, the platform includes a plurality of modality-specific orchestrations (e.g., each configured to summarize a corresponding modality of data) and one or more multi-modal orchestration (e.g., configured to intake and analyze multiple modalities of data). In some embodiments, each orchestration (or agent) may include one or more machine-learning models, such as a language model trained and/or fine-tuned on a particular domain. The platform may also include one or more composite orchestrations (e.g., composite agents) that give instructions to, and combine results from, a plurality of task-specific orchestrations configured for different tasks.

In some embodiments, the platform acts as an operating system for implementing orchestrations to perform various clinical tasks. The platform may include one or more of the following example components. For example, a genetic sequencing component with downstream molecular bioinformatics may operate to call out relevant biomarkers in DNA, RNA, or their derivatives for a specimen (e.g., a tumor biopsy) that is sequenced and reported back to an ordering physician. As another example, a pathology imaging component may operate on cellular and/or slide level images to identify relevant biomarkers from cells within imaged specimen. As another example, a radiological imaging component may operate on larger images of the body through various radiology imaging technologies to identify the presence or longitudinal progression of tumors. Other examples include identifying various disease states using cardiology, neurology, and/or endocrinology imaging components. Each of these components may include, or communicate with, a corresponding agent to identify and/or report information relevant to a user query or request.

As an example, an orchestration (agent) may be configured by a user using a user interface (e.g., a console of a web or desktop application) and deployed to various environments (e.g., a research environment, an alpha environment, a beta environment, a client environment, and/or a production environment). Each environment may be linked to different sources, have different permissions, and/or have different authorized users. In some embodiments, precision medicine principles are employed in customizing the user interfaces, such as modifications based on a set of subjects (e.g., patients) associated with the user of the application. For example, the user (or an immediate family member of the user) may be one of the subjects. An environment may be defined by access to data sources and/or users. The agent configuration may be stored in a control plane. The control plane may be configured to control how data is managed, routed, and/or processed. The agents themselves may execute in the appropriate workload planes (e.g., data planes), and the workload planes may not have access to the control plane. The control plane may supervise/direct each workload plane, while the workload planes are configured to manipulate and/or transport data.

As an example, an agent builder in the control plane may be configured to push configurations into the various environments. For example, this synchronization may be fast enough that a user can configure an agent and immediately evaluate the configuration in the interactive console in a working environment. An example architecture includes two components: an agent builder in a control plane that hosts the user interface (UI) for configuring agents, and an agent host in a workload plane that hosts the UI and API for interacting with deployed agents. When an agent configuration is changed or an agent version is deployed, the agent builder may inform the agent host in each environment so that the updated agent can be deployed. For example, this may be via a pubsub message to the agent-config topic or via a simple HTTP request. In some embodiments, the agent builder utilizes a cognitive architecture that includes memory modules and action spaces. For example, the cognitive architecture organizes agents along three dimensions: their information storage (e.g., divided into working and long-term memories); their action space (e.g., divided into internal and external actions); and their decision-making procedure (e.g., structured as an interactive loop with planning and execution).

As another example, after deployment, an agent may receive a user query (e.g., requesting information about clinical trials), generate a structured application programming interface (API) call, use the generated API call to query a remote server to retrieve a relevant result, and reformat the relevant information to return to the user. In some embodiments, each action is performed by a different agent builder block component (also sometimes referred to as a builder block, block, or node). In some embodiments, the agent is configured for multiple types of tasks. In these embodiments, the agent may identify the intent of a user's query (e.g., to search for clinical trials or identify adverse events) and respond accordingly. In some embodiments, the agent is configured for only one type of task (e.g., is a task-specific agent). In some of these embodiments, the agent does not identify an intent of the user (e.g., the agent may assume the intent). In some embodiments, the agent receives the intent from a different component or system. The agent may also interface with other agents to obtain additional information for the user query (such as patient records or relevant guidelines). In some embodiments, the agent includes a pretrained language model (e.g., trained on a particular domain and/or using particular databases). In some embodiments, the agent queries an unstructured database (e.g., in addition, or alternatively, to generating the API call).

The platform, or components thereof, may be used in conjunction with any medical field (e.g., to assist physicians in the treatment of any associated disease state therein), such as on oncology, endocrinology (e.g., diabetes), neurology, mental health (e.g., depression and related pharmacogenetics), and cardiovascular disease. For example, the platform may also include a cardiology-based component (e.g., comprising one or more agents) that operates on electrocardiogram (ECG) data to identify patients having an elevated risk for cardiovascular disease. As another example, the platform may include a data curation component (e.g., comprising one or more agents) that obtains raw (e.g., unstructured) data and structures it into a common and useful format as a repository (e.g., a multimodal database) of clinical data from which other bioinformatics, analytics, agents, models, and/or components may operate. As another example, the platform may be configured to search within the clinical data to identify cohorts of related patients and/or generate insights and/or analytics. As another example, the platform may be configured to monitor an electronic health record (EHR) to identify care gaps and/or reminders to physicians to act with a respective patient. In this way, the platform may serve as a docket manager that identifies issues/events the corresponding physicians did not manually docket, e.g., to ensure patients and other subjects get timely care. The platform may also be configured to track and/or catalog relevant therapies (e.g., on label and/or off label use) for a set of disease states. The platform may also track and/or catalog relevant clinical trials (e.g., in multiple countries and/or from multiple authorities) for a set of disease states. In some embodiments, the platform is further configured to interact with patients/subjects directly.

As discussed below, the platform may include an AI-enabled assistive user interface (which may sometimes be described herein as a clinical assistant or digital assistant) that provides access to patient insights. The AI-enabled assistive user interface may use one or more of the orchestrations described herein, each of which may include ML models and/or other types of machine learning.

In some embodiments, the platform includes a hub component that allows physicians to order, track, and view test results, and export patient data. In some embodiments the hub component provides insights into genomic alterations, treatment implications, as well as clinical trial matching. The hub component may be used in conjunction with the AI-enabled clinical assistant to allow physicians to interact using conversational language including natural language inputs, follow-up questions, and remarks. The platform may also include a peer-to-peer messaging component for physicians and other medical experts to share knowledge, insight, and/or perspective on medical fields such as molecular oncology (e.g., as it pertains to patient care). The messaging component may be used in conjunction with the AI-enabled clinical assistant to engage in, and optionally learn from, the conversations on the messaging component. For example, the AI-enabled clinical assistant may be invoked in conversation to provide insights and/or data for a particular topic or conversation. The platform may also include an EHR interface component (e.g., comprising one or more agents) configured to allow physicians, and optionally other users, to view, edit, and/search an EHR. The EHR interface component may be communicatively coupled with one or more services and/or databases to obtain updated information and reports (e.g., via push notifications). The EHR interface component may be used in conjunction with the AI-enabled clinical assistant to search, edit, summarize, and/or reform an EHR. The platform may also include a research analytical component (e.g., comprising one or more agents) that provides de-identified patient/clinical data and insights. For example, the platform may provide insights derived from providing available data and/or newly-ingested data to a machine-learning model (e.g., the insights are output by the model in response to providing the data).

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

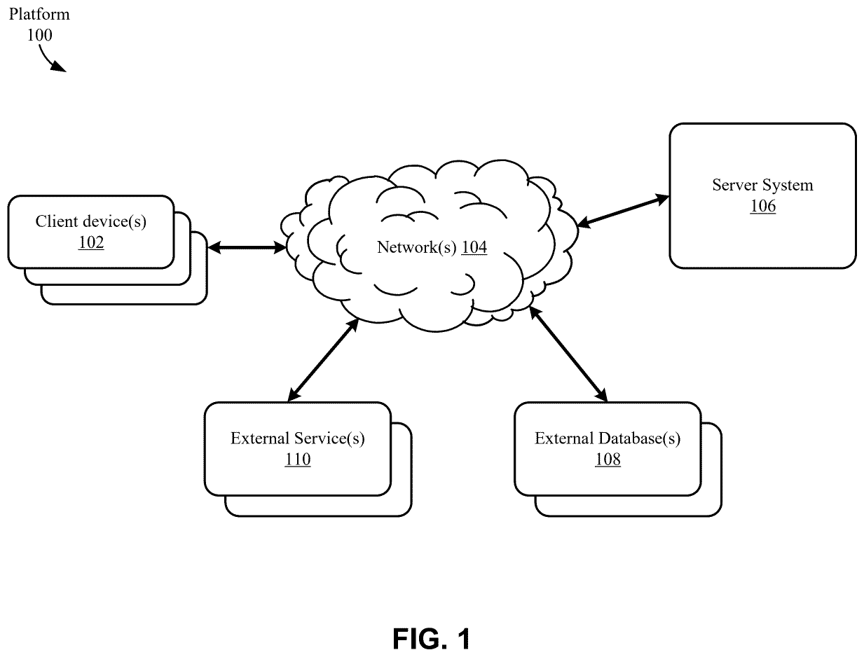

FIG. 1 is a block diagram illustrating a platform 100 in accordance with some embodiments. In some embodiments, the platform 100 is an AI platform (e.g., the AI platform discussed previously). The platform 100 includes one or more client devices 102 communicatively coupled to a server system 106 via one or more networks 104. In accordance with some embodiments, the platform 100 further includes, or communicates with, one or more external services 110 and one or more external databases 108. In some embodiments, the one or more networks 104 include public communication networks, private communication networks, or a combination of both public and private communication networks. For example, the one or more networks 104 can be any network (or combination of networks) such as the Internet, other wide area networks (WAN), local area networks (LAN), virtual private networks (VPN), metropolitan area networks (MAN), peer-to-peer networks, and/or ad-hoc connections. In some embodiments, the platform 100 includes only a subset of the components shown in FIG. 1. For example, the platform 100 may include only one of: a client device 102 or a server system 106.

In some embodiments, a client device 102 is associated with one or more users. In some embodiments, each user is separately authenticated (e.g., assigned distinct/unique authentication tokens). In some embodiments, a client device 102 is a personal computer, mobile electronic device, wearable computing device, laptop computer, tablet computer, mobile phone, feature phone, smart phone, a speaker, television (TV), and/or any other electronic device capable of interacting with a user (e.g., an electronic device having an I/O interface). The client device(s) 102 may communicatively couple to other components of the platform 100 wirelessly and/or through a wired connection (e.g., directly through an interface, such as an HDMI interface).

In some embodiments, the client device(s) 102 send and receive information, such as documents, queries, and/or results, through network(s) 104. For example, the client device(s) 102 may send a query or request to the server system 106, the external service(s) 110, and/or the external database(s) 108 through network(s) 104. As another example, the client device(s) 102 may receive results and other responses from the server system 106, the external service(s) 110, and/or the external database(s) 108 through network(s) 104. In some embodiments, two or more client devices 102 communicate with one another (e.g., resending and responding to queries and requests). The two or more client devices 102 may communicate via the network(s) 104 or directly (e.g., via a wired connection or through a peer-to-peer wireless connection).

In some embodiments, the server system 106 includes multiple electronic devices communicatively coupled to one another. In some embodiments, the multiple electronic devices are collocated (e.g., in a datacenter), while in other embodiments, the multiple electronic devices are geographically separated from one another. In some embodiments, the server system 106 stores and provides clinical and/or patient data. In some embodiments, the server system 106 trains, publishes, and/or utilities one or more agents and/or language models. In some embodiments, the server system 106 receives and responds to queries and requests from the client device(s) 102 using the one or more agents and/or language models. In some embodiments, the server system 106 includes multiple nodes and/or clusters configured to manage different types of tasks and/or handle requests and queries from different geographical locations.

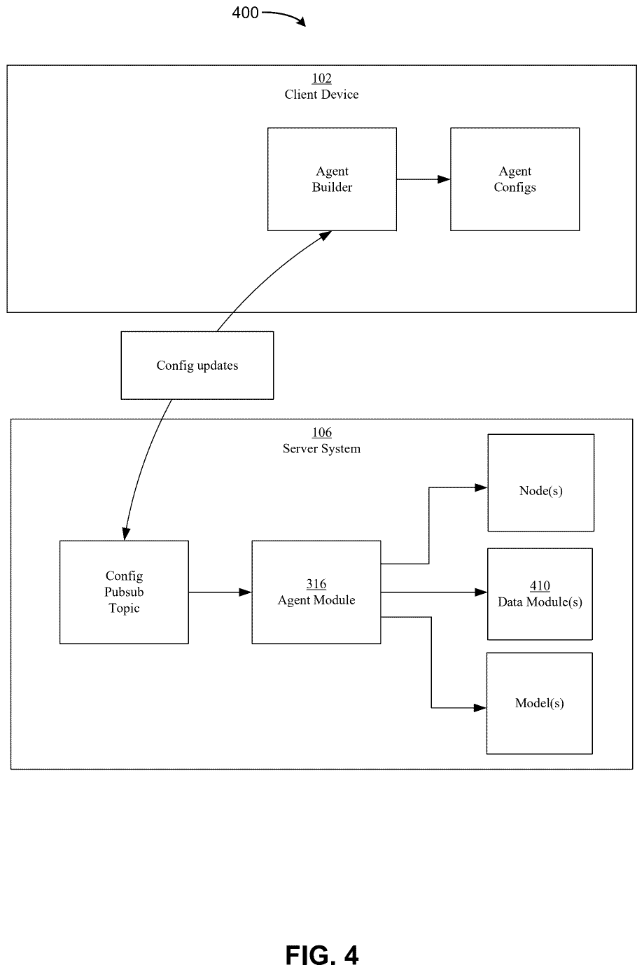

In some embodiments, the client device(s) 102 and/or the server system 106 communicate with the external service(s) 110 and/or the external database(s) 108 via an application programming interface (API). In some embodiments, the external service(s) 110 and/or the external database(s) 108 are maintained/operated by a third party to the platform 100. In some embodiments, the external service(s) 110 include agents, location services, time services, web-enabled services, and/or services that access information stored external to the platform 100. In some embodiments, the external database(s) 108 include one or more medical databases, clinical databases, subject databases, research databases, and/or general knowledge databases. In some embodiments, the external database(s) 108 comprise one or more of the databases shown in FIG. 4. In some embodiments, the external database(s) 108 comprise one or more user databases (e.g., patient databases maintained by a third-party user of the platform 100).

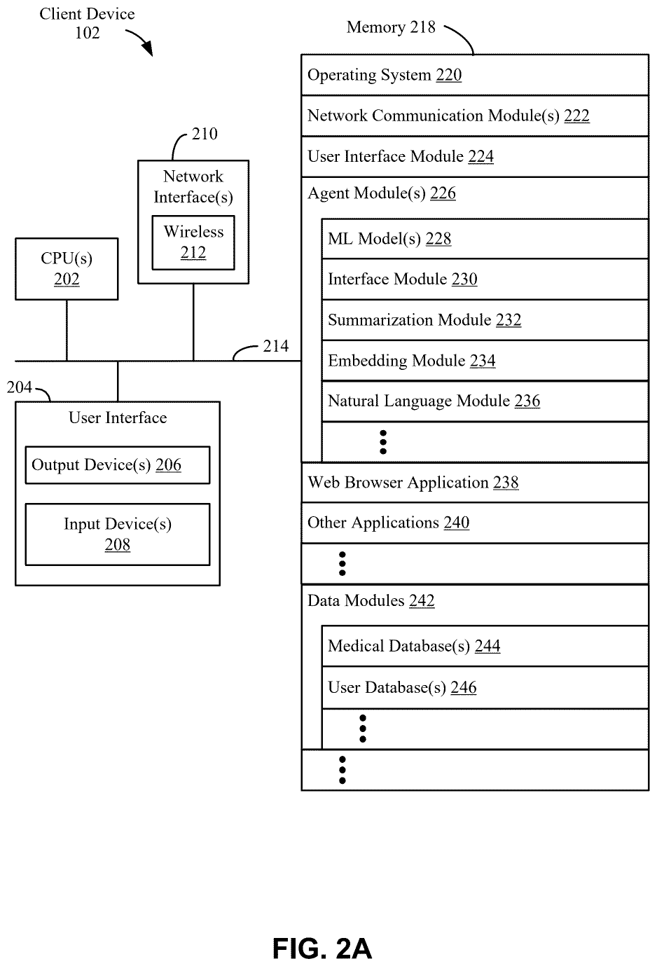

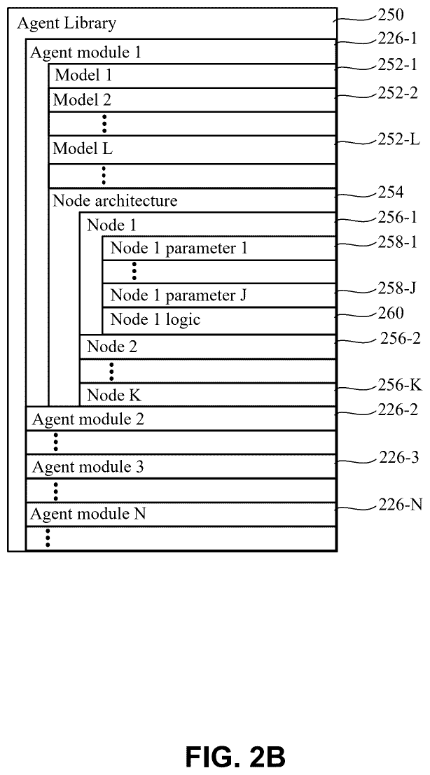

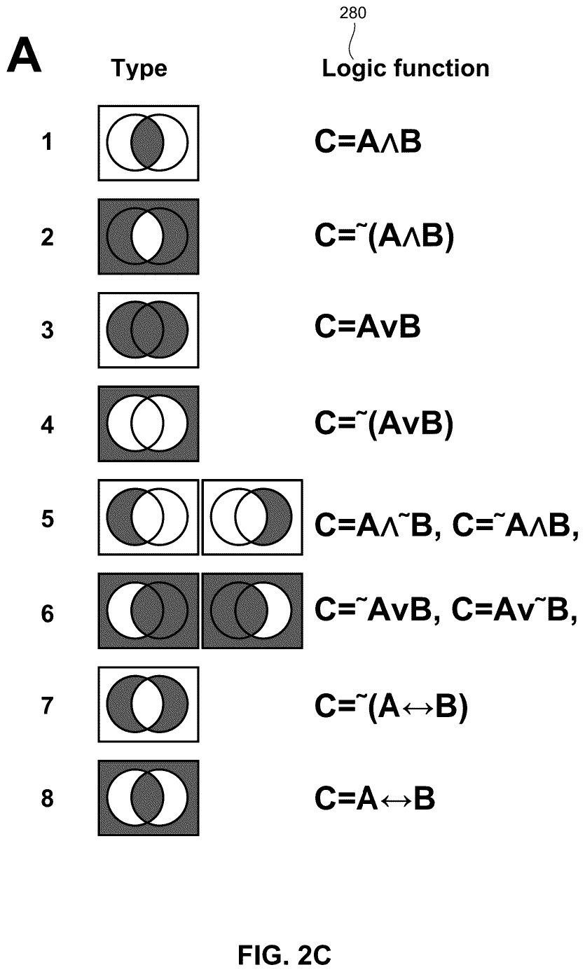

FIG. 2A is a block diagram illustrating a client device 102 in accordance with some embodiments. The client device 102 includes one or more central processing units (CPUs) 202, a user interface 204, one or more network (or other communications) interfaces 210, memory 218, and one or more communication buses 214 for interconnecting these components. In some embodiments, the client device 102 includes a processor or other control circuitry (e.g., in addition, or alternatively, to the CPUs 202). For example, the client device 102 may include one or more GPUs and/or DPUs (e.g., for performing machine learning tasks). The communication buses 214 optionally include circuitry (sometimes called a chipset) that interconnects and controls communications between system components. Optionally, the client device 102 includes a location-detection component, such as a global navigation satellite system (GNSS) (e.g., GPS (global positioning system), GLONASS, Galileo, BeiDou) or other geo-location receiver, and/or location-detection software for determining the location of the client device 102.

In some embodiments, the client device 102 includes one or more sensors including, but not limited to, accelerometers, gyroscopes, compasses, magnetometer, light sensors, near field communication transceivers, barometers, humidity sensors, temperature sensors, proximity sensors, range finders, and/or other sensors/devices for sensing and measuring various environmental conditions.

The user interface 204 includes output device(s) 206 and input device(s) 208. In some embodiments, the input device(s) 208 include a keyboard, mouse, a track pad, and/or a touchscreen. In some embodiments, the user interface 204 includes a display device that includes a touch-sensitive surface, in which case the display device is a touch-sensitive display. In client devices that have a touch-sensitive display, a physical keyboard is optional (e.g., a soft keyboard may be displayed when keyboard entry is needed). In some embodiments, the output device(s) 206 include a speaker and/or a connection port for connecting to speakers, earphones, headphones, or other external listening devices. In some embodiments, the input device(s) 208 include a microphone and/or voice recognition device to capture audio (e.g., speech from a user).

In some embodiments, the one or more network interfaces 210 include wireless and/or wired interfaces for receiving data from and/or transmitting data to other client devices 102, the server system 106, and/or other devices or systems. The data communications may be conducted using any of a variety of custom or standard wireless protocols (e.g., NFC, RFID, IEEE 802.15.4, Wi-Fi, ZigBee, 6LoWPAN, Thread, Z-Wave, Bluetooth, ISA100.11a, WirelessHART, MiWi, etc.). Furthermore, the data communications may be conducted using any of a variety of custom or standard wired protocols (e.g., USB, Firewire, Ethernet, etc.). For example, the one or more network interfaces 210 may include a wireless interface 212 for enabling wireless data communications with other client devices 102, systems, and/or other wireless (e.g., Bluetooth-compatible) devices. Furthermore, in some embodiments, the wireless interface 212 (or a different communications interface of the one or more network interfaces 210) enables data communications with other WLAN-compatible devices and/or the server system 106 (via the one or more network(s) 104).

The memory 218 includes high-speed random-access memory, such as DRAM, SRAM, DDR RAM, or other random-access solid-state memory devices; and may include non-volatile memory, such as one or more magnetic disk storage devices, optical disk storage devices, flash memory devices, or other non-volatile solid-state storage devices. The memory 218 optionally includes one or more storage devices remotely located from the CPU(s) 202. The memory 218, or alternately, the non-volatile memory solid-state storage devices within the memory 218, includes a non-transitory computer-readable storage medium. In some embodiments, the memory 218 or the non-transitory computer-readable storage medium of the memory 218 stores the following programs, modules, and data structures, or a subset or superset thereof:
an operating system 220 that includes procedures for handling various basic system services and for performing hardware-dependent tasks;
network communication module(s) 222 for connecting the client device 102 to other computing devices connected to one or more network(s) 104 via the one or more network interface(s) 210 (wired or wireless);
a user interface module 224 that receives commands and/or inputs from a user via the user interface 204 (e.g., from the input device(s) 208) and provides outputs via the user interface 204 (e.g., the output device(s) 206);
agent module(s) 226 that include a set of agent building blocks and/or generated agents. In some embodiments, the agent module(s) 226 work in conjunction with an agent module at the server system 106 (e.g., the agent module(s) 316). In some embodiments, the agent module(s) 226 includes the following submodules (or sets of instructions), or a subset or superset thereof:
   model(s) 228 that engage with a user and/or perform specific tasks (e.g., in furtherance of a user request or query). In some embodiments, the model(s) 228 include one or more large language models, such as GPT-3, GPT-4, BioGPT, and PaLM-2, neural networks, transformer models, and/or other types of ML models; and
   an interface module 230 that allows the model(s) 228 to communicate with other applications, components, and devices (e.g., via an API or structured query). In some embodiments, the interface module 230 is, or includes, an agent (e.g., a task-specific orchestration, a modality-specific orchestration, or a multi-modal orchestration), an orchestration creator application, one or more orchestration libraries (e.g., orchestration marketplaces) for selecting orchestrations for performing tasks as discussed herein;
   a summarization module 232 that is configured to summarize one or more modalities of data, such as summarizing of a medical visit, annotating and/or labeling images, and/or otherwise summarizing data (e.g., in a human-readable format, such as a natural language summary);
   an embedding module 234 that is configured to generate embeddings (e.g., vectors) based on input data, such as raw input data and/or summarized input data. In some embodiments, the embedding module 234 is configured to generate modality-specific embeddings. In some embodiments, the embedding module 234 is configured to generate multi-modal embeddings (e.g., by aggregating or combining modality-specific embeddings);
   a natural language module 236 that is configured to generate natural language (e.g., conversational) outputs. In some embodiments, the natural language module 236 is configured to convert one or more ML outputs into a natural language output. In some embodiments, the natural language module 236 is configured to generate embeddings from natural language inputs (e.g., received from a user via a digital assistant interface);
a web browser application 238 for accessing, viewing, and interacting with web sites;
other applications 240, such as applications for word processing, calendaring, mapping, weather, stocks, time keeping, virtual digital assistant, presenting, number crunching (spreadsheets), drawing, instant messaging, e-mail, telephony, video conferencing, photo management, video management, a digital music player, a digital video player, 2D gaming, 3D (e.g., virtual reality) gaming, electronic book reader, and/or workout support; and
one or more data modules 242 for managing the storage of and/or access to data such as medical data, clinical data, patient data, and user data. In some embodiments, the one or more data modules 242 include:
   one or more medical databases 244 for storing medical data (e.g., regarding therapies, drugs, treatments, patients, cohorts and/or diseases); and
   one or more user databases 246 for storing user data such as user preferences, user settings, and other metadata.

In some embodiments, the agent modules 226 are configured to engage with a user in an integrated, conversational manner using natural language dialog, and/or invoke external services when appropriate to obtain information or perform various actions.

Referring to FIG. 2B, in some embodiments, the platform 100 provides an agent library 250 that includes a plurality of agent modules 226 (and/or the agent module(s) 316) and a system for managing and deploying these agent modules, such as through various blocks (e.g., agent builder blocks) realized in the form of one or more nodes 256.

In some embodiments, a respective agent module 226 (or agent module 316) is associated with a defined domain of information and/or a task-specific capability, which allows for retrieving a particular agent module based on information determined from a prompt provided by a user and/or based on a selection of the agent module by the user. In some embodiments, an agent module 226-1 is configured for a first specific task (e.g., generating a summary report of a patient's medical records), a second agent module 226-2 is configured for a second specific-task (e.g., generating a set of embeddings from summary data), a third agent module 226-3 is configured for a third specific-task (e.g., generating patient care guidelines based on a patient's health profile), a fourth agent module is configured for a fourth specific-task (e.g., identifying important dates for a patient based on summary data), a fifth agent module is configured for a fifth specific-task (e.g., identifying changes in a standard of care for a disease setting), a sixth agent module is configured for a sixth specific-task (e.g., evaluating unstructured data associated with a patient to identify a cohort of similar patients), and a seventh agent module is configured for a seventh specific-task (e.g., phenotyping a subject). In some embodiments, the agent library 250 includes N agent modules, where N is a positive integer. In some embodiments, the agent library 250 is stored at one or more client devices 102 and/or the server system 106 (e.g., a first portion of the agent library 250 may be stored at a first client device 102, a second portion of the agent library 250 may be stored at a second client device 102, and a third portion of the agent library 250 may be stored at the server system 106). In some embodiments, each agent module 226 includes a client-side portion and a server-side portion (e.g., a corresponding agent module 316 at the server system 106).

In some embodiments, each agent module 226 provides range of content and functionality that an end-user can engage with and/or configure for such engagement through one or more nodes 256 associated with the agent module 226, from a simple static response to sophisticated knowledge systems that facilitate automated conversations and data analysis leading to solutions and integrated transactions with external systems. Collectively, the one or more nodes 256 form some or all of a node architecture 254 associated with the agent module 226, which defines rules for traversing between nodes. In some embodiments, each respective agent 226 has a corresponding node architecture 254, which provides a one-to-one relationship between agent modules 226 and node architectures 254. In some embodiments, a respective agent module 226 supports the generation of additional agent modules 226 that utilize one or more models 252 and/or nodes 256 of a node architecture 254 of the respective agent module 226 or a different agent module 226. In some embodiments, a respective agent module 226 supports integration with other agent modules 226 in the agent library 250.

In some embodiments, each agent module 226 provides a defined scope for engaging in a workflow. Accordingly, in some embodiments, each agent module 226 is configured to assist end users to either resolve a question and/or problem or to fulfill a specific request for retrieving information, such as through a conversational communications framework. In some embodiments, a first subset of agent modules 226 are task specific and/or modality specific, whereas a second subset of the agent modules 226 are multi-modal and/or configured to perform multiple types of tasks. Some embodiments provide an ability to create, manage, and administer agent modules 226 to make them available for use in creating, editing, or deleting agent modules 226 via a user interface, e.g., by using a user-interface-based agent module builder or the like.

Some embodiments provide a user-interface-based agent module designer to assist in the creation and editing of agent modules 226 and/or a workflow associated with a variety of agent modules 226 (the workflow is also sometimes also referred to as an assembly or orchestration). In some embodiments, this workflow is manifested as a node architecture that includes a plurality of interconnected nodes. In some embodiments, the agent module designer includes the ability to define the name of an agent module 226, create an agent module 226, edit an agent module 226, delete individual nodes 256 associated with an agent module 226, expand and/or collapse node 256 branches, the ability to see and edit the conditional logic for a node 256, and the ability to see node traversals (e.g., when one or more nodes 256 connect to a different node 256).

In some embodiments, a node 256 of an agent module 226 reflects one or more decision points within an agent module 226, such as one or more predetermined decision points. In some embodiments, an agent module 226 evaluates data (e.g., a prompt provided by a user at a client device 102, an output from a different agent module 226, etc.), such as graphical data from a client device 102 by parsing and/or evaluating the incoming data for recognized keywords, phrases, ground truth labels, etc. For example, based on detection of recognized features, an agent module 226 may process information associated with the data received from the client device 102 in a particular direction within the plurality of interconnected nodes 256, such as from a node 256-1 associated with an agent module 226-1 to a node 256-2 associated with the agent module 226-1 and/or from the node 256-1 associated with the agent module 226-1 to a node 256-K associated with the agent module 226-1. Thus, in some embodiments, the use of one or more nodes 256 associated with a respective agent module 226 in a plurality of interconnected nodes 256 is similar to walking through a decision tree, where the different nodes 256 may be associated with different agent modules 226. Each agent module 226 may evaluate information based on associated conditional logic to progress information in the plurality of interconnected nodes 256. However, the present disclosure is not limited thereto. In some embodiments, each node in the plurality of interconnected nodes 256 comprises conditional logic that can evaluate data, retrieve data, generate data, or a combination thereof, e.g., based on an evaluation of information inputted to the respective node 256. In some embodiments, each node in the plurality of interconnected nodes 256 takes some action, such as generating a message and/or sending information to another node 256 in the same agent module 226 as the respective node, or a different node 256 of another agent module 226, or the like.

In some embodiments, a corresponding node architecture 254 associated with one or more respective agent modules 226 defines conditional logic 260, e.g., for performing a specific task (e.g., a specific clinical task). For example, each respective node 256 may include corresponding logic 260, which defines a workflow for handling one or more tasks assigned to the respective node 256. In some embodiments, the conditional logic of the node architecture 254 is executed in accordance with a first order of a first set of interconnected nodes 256 from a plurality of nodes 256 based on the corresponding logic 260 of each node 256 in the set of interconnected nodes 256. Accordingly, the logic 260 allows for granular configuration of each respective node 256 that when collectively coupled through interconnected nodes of the node architecture 254, define a conditional logic of the node architecture. For example, the logic 260 may include one or more logical operations or functions, such as AND, OR, XOR, and/or NOT operations (and/or any of the functions 280 shown in FIG. 2C). As an example, logic 260 for a node 256 may require the presence of a first condition but not a second condition or third condition.

In some embodiments, the plurality of nodes includes one or more data source nodes 256 associated with a specific task of obtaining data elements from a remote data source (e.g., an external database 108). In some embodiments, the corresponding logic 260 allows for connecting to a corresponding database, e.g., by using an access token associated with the corresponding agent module 226, communicating at least a portion of the obtained data to one or more nodes 256, and/or execute one or more queries to identify/analyze such data. In some embodiments, each node architecture 254 includes at least one input node, which forms an initial terminal node in an order of nodes 256. In some embodiments, the node architecture includes a plurality of paths to traverse from an input to an output node, such as paths of branching trees. In some embodiments, each respective node 256 represents a computational process, such as a function, an input, an output, or the like, that is realized when data is applied to the node 256. Moreover, since each node is interconnected, such by an edge, to at least one other node 256, the output from one node 256 may be supplied as input to a different node 256 in order to form chains and/or orders in the node architecture 254.

In some embodiments, the memory 218 includes one or more modules not shown in FIGS. 2A and 2B. For example, the memory 218 may include one or more agent tools (e.g., a communication tool) that are distinct from the agent modules 226. In some embodiments, the client device 102 includes one or more standalone agents (e.g., that execute and operate at the client device 102) and/or one or more dependent agents (e.g., that operate in conjunction with a component at a remote device, such as the server system 106). In some embodiments, one or more agents are generated/trained at the server system 106 and deployed at the client device 102.

Although FIGS. 2A and 2B illustrate the client device 102 in accordance with some embodiments, FIGS. 2A and 2B are intended more as a functional description of the various features that may be present in a client device than as a structural schematic of the embodiments described herein. In practice, and as recognized by those of ordinary skill in the art, items shown separately could be combined and some items could be separated.

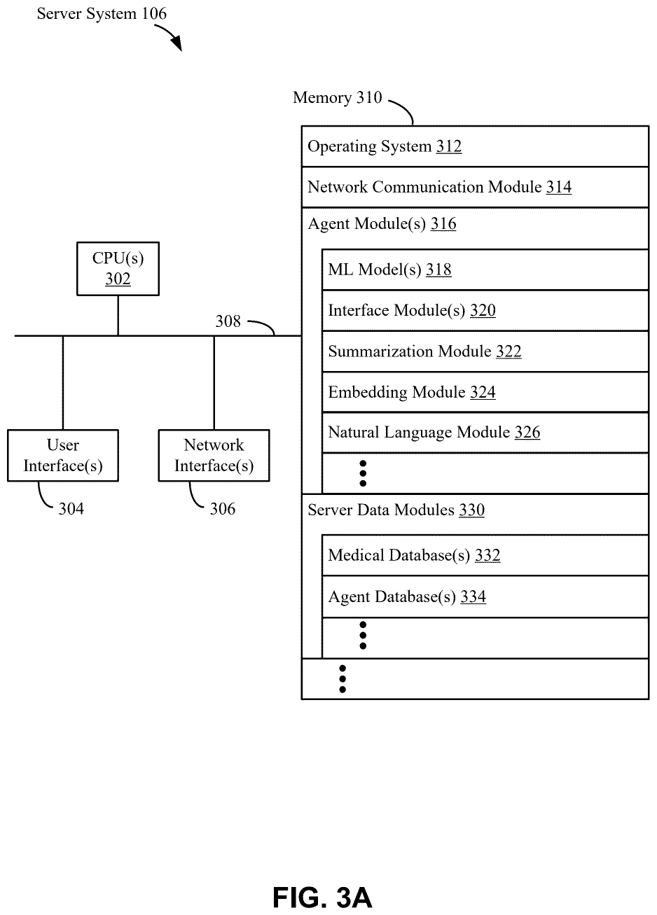

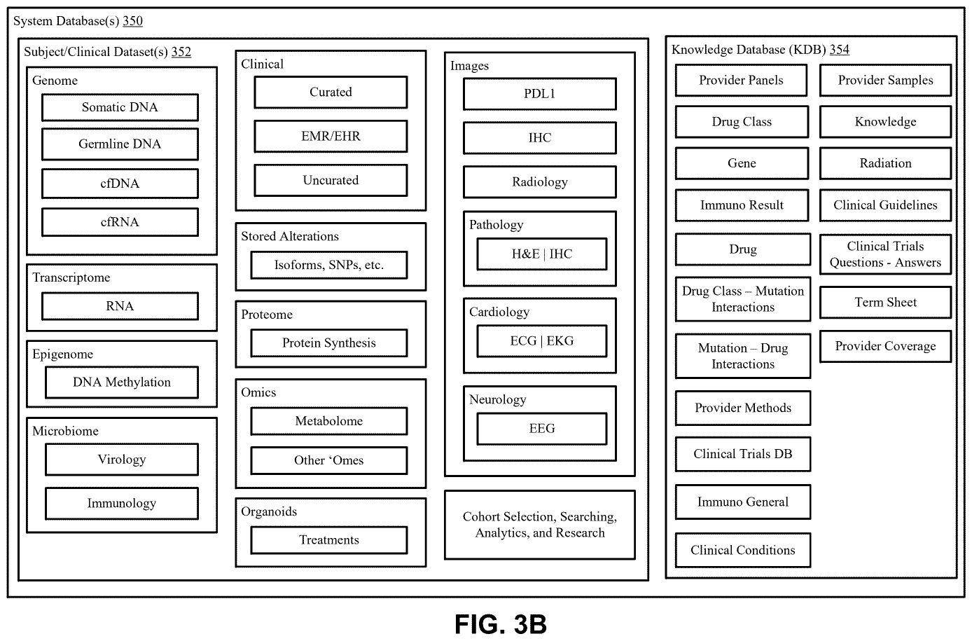

FIG. 3A is a block diagram illustrating a server system 106 in accordance with some embodiments. In accordance with some embodiments, the server system 106 includes one or more CPUs 302, one or more user interfaces 304, one or more network interfaces 306, memory 310, and one or more communication buses 308 for interconnecting these components. In some embodiments, the server system 106 includes other types of control circuitry and/or processors (e.g., in addition to, or alternatively to the CPUs 302). For example, the server system 106 may include one or more GPUs or DPUs for machine learning tasks.

The memory 310 includes high-speed random-access memory, such as DRAM, SRAM, DDR RAM, or other random access solid-state memory devices; and may include non-volatile memory, such as one or more magnetic disk storage devices, optical disk storage devices, flash memory devices, or other non-volatile solid-state storage devices. The memory 310 optionally includes one or more storage devices remotely located from one or more CPUs 302. The memory 310, or, alternatively, the non-volatile solid-state memory device(s) within the memory 310, includes a non-transitory computer-readable storage medium. In some embodiments, the memory 310, or the non-transitory computer-readable storage medium of the memory 310, stores the following programs, modules and data structures, or a subset or superset thereof:
an operating system 312 that includes procedures for handling various basic system services and for performing hardware-dependent tasks;
a network communication module 314 that is used for connecting the server system to other computing devices connected to one or more networks 104 via one or more network interfaces 306 (wired or wireless);
agent module(s) 316 that may engage with a user (e.g., a remote user) and invoke external services when appropriate to obtain information or perform various actions (e.g., in an integrated, conversational manner using natural language dialog). In some embodiments, the agent module(s) 316 work in conjunction with the agent module(s) at a client device 102. In some embodiments, the agent module(s) 316 include the following submodules (or sets of instructions), or a subset or superset thereof:
   one or more models 318 that engage with a user and/or perform specific tasks (e.g., in furtherance of a user request or query). In some embodiments, the model(s) 228 include one or more large language models, such as GPT-3, GPT-4, BioGPT, and PaLM-2, neural networks, transformer models, and/or other types of ML models; and
   one or more interface modules 320 that allow the agent module 316 to communicate with other agents, applications, components, and devices (e.g., via an API or structured query);
   a summarization module 322 that is configured to summarize one or more modalities of data, such as summarizing of a medical visit, annotating and/or labeling images, and/or otherwise summarizing data (e.g., in a human-readable format, such as a natural language summary);
   an embedding module 324 that is configured to generate embeddings (e.g., vectors) based on input data, such as raw input data and/or summarized input data. In some embodiments, the embedding module 324 is configured to generate modality-specific embeddings. In some embodiments, the embedding module 324 is configured to generate multi-modal embeddings (e.g., by aggregating or combining modality-specific embeddings); and
   a natural language module 326 that is configured to generate natural language (e.g., conversational) outputs. In some embodiments, the natural language module 326 is configured to convert one or more ML outputs into a natural language output. In some embodiments, the natural language module 326 is configured to generate embeddings from natural language inputs (e.g., received from a user via a digital assistant interface);
one or more server data modules 330 for managing the storage of and/or access to data (e.g., clinical and user data). In some embodiments, the one or more server data modules 330 include:
   one or more medical databases 332 for storing medical data (e.g., regarding therapies, drugs, treatments, patients, cohorts, imaging, and/or diseases); and
   one or more agent databases 334 for storing agent data such as settings, training, instructions, and other metadata.

In some embodiments, the server system 106 includes web or Hypertext Transfer Protocol (HTTP) servers, File Transfer Protocol (FTP) servers, as well as web pages and applications implemented using Common Gateway Interface (CGI) script, PHP Hyper-text Preprocessor (PHP), Active Server Pages (ASP), Hyper Text Markup Language (HTML), Extensible Markup Language (XML), Java, JavaScript, Asynchronous Javascript and XML (AJAX), XHP, Javelin, Wireless Universal Resource File (WURFL), and the like.

In some embodiments, the memory 310 includes one or more modules not shown in FIG. 3A. For example, the memory 310 may include one or more agent tools (e.g., a translation tool) that are distinct from the agent module(s) 316. In some embodiments, the server system 106 includes one or more standalone agents (e.g., that execute and operate at the server system 106) and/or one or more dependent agents (e.g., that operate in conjunction with a component at a remote device, such as a client device 102). In some embodiments, the memory 310 includes an agent library (e.g., the agent library 250).

Although FIG. 3A illustrates the server system 106 in accordance with some embodiments, FIG. 3A is intended more as a functional description of the various features that may be present in a server system than as a structural schematic of the embodiments described herein. In practice, and as recognized by those of ordinary skill in the art, items shown separately could be combined and some items could be separated. For example, some items shown separately in FIG. 3A could be implemented on single servers and single items could be implemented by one or more servers. In some embodiments, the clinical database(s) and/or the agent database(s) 334 are stored on devices that are accessed by the server system 106 (e.g., the external database(s) 108). The actual number of servers used to implement the server system 106, and how features are allocated among them, will vary from one implementation to another and, optionally, depends in part on an amount of data traffic that the server system manages during peak usage periods as well as during average usage periods.

Each of the above identified modules stored in the memory 218 and 310 corresponds to a set of instructions for performing a function described herein. The above identified modules or programs (e.g., sets of instructions) need not be implemented as separate software programs, procedures, or modules, and thus various subsets of these modules may be combined or otherwise re-arranged in various embodiments. In some embodiments, the memory 218 and 310 optionally store a subset or superset of the respective modules and data structures identified above. Furthermore, the memory 218 and 310 optionally store additional modules and data structures not described above.

As used herein, a transformer model (sometimes referred to as a transformer) is a neural network that learns context and thus meaning by tracking relationships in sequential data like the words in this sentence. Transformer models can apply attention, or self-attention, to detect how distant data elements in a series influence and depend on each other. Using embeddings (e.g., word embeddings), transformers can pre-process text as numerical representations through the encoder and understand the context of words and phrases with similar meanings as well as other relationships between words such as parts of speech. The models can then apply this knowledge of the language through the decoder to produce a unique output. Transformer models may be components of another model, such as a large language model (LLM).

An LLM is a large deep learning model that is pre-trained on large amounts of data, for example, in the size range of terabytes or even pentabytes. An LLM may have billions or trillions of parameters. LLMs typically consist of dozens or even hundreds of transformer blocks stacked on top of each other. In a classic LLM, each LLM includes an encoder block that takes a sequence and processes it into a set of context-rich embeddings, and a decoder block that takes the encoder's output and generates the output sequence. However, some LLMs include transformer blocks that only include an encoder and some LLMs include transformer blocks that only include a decoder. The transformer architecture makes use of self-attention, residual connections, and normalization. LLMs, which include stacks of transformer blocks, therefore make use of these features as well. Whereas a transformer model has in the order of millions of parameters, a large language model is characterized by having at least 1 billion parameters. As is apparent to one of skill in the art, these values exist in a continuous stream, e.g., there may be LLMs with 100 million parameters, 50 transformer blocks, or other numbers of parameters that allow for the robust performance expected of LLMs. As an example, a transformer model may have between 6 to 24 transformer blocks and an LLM may have 80 or more transformer blocks. As another example, a transformer model may be trained on domain-specific datasets that range in size between gigabytes and tens of gigabytes and an LLM may be trained on more diverse datasets that are measured in terabytes or pentabytes.

Embeddings are representations of values or objects (e.g., text, images, and/or audio) that are used by machine learning models. Thus, embeddings may represent features extracted from raw data. Embeddings may be (feature) vectors generated to capture meaningful data about each object. An embedding may be a word embedding that represents a word (or phrase) and is used in text analysis. The word embedding may be in the form of a real-valued vector that encodes the meaning of the word in such a way that the words that are closer in the vector space are expected to be similar in meaning. In the case where words and phrases are one-hot encoded, an embedding is typically dimension reduced relative to the model input. For example, consider the case where a model has a vocabulary size of 50,000 words and/or phrases. Words and phrases in model input are one-hot encoded using this vocabulary and thus the input has a dimension of 50,000. In some models in accordance with the present disclosure, such high-dimensional input is dimension reduced relative to the original one-hot input. For instance, in one particular example the embedding maps the 50,000 word/phrase vocabulary to 768 dimensions. However, there is no absolute requirement that an embedding be dimension reduced relative to the input. For instance, in some embodiments the embedding captures input context, resulting in embeddings that are not dimension reduced relative to the input.

FIG. 3B is a block diagram illustrating one or more system databases 350 in accordance with some embodiments. In some embodiments, at least a portion of the system database(s) 350 is stored at a client device 102 (e.g., as the medical database(s) 244), the server system 106 (e.g., as the medical database(s) 332), and/or the external database(s) 108, which advantageously allows for an edge at and/or near the client device 102, such as via the communication network. However, the present disclosure is not limited thereto. In some embodiments, a single database stores all of the information shown in FIG. 4. In some embodiments, the information is stored in a set of two or more databases.

In some embodiments, the system database(s) 350 include subject and clinical datasets 352 and/or a non-subject specific knowledge database (KDB) 354. In some embodiments, the data stored in the system database(s) 350 includes a plurality of categories of data or data features, the categories of data or data features encapsulating the different data modalities such as a structured text modality, an unstructured text modality, a tabular data modality, a data visualizations modality, an image modality, an audio modality, a video modality, a biological sequence modality, a natural language modality, and a source code modality. In some embodiments, the data stored in the system database(s) 350 includes raw data (e.g., unstructured data corresponding to entire documents in original formatting). In some embodiments, the data or features stored in the system database(s) 350 includes formatted (e.g., structured data) and/or summarized data (e.g., summaries generated by one or more modality-specific summary agents). In some embodiments, the system database(s) 350 include data or features stored in an embedding format (e.g., a numerical vector format).

In some embodiments, the datasets 352 include, among other data, genome, transcriptome, epigenome, microbiome, clinical, stored alterations proteome, additional-omics, organoids, imaging and cohort and propensity data sets. For example, the cohort selection, searching, analytics, and research datasets may include data about patients and conditions, such as tumors of unknown origin (TUO) predictors, metastasis predictors, and survival analytics. As an example, the imaging datasets may include radiology imaging data, immunohistochemistry imaging data, positron emission tomography (PET) data, pathology imaging data, cardiology imaging data, neurology imaging data, and/or single-photon emission computed tomography (SPECT) imaging data. The pathology imaging data may include hematoxylin and eosin (H&E) and/or Immunohistochemistry (IHC) data. The cardiology imaging data may include electrocardiogram (ECG or EKG) data. The neurology imaging data may include electroencephalogram (EEG) data. The imaging datasets may include data regarding nodule identifiers, tracking, and/or longitudinal analytics. The imaging datasets may also include data regarding whole slide staining using hematoxylin and eosin (H&E) or immunohistochemistry (IHC) stains and/or pathology reports. The clinical data may include curated, uncurated, electronic medical record (EMR), and/or electronic health record (EHR) data. The uncurated data may include raw images of documents which can be OCRed and then fed to a model for structuring/summarizing. In some embodiments, the same model performs the OCR and structuring/summarizing, such as a LLM, transformer, neural network, or machine learning model.

In some embodiments, the clinical data includes diagnostics, imaging, biopsy information, and other disease-and condition-related data. For example, for endocrinology diagnostics, the primary test used may be a blood test to measure hormone levels in the body, which can identify various endocrine disorders by checking for imbalances in hormones such as thyroid stimulating hormone (TSH), luteinizing hormone (LH), follicle stimulating hormone (FSH), testosterone, and others depending on the suspected condition. Additional tests such as ultrasounds, CT scans, or biopsies may be performed depending on the situation, e.g., to locate abnormalities in endocrine glands like the thyroid or adrenal glands. Blood tests for endocrinology diagnostics can be used to measure various hormones in the blood, allowing diagnosis of conditions like hypothyroidism, hyperthyroidism, diabetes, and adrenal insufficiency. Imaging tests such as ultrasounds, CT scans, or MRIs can be used to visualize the endocrine glands and identify abnormalities like nodules or tumors. A fine needle aspiration (FNA) biopsy may be performed to collect a tissue sample from a suspicious area in the thyroid gland for further analysis. Thyroid function tests may be used to measure TSH, T4, and T3 levels to assess thyroid function. Cortisol level tests may be used to check for adrenal gland issues. Glucose tolerance tests may be used to diagnose diabetes by monitoring blood sugar levels, e.g., after consuming a sugary drink. Prolactin tests may be used to check for prolactin levels associated with pituitary gland disorders. Calcium and parathyroid hormone (PTH) levels may be determined to assess parathyroid gland function. For each endocrinology-related test, the data relating to the test (e.g., diagnostics, imaging, and metadata (such as timing, location, etc.)) may be stored in the clinical data, and associated with a particular subject.

As another example, for diabetes diagnostics, a doctor may use a blood test, such as the Hemoglobin A1c (A1C) test, which measures average blood sugar level over the course of two to three months. The A1C test provides a snapshot of a subject's average blood sugar over a period of time and does not require fasting. Other tests may be used, such as a fasting blood sugar test, an oral glucose tolerance test (OGTT), or a urine test, depending on the situation. The fasting blood sugar test measures a subject's blood sugar level after fasting for at least 8 hours. The OGTT involves the subject drinking a sugary liquid and then having their blood sugar levels checked at specific intervals. While not as accurate as blood tests, a urine test may be used in some situations to check for ketones, a sign of uncontrolled diabetes, particularly in type 1 diabetes. For each diabetes-related test, the data relating to the test may be stored in the clinical data, and associated with a particular subject.

As another example, to diagnose and/or assess depression a variety of tests and tools can be used, including questionnaires, physical exams, lab tests, and brain scans. For example, the Patient Health Questionnaire (PHQ-9) is a questionnaire that can help diagnose depression and assess its severity. The PHQ-2 is an initial screening tool for depression that can be used in all age groups. Other questionnaires include the Social Problem-Solving Inventory-Revised (SPSI-RTM), which is a self-report measure of social problem-solving strengths and weaknesses. The Edinburgh Postnatal Depression Scale (EPDS) is a 10-question scale that can be used to screen for depression in women who have recently given birth. In some situations, a doctor or other mental health professional may perform a physical exam and ask questions about a subject's health to diagnose/assess depression. A mental health professional may also use the criteria for depression listed in the Diagnostic and Statistical Manual of Mental Disorders (DSM-5). In some situations, lab tests are used to rule out other medical conditions that could be presenting as depression. These tests may include a complete blood count (CBC), thyroid-stimulating hormone (TSH), vitamin B-12, and the like. Additionally, a PET scan of the brain can compare brain activity during periods of depression with normal brain activity. A CT scan or MRI of the brain may be considered if organic brain syndrome or hypopituitarism is in the differential diagnosis. For each depression-related test, the data relating to the test may be stored in the clinical data, and associated with a particular subject.

As another example, there are different types of diagnostic tests that can be used to diagnose cardiovascular disease, including Electrocardiograms (ECG or EKG), longitudinal Holter monitoring, stress tests, cardiac MRIs, cardiac positron emission tomography (PET) scans, invasive coronary angiographies, echocardiograms, blood tests, x-rays, cholesterol tests, c-reactive protein tests, trimethylamine N-oxide tests, serum creatinine, and plasma ceramides tests. A doctor may use a combination of tests to diagnose a heart problem. For example, a doctor might use an echocardiogram, cardiac MRI, or a nuclear heart scan to take images of the heart during or after a stress test. For each test, the data relating to the test (including any comparisons, cross references, and conclusions based on multiple tests) may be stored in the clinical data, and associated with a particular subject.

In some embodiments, the KDB 354 includes separate sub-databases related to specific information types including, as shown, provider panels (e.g., information related to genetic panels supported by the service provider that operates the system), drug classes (e.g., drug class specific information (e.g., do drugs of a specific class work on pancreatic cancer, what drugs are considered to be included in a specific drug class, etc.)), specific genes, immuno results (e.g., information related to treatments based on specific immuno biomarker results), specific drugs, drug class-mutation interactions, mutation-drug interactions, provider methods (e.g., questions about processes performed by the service provider), clinical trials, immuno general, clinical conditions such as clinical diseases, term sheets (e.g., definitions of industry specific terms), provider coverage (e.g., information about provider tests and results), provider samples (e.g., information about types of samples that can be processed by the provider), knowledge (e.g., scripted questions and answers on various frequently asked questions that do not fall into other sub-databases), radiation (e.g., information related to suitable radiation treatments given specific cancer states), clinical guidelines (e.g., national guidelines related to classification of cancer states, accepted treatments, etc.) and clinical trials questions-answers (e.g., information related to locations and administrators of clinical trials. Organizing the KDB 354 into sub-databases may make it easier to manage those databases as information therein evolves over time and also enables addition of new sub-databases related to other defined information types. In some embodiments, the clinical datasets 352 and/or the KDB 354 is arranged in a different manner than is shown in FIG. 4 (e.g., with different sub-databases and/or with a different organizational scheme).

In some embodiments, the data stored in the subject and clinical datasets 352 and/or the KDB 354 is includes raw data, annotated data, and/or summarized data. In some embodiments, the raw data is input into one or more models to generate the annotated and/or summarized data. For example, a model may receive raw data, such as sequencing results, documents, and/or images, and extract/predict status information and/or summaries. In some embodiments, one or more models (e.g., one or more agents) are used to partition, annotate, summarize, and/or structure the data received from external sources (e.g., external databases and/or third parties). In some embodiments, the data stored in the subject and clinical datasets 352 and/or the KDB 354 is classified, grouped, cross-referenced, and/or otherwise related to other data using one or more models (and/or one or more agents). For example, a cohort may be identified based on EMR/EHR information from multiple subjects/patients. In some embodiments, an intake agent is used on data that is received to perform one or more of the actions described above. In some embodiments, different intake agents (e.g., data processing/pre-processing agents) are used for different modalities of data.

Advantageously, by utilizing multiple datasets associated with different domains of subject matter and/or applying a classification system to the datasets, the knowledge database provides a storage system for data, such as medical records and clinical documentation that one or more agent modules 226 can retrieve based on a task-specific requirement associated with a respective domain or classification. Moreover, in some embodiments, the knowledge database 354 allows for storing such data with deidentifying controls in order to allow for training on and/or analysis of the stored data without risk of leaking confidential and/or privileged information.

Considering the extensive volume of text contained within a real-world data (RWD) warehouse of EHRs, it becomes impractical to process the entirety of a patient's clinical notes within the context window of a model (e.g., an LLM). In some embodiments, this challenge is addressed by implementing a retrieval-augmented generative (RAG) approach to identify relevant portions of EHR text, e.g., relevant portions of unstructured clinical notes. A RAG approach proves to be more efficient and effective than providing the model with larger context windows. In some embodiments, RAG is a two-step process that involves retrieving relevant documents from a corpus (e.g., a large corpus with thousands or millions of documents) and then feeding the retrieved documents into a model to generate an analysis and response.

In some embodiments, one or more of the agent modules of the agent library 250 use a retrieval-augmented generative (RAG) to perform operations described herein (e.g., requests to process zero-shot information). For example, the computing system may apply the RAG process to entire patient records, which allows for applying the entire patient records to a model 228 with excess computational burdens, as opposed to focusing solely on a specific type of clinical note. In some embodiments, the RAG process is used to analyze clinical mentions throughout a patient's entire record without the need for predefined sections of interest. However, the present disclosure is not limited thereto. In some embodiments, the RAG process utilizes one or more vector embeddings, such as a plurality of predetermined vector embeddings in which each predetermined vector embedding is associated with a corresponding text string, or snippet. Advantageously, this RAG approach can be more efficient and effective than providing a model (e.g., an LLM) with larger context windows.

In some embodiments, one or more of the agent modules use additional techniques to address an issue that RAG implementations can fail to obtain all of the needed information to fully answer a question (e.g., a user query). In such situations, another request (e.g., a new user query, and/or a modified version of the user query) can be automatically generated to cause more information to be obtained. An example technique includes applying a user query for information from a source dataset to a first RAG agent (e.g., one or more agent module(s) 226) to determine if there is enough information to generate an output based on the user query. The RAG agent can determine that there is enough information, that there is not enough information, or that the determination is not clear. In some embodiments, if the determination whether there is enough information is not clear, the computing system provides a query to a different task-specific orchestration (e.g., corresponding to a different agent module 226). That is, in some embodiments, the system determines that the RAG agent may not be the optimal instrumentation for resolving the user query.

In some embodiments, operations of one or more task-specific orchestrations of the system are adjusted to reduce/prevent negative consequences of retrieval-augmented generation. For example, for some inclusion/exclusion criteria for trial matches or care gap discovery, the queries have a relationship and can include a temporal question (e.g., “Is this medication administration currently administered as the first line of therapy?”). As another example, with a standard RAG retrieval approach, only documents relevant to medications may be retrieved. But the task-specific orchestration (e.g., the RAG agent) may not know if the medications were administered as part of the first or second line of therapy without the full context of the patient. In such situations, using a large context where most of the patient notes can be applied can provide the task-specific orchestration better context and more comprehensive information about the temporal relationship between events. Alternatively (e.g., to address the resource constraints of increasing the context window applied to the RAG agent), a different model (e.g., a full patient record LLM with a one-million-character context window) or agent can be used to resolve the user query in addition or alternatively to the RAG agent. For example, increasing the context window and/or performing additional operations alternative to directing a request to the RAG agent (e.g., to extract information) can increase performance of generating the output based on the user query for information. As another example, a particular subset of data may be used for verification of the data/results. For example, insurance claims data may be used to verify which medications are administered during particular times (e.g., corresponding to particular lines of treatment). In this way, insurance claims data may be used to identify transitions between different lines of treatment.

FIG. 4 illustrates an example system architecture for deploying agents (e.g., agent modules 226 and/or 316) in accordance with some embodiments. The architecture 400 shown in FIG. 4 includes an agent builder component in a control plane of a client device 102. The control plane may function as a supervisor of data, coordinating communication between different components and collecting data from a data plane (e.g., a working environment presented on a display of the client device 102). In some embodiments, the control plane resides above the data plane (e.g., above the working environments) and enforces rules for the data plane, which allows for partitioning the data plane to prevent unauthorized or unauthenticated control of the data plane from unsecure client devices, such as those unassociated with a portion of the data plane. However, the present disclosure is not limited thereto. In some embodiments, the agent builder hosts a user interface for configuring agent modules, such as by configuring the corresponding node architecture 254 associated with the agent module 226. In some embodiments, the agent builder component is communicatively coupled to an agent library (e.g., the agent library 250) in the control plane that stores a plurality of agent modules, such as the agent module 316, and to an agent host (e.g., via a configuration publication/subscribe (pubsub) component) in a working environment. The agent module in the working environment may be communicatively coupled to an agent library in the working environment, a document index (e.g., one or more data sources, such as knowledge database 354 and/or external databases 108), and a large language model (e.g., a model 318). In some embodiments, the agent library 250 includes a user interface and API for interacting with deployed agents.

In some embodiments, the agent builder includes a frontend and a backend. In some embodiments, the agent builder frontend includes an access component (e.g., an administrative console that may be a home user interface that a user is presented with upon providing access credentials to the application), an agent list (e.g., an agent library that may include a plurality of orchestrations to which the user has access, e.g., based on the access credentials provided to the application), an agent builder component (e.g., including a first representation of a node architecture 254 (e.g., a form-builder representation) and a second representation of the node architecture 254 (e.g., a workflow representation)), and/or a data source management component. In some embodiments, the agent builder backend includes a database layer, an API service, and/or a configuration publisher component. In some embodiments, the frontend and the backend of the agent builder are executed on separate electronic devices.

In some embodiments, the agent host includes a frontend and a backend. In some embodiments, the agent host frontend includes an access component, an agent list, an interaction console, and/or a document console. In some embodiments, the agent host backend includes a websocket for interactive user, a database layer, an API access to deployed agents, tools and/or custom chain implementation, a document loader, and/or a configuration subscription component. In some embodiments, the frontend and the backend of the agent host are executed on separate electronic devices.

In some embodiments, the agent builder component is configured to generate, deploy, and/or update one or more agent modules and/or a corresponding node architecture to one or more working environments (e.g., one or more workload planes). In some embodiments, each agent module is associated with an agent type. In some embodiments, the agent type includes a type of model and/or conditional logic, such as an implementation configuration. For example, an agent module may include a language model associated with a first node and a corresponding type-specific logic that further associates the agent module, through the first node, with a particular domain, such as a first configuration implementation for applying the prompt to the model if the prompt is associated with a first modality and a second configuration implementation if the prompt is associated with a second modality different from the first modality. In some embodiments, the logic is specified in a corresponding agent module configuration file, which advantageously allows for configuring the logic after applying various prompts to the agent module and/or using multiple client devices (e.g., end users) to configure the logic. However, the present disclosure is not limited thereto.

In some embodiments, the available agent module types include a transform agent (e.g., performing functions such as data transformations, regular expressions, and string templating), an authorization agent, a language model agent (e.g., applying inputs to a large language model), a data collection agent (e.g., RAG modules), a super-agent (e.g., aware of other agent types and their capabilities and configured to instantiate and/or delegate to the appropriate agent modules), a sequential agent (e.g., including multiple models and/or tools coupled together in a sequential fashion), a tool-using agent, a coding agent (e.g., configured to generate code in particular programming languages), and/or a categorization agent (e.g., configured to determine an intent, domain, or other categorization for user inputs). In some embodiments, the transform agent comprises one or more ML models (e.g., stored as transforms accessible by the platform). In some embodiments, the one or more ML models are stored (e.g., as disk images) for subsequent initialization/instantiation. In some embodiments, the language model agent provides and/or stores context information such as conversation history, user preferences, subject details, and the like. In some embodiments, the data collection agent is couplable to external data sources (e.g., the external service(s) 110 and/or the external database(s) 108) and configured to request and/or retrieve data from the external data sources. In some embodiments, a sequential agent includes a recursive module (e.g., repeating and/or refining outputs until predetermined criteria are met). In some embodiments, a super agent is configured to compare available agent types and recommend a particular agent type for a particular situation/purpose. In some embodiments, a coding agent is configured to generate code for new agent modules based on inputs (e.g., natural language inputs) from a user. In some embodiments, a categorization agent is a component of a routing agent. For example, the categorization agent determines an intent/domain for an input and the routing agent routes the input to a downstream component in accordance with the determined intent/domain. In some embodiments, a sequential agent is a component of a routing agent. For example, the routing agent coordinates operation (e.g., data transmission and timing) of multiple components and/or modules. In some embodiments, each agent module is generated/provided with guardrails (e.g., enforcing privacy, security, data typing, etc.). In some embodiments, an agent module is configured to recognize whether data is protected health information (PHI) and take appropriate action. For example, an agent module may disable information sharing options when providing PHI.

In some embodiments, different agent types are associated with (e.g., trained on, instructed on, and/or coupled to) different domains (e.g., different subjects, types of data, modalities of data, and/or classes of data) in a plurality of domains. For instance, in some embodiments, the plurality of domains forms an input space, which defines a universe of data associated with a variety of subject matters. In some embodiments, the input space defines an N-dimensional space of data obtained from a plurality of data sources, in which N is a positive integer, such as two, three, four, ten, etc. In some embodiments, each respective domain in the plurality of domains defines a partition classification or subset of data, such as one or more specific data sets of system databases 350 of FIG. 3B. In some embodiments, different agent types are associated with (e.g., trained on, instructed on, and/or coupled to) different data modalities in a plurality of data modalities. However, the present disclosure is not limited thereto.

As a non-limiting example, consider a first input space associated with a plurality of medical records, in which each medical record in the plurality of medical records includes a plurality of text data and a plurality of graphical data associated with a corresponding patient. Accordingly, a plurality of domains collectively defined by information obtained from the plurality of medical records allows for classify the information and training a corresponding agent module on the information classified domain, such as a first domain associated with a statin drug class of FIG. 3B, and a second domain associated with a glucagon-like peptide (GPL) agonist drug class.

As a non-limiting example, a first agent module may be associated with a first domain for generating a summary of a patient's textual medical record, a second agent module may be associated a second domain for generating annotations and/or labels for graphical data (e.g., image data) in the patient's medical record, a third agent module may be associated with a third domain for generating annotations and/or labels for biological sequence data in the patient's medical record, and a fourth agent module may be associated with a fourth domain for generating inferences to user queries using the data generated by the other three agent modules. In another example, a first agent module may be associated with a first domain for generating a summary of a patient's textual medical record, a second agent module may be associated with a first domain for guiding a subject, such as a patient or a medical practitioner associated with the patient, through a care plan, a third agent may be associated with a third domain for creating patient care guidelines based on a patient's health profile, a fourth agent module may be associated with a fourth domain for identifying patients requiring follow-up at a hospital, a fifth agent module may be associated with a fifth domain for identifying changes in a standard of care for a disease setting, and/or a sixth agent module may be associated with a sixth domain for evaluating data associated with a patient to identify a cohort of similar patients.

One example agent type is a database-interfacing agent module associated with one or more data source nodes 256. An example database-interfacing agent may be an adverse effects agent that has access to an FDA label database and is configured to interpret adverse effect information from the database. The configuration of the database-interfacing agent module may include a custom prompt for the model(s) of the agent module and one or more data sources that the agent database-interfacing module may access and/or use.

Another example agent type is a custom-chain agent module (e.g., a super-agent module) that takes an input prompt, analyzes the prompt (e.g., parsing the prompt into one or commands and/or a plurality of tokens), and transmits information from the parsed prompt (e.g., commands and/or tokens) to a model or other component, such as a node 256 of the custom-chain agent module or a different node 256 of a different agent module). For example, the custom-chain agent module may obtain data from different databases (e.g., external databases 108, knowledge database 354, etc.), in which the data is obtained in a variety of different formats, modalities and/or structures, such as unstructured text, structured text, tables, charts, graphical data, and/or biological data. In some embodiments, the agent module reformats, summarizes, and/or restructures the data obtained from the databases for application to a model of the custom-chain agent module and/or to a different agent module. In some embodiments, the custom-chain agent module evaluates and/or obtains a set of parameters for inputting data to the model(s) and/or agent module(s) and translates the data obtained from the databases based on the set of parameters. In some embodiments, the obtained data is restructured into a homogenous dataset (e.g., different hospitals may use different codes for the same procedure, such is homogenized by the agent module into a uniform coding). The configuration of the custom-chain agent module may include a sequence of nodes 256 associated with the custom-chain agent module and/or other nodes associated with other agent modules to be used by the custom-chain agent module and/or definitions of corresponding chain objects.

As illustrated in the above examples, an agent module may be considered a configuration of a particular agent type for a particular task through a plurality of interconnected nodes 256 that form a node architecture 254 of the agent module (e.g., represented as a database object). An agent module may be configured for dissecting complex evaluations and logics into a reasoning path through the plurality of interconnected nodes 256, which makes arriving at an accurate and precise response computationally less burdensome. In some embodiments, the agent modules are accessible via an interaction console and/or an application programming interface (API). In some embodiments, one or more parts of the agent configuration are stored in a separate versioning table (e.g., linked by agent ID). In this way, an agent configuration may be edited without affecting a deployed agent version.

In an example scenario, a user configures an agent in the console and then deploys it to one or more environments (e.g., workload planes and/or control planes). For this scenario, the agent configuration is stored in the control plane (e.g., as shown in FIG. 4). As shown in FIG. 4, the agents themselves execute in the appropriate working environments, and working environments do not have access to the control plane. The agent builder in the control plane may be configured to push configurations into the various environments (e.g., via the config pubsub component shown in FIG. 4). In some embodiments, when an agent configuration is changed or an agent version is deployed, the agent builder informs the agent host in each environment so that the updated agent can be deployed. This may be via a pubsub message to the agent-config topic or via a simple HTTP request.

The architecture 400 allows for flexibility in supporting a variety of deployment strategies for each respective agent module. For example, some end-users, e.g., those using agent modules interactively and without engineering support, expect to operate their agent modules entirely within a production working environment. In some embodiments, the administrator, such as a creator, of an agent module is able to choose a deployment style suitable for their application, such as by restricting the agent module to one or more domains, one or more databases 108, one or more services 110, or a combination thereof. For example, a first user may wish to employ a user interface that includes one or more user interface elements described with respect to the application by directly embedding the components within a web page, and a second user may wish to interact with an API that is configured to receive user requests and provide responses in the form of data structures, which the second user may integrate into different user interface elements not associated with the application.

In some embodiments, users of an agent builder user interface in the control plane are provided with a production access token that can also make requests to the production agent host. In some embodiments, an integrated user interface is presented to a user that shows both the agent builder having a plurality of input features visualized through a representation and the interaction console without concerning the users with the differences between the control plane and the working environments. For example, for users who want to test out agent modules in a lower environment, a link may be provided to open that agent module in a new tab or frame of an application. In some embodiments, a request to authenticate is presented and an access token is obtained by the agent module for that environment. In some embodiments, the user interface includes an indication of which environment is currently active.

In some embodiments, a data module 410 (e.g., document index) as shown in FIG. 4 includes one or more of: a static corpus, a dynamic corpus, an embedding model (e.g., a model 228), a chunking strategy, a storage back-end, a data classifier (e.g., public, internal, or secret), and/or a visibility setting (e.g., private, public, or restricted by role). In some embodiments, the data module maintains an index of data that may be ephemeral or permanent. In some embodiments, data elements associated with data files (e.g., documents) are evaluated via a chunking process, embeddings are generated for the chunks generated from the chunking process, and the embeddings are inserted into a database. In some embodiments, the data module 410 includes a set of retrieval parameters (e.g., for a number of documents to retrieve and/or a similarity measure). In some embodiments, the data module 410 corresponds to a set of databases (e.g., medical databases), such as the database(s) 350 in FIG. 3B. In some embodiments, a parameter associated with a node 256 of a respective agent module and/or model includes selecting one or more document indices to retrieve from via the data module 410. In some embodiments, embeddings are created and siloed for future use. In some embodiments, each embedding is associated with one or more access control lists (ACLs).

Tools are a mechanism by which agent modules can integrate with other components and with the outside world. In some embodiments, tools are made available to the agent modules as agent builder blocks. Some tools may be general-purpose, and others may be custom for a particular integration. Different agent module types may have different access to tools: for example, a tools agent may be configured with a set of available tools, and the model may be configured to choose when and how to use them, rather than follow a fixed sequence of steps. In some embodiments, an agent configuration defines when and how tools are invoked. As an example, a tool may be configured with a fixed base URL so that the agent cannot make authentication requests to some other service. In some embodiments, a tool is configured to use an end-user's access token to authenticate, rather than granting an access role to the agent's machine user. In some embodiments, a tool is restricted to certain endpoints and/or methods (e.g., only GET requests) so that the tool is restricted from performing admin tasks on behalf of a user who lacks admin privileges (e.g., write permissions).

In some embodiments, a tool has parameters that are specified when configuring the agent modules and/or parameters that can be specified at invocation time by the agent module itself. An example tool is an authentication request tool configured to fetch an internal URL using a user's access token. The authentication request tool may include the following parameters: name, description, base URL, and/or input parameters (e.g., specifiable by the agent). For example, an example authentication request tool may have an order identifier as an input parameter. Another example tool is an external request tool that fetches an external URL. The parameters for the external request tool may include: name, description, base URL, and/or input parameters. Another example tool is an email tool that sends an email. The parameters for the email tool may include destination, subject, and/or body.

Other example agent modules include (i) an agent module configured to send emails summarizing which customers are facing issues with orders and/or identifying retraining opportunities, (ii) an agent module configured to generate data tables, JSON schema, and other data translations, (iii) an agent module configured to find orders within a group of clients that have particular flags and/or provide a summary by client, flag, etc. (e.g., with timestamp for order creation timing), (iv) an agent module for identifying behavioral changes in ordering habits and adjust orders accordingly (e.g., increase delays and/or cancel orders) and sending notifications, (v) an agent module for generating inclusion/exclusion criteria from a protocol document, generating structured queries (e.g., SQL queries) from a structured list, and/or generate specifications (e.g., YAML specifications) from structured lists of inclusion/exclusion criteria, and (vi) an agent module for answering questions about particular trials based on information in the protocol and/or other trial materials or documentation. As another example, a set of one or more agent modules may be configured to identify and/or evaluate adverse effects. The example agent module(s) receive a user query regarding adverse effects associated with a particular drug. In this example, the set of agent modules may parse the query in order to identify the drug name from the query and apply the drug name to one or more nodes in order to obtain a set of adverse effects associated with the drug. In this example, the set of agent modules may provide a response with a description of the set of adverse effects.

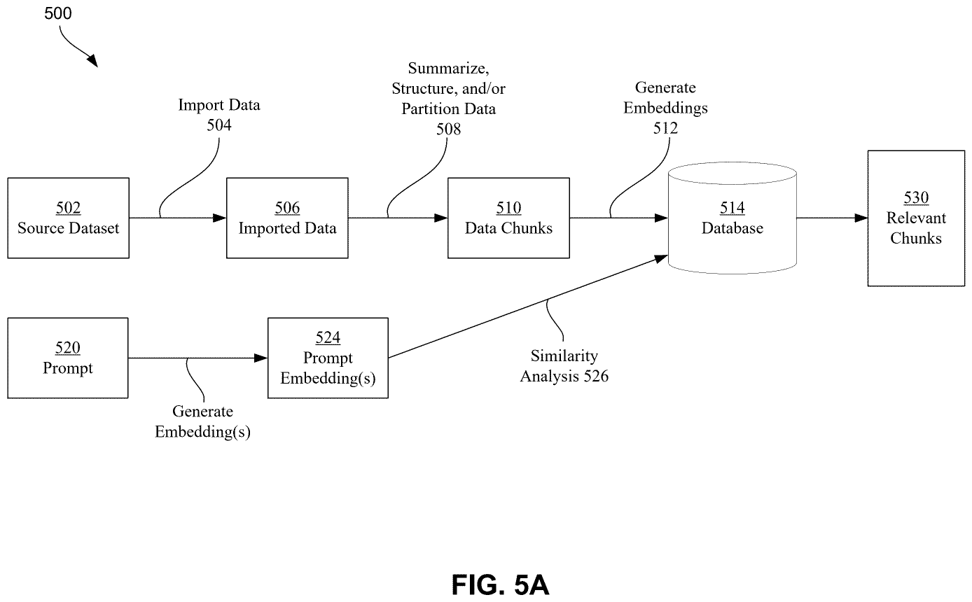

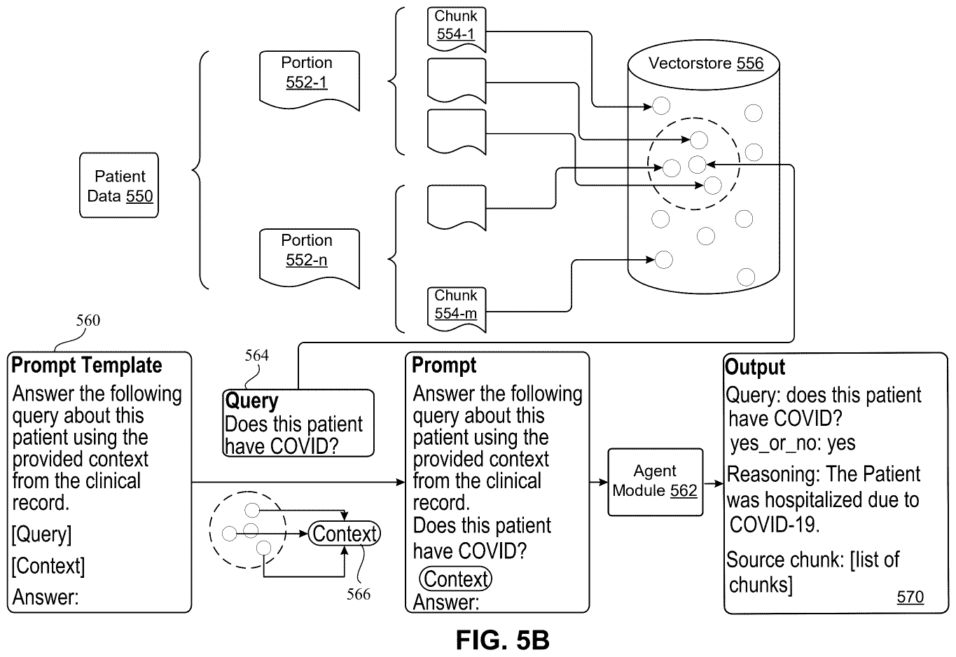

FIG. 5A illustrates an example process 500 for data vectorization and query processing in accordance with some embodiments. First, a source dataset 502 is imported (504) as imported data 506. In some embodiments, the source dataset 502 includes one or more documents (e.g., one or more PDF documents), one or more images, and/or other structured or unstructured data (e.g., data tables or records). In some embodiments, the source dataset 502 is obtained from one or more databases (e.g., the external database(s) 108). In some embodiments, the source dataset 502 is identified by a user for importation into the system (e.g., the platform 100). In some embodiments, the source dataset 502 includes medical, clinical, molecular, and/or patient data.

In accordance with some embodiments, the imported data 506 is de-identified (e.g., any personally identifiable information (PII) is removed). The imported data 506 is converted (508) into data chunks 510. In some embodiments, the conversion includes summarizing the imported data 506 (e.g., using one or more machine-learning models). In some embodiments, the conversion includes converting unstructured data into structured data (e.g., using one or more machine-learning models). In some embodiments, the conversion includes partitioning the data (also sometimes called chunking or snippetizing). For example, the imported data may be converted to structured data then summarized and then the summary data may be partitioned to generate the data chunks 510. In some embodiments, the imported data 506 is summarized, e.g., with or without being converted to structured data. In some embodiments, the imported data includes visual data that is annotated and/or characterized during the conversion process. A set of (one or more) embeddings are generated (512) from the data chunks 510 and stored in a database 512 (e.g., a vector database). In some embodiments, the embeddings are used to train (e.g., fine tune) a machine-learning model (e.g., a model that is a component of a task-specific orchestration).

FIG. 5A also shows a prompt 520 being received (e.g., via the platform 100). For example, the prompt may be a question about the source dataset 502. The prompt 520 is converted (520) to a set of (one or more) prompt embeddings 524. A similarity analysis 526 (e.g., a cosine similarity analysis) is performed between the prompt embedding(s) 524 and the embeddings in the database 514 (e.g., the embeddings from the data chunks 510). In this way, one or more relevant chunk(s) 530 are identified and may be returned to the user. In some embodiments, the relevant chunk(s) 530 are analyzed and/or summarized and the results of the analysis/summary are provided to the user. In some embodiments, the response to the user includes a short answer, a long answer, and/or information from the relevant data chunks 510.

As an example, a query vector may be generated and used to identify a similar vector in a vector database (e.g., the database 514). The similar vector from the query vector database (and/or the query vector) may be used to identify a second similar vector in a second vector database. The query vector and the second similar vector (and optionally the first similar vector) may be provided to a language model via a prompt. The language model outputs an answer to the query, which is, optionally reformatted, and transmitted to the user. In a specific example, the query is “what is the reason for Linda Watson's order cancelation” and the language model outputs a status reason as the answer.

In some embodiments, an agent module is configured to perform intent matching and/or parameter extraction on the user queries and requests. In some embodiments, the intent is assumed (e.g., the agent module is configured for a specific task). In some embodiments, the agent module extracts domain-specific parameters. For an example query “show patients with MSI high, TMB less than 20, which have been diagnosed with central neurocytoma in the past four months” the extracted parameters may be [“mis”: “high”, “tmb”: “{“It”“20”}, “diagnosis”: “central neurocytoma”, “date_range”: {. . . }].

In some embodiments, an agent module is configured to automatically populate a structured query (e.g., an SQL query) using a user query and transmit the structured query to a structured database. For example, the agent module may obtain a particular schema, obtain inclusion and exclusion criteria, and generate a structured query for a database based on the criteria identified from the query and the schema of the database to be searched. In some embodiments, the structured query is transmitted to another agent module or component to interact with one or more structured databases. For example, a user query of “how many patients are older than 18?” may be converted to an SQL query “SELECT COUNT(*) FROM demographic WHERE age>18.” FIG. 5B illustrates an example workflow for interacting with an agent in accordance with some embodiments. As shown in FIG. 5B, patient data 550 may be partitioned into a plurality of portions 552. In some embodiments, each portion may be comprised of data having a different data modality. For example, the portion 552-1 may consist of text data (e.g., structured and/or unstructured text data) and the portion 552-n may consist of image data (e.g., ultrasound images, x-ray images, and/or other types of images). In some embodiments, each portion in the portions 552 corresponds to a different period of time (e.g., a different day, week, or month). In some embodiments, each portion in the portions 552 corresponds to data obtained from a different source (e.g., from a different external database). In the example of FIG. 5B, each portion is converted into a set of chunks 554. In some embodiments, a set of chunks 554 is generated by summarizing the corresponding portion. In some embodiments, a set of chunks 554 is generated by annotating and/or labeling the corresponding portion. In some embodiments, a set of chunks 554 is generated by partitioning, summarizing, annotating, characterizing, and/or labeling the corresponding portion. In some embodiments, each chunk in the set of chunks is converted into an embedding (e.g., a vector embedding having 1 or more dimensions). In the example of FIG. 5B, information from the set of chunks 554 is stored in a vector store 556 (e.g., corresponding to one or more vector spaces). The vector store 556 may be an instance of the database 514 in FIG. 5A.

In accordance with some embodiments, a prompt template 560 is provided to an agent module 562 (e.g., a retrieval-augmented generative (RAG) agent module). The prompt template 560 provides instructions and/or parameters for providing query responses. For example, the prompt template 560 may indicate how to format, summarize, and/or support model outputs. In some embodiments, the prompt template 560 indicates what types of context information should be used to analyze and respond to user queries. FIG. 5B illustrates an example query 564 being provided to the agent module 562 along with context information 566. In some embodiments, the context information 566 is generated based on prior interactions with the user, a user profile associated with the user, one or more user preferences of the user, and/or a similarity analysis of the query 564. The agent module 562 in FIG. 5B provides an example response 570 (e.g., based on an output from one or more ML models, such as one or more LLMs) that is responsive to the query 564 and structured according to the prompt template 560.

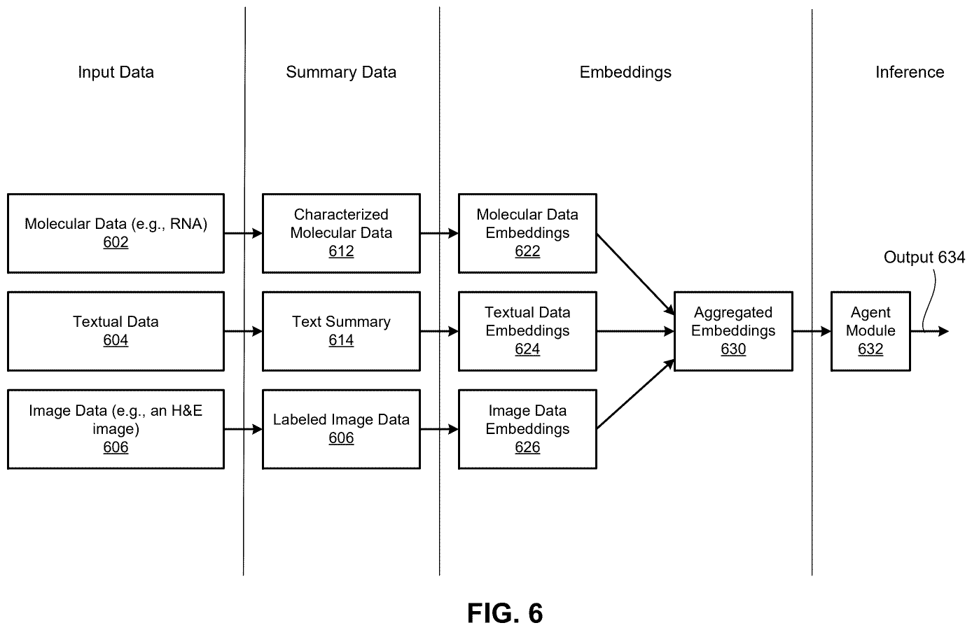

FIG. 6 illustrates an example process for using multi-modal data in an agent system in accordance with some embodiments. In the example of FIG. 6, multi-modal input data is obtained. The input data includes molecular data 602, textual data 604, and image data 606. In some embodiments, other data modalities are included, such as a structured text modality, an unstructured text modality, a tabular data modality, a data visualizations modality, one or more image modalities, an audio modality, a video modality, a biological sequence modality, a natural language modality, and/or a source code modality.

The input data is converted to summary data in FIG. 6. In some embodiments, a set of one or more agents is used to generate the summary data. In some embodiments, a different agent is used for each modality of data. In some embodiments, a single agent is used to summarize two or more modalities of data. For example, an ML model may be trained and prompted to generate summaries for a particular modality (or set of modalities) of data. In some embodiments, the summary data comprises human-readable data (e.g., data intended to be readily understood by a human reader). In some embodiments, a summarization agent is used to generate human-readable summaries for one or more data modalities. In FIG. 6, the molecular data 602 is converted to characterized molecular data. In some embodiments, the characterized molecular data is labeled and/or annotated (e.g., identifying regions of interest and associated molecular types). In some embodiments, characterizing the molecular data comprises identifying portions of the molecular data that are relevant (e.g., relevant to a corresponding inference/output 634), characterizing the relevant portions, and discarding other portions of the molecular data. The textual data 604 in FIG. 6 is converted to a text summary 614 (e.g., several pages are summarized in 1-2 paragraphs). In some embodiments, the text summary 614 is a concise version of the textual data 604 that highlights key points, main ideas, and/or other important information so as to provide an understanding of the textual data 604. The image data 606 in FIG. 6 is converted to labeled image data 606. In some embodiments, the labeled image data characterized and/or annotated (e.g., identifying features and objects in the image data). In some embodiments, labeling the image data comprises identifying portions of the image data that are relevant (e.g., relevant to a corresponding inference/output 634), characterizing the relevant portions, and discarding other portions of the image data.

In the example of FIG. 6, each modality of summary data is converted to a corresponding set of embeddings. In some embodiments, a set of one or more agents is used to generate embeddings. In some embodiments, a different agent is used for each modality of summary data. In some embodiments, a single agent is used to generate embeddings for two or more modalities of summary data. In some embodiments, embeddings are generated from summary data for a first data modality and embeddings are generated from input data (e.g., raw data) for second data modality. In some embodiments, the embeddings are vectors (e.g., feature vectors) having one or more dimensions and configured to be input into an ML model (e.g., input into a neural network). In some embodiments, the embeddings are configured to be in a same vector space (e.g., a vector space used by an ML model configured to answer user queries related to the input data). Generating embeddings from summary data as opposed to raw input data can reduce the size and dimensionality of the embeddings, which can reduce latency, processing overhead, and/or storage requirements. FIG. 6 shows the characterized molecular data 612 being used to generate molecular data embeddings 622, the text summary 614 being used to generate the textual data embeddings 624, and the labeled image data 606 being used to generate the image data embeddings 626. In some embodiments, the different types of embeddings have different dimensionalities. In some embodiments, at least a subset of the embeddings 622, 624, and 626 are pre-processed to reduce their dimensionality (e.g., such that all embeddings have a same dimensionality). For example, the aggregated embeddings 630 may be generated using equal length vector inputs for each modality. In some embodiments, the pre-processing is performed by a same agent that generates the corresponding embedding. In some embodiments, the pre-processing is performed by a different agent. In some embodiments, the embeddings 622, 624, and 626 are split into branches, each branch having a dimensionality that is less than (or equal to) a threshold length (e.g., smaller than any input dimension). In some embodiments, the branches are combined/grouped when generating the aggregated embeddings 630. In some embodiments, the splitting/branching is performed by a same agent that generates the corresponding embedding. In some embodiments, the splitting is performed by a different agent.

In the example of FIG. 6, the different types of embeddings are combined to generate aggregated embeddings 630. In some embodiments the aggregated embeddings 630 are generated by concatenating the molecular data embeddings 622, the textual data embeddings 624, and/or the image data embeddings 626. In some embodiments, the aggregated embeddings are not generated (e.g., the different types of embeddings are provided to the agent module 632 without being combined). In FIG. 6, the aggregated embeddings 630 are input to an agent module 632 (e.g., an instance of an agent module 226 or 316). In some embodiments, the agent module 632 includes one or more ML models. For example, the agent module 632 may include a multi-modal ML model configured to operate on molecular, textual, and image data. As shown in FIG. 6, the agent module 632 provides an output 634 based on the aggregated embeddings 630 in accordance with one or more prompts, requests, and/or queries.

In some embodiments, one or more of the embeddings are generated (e.g., before receiving the input data) and stored (e.g., in a database of the platform 100) for subsequent use (e.g., for use in generating the aggregated embeddings 630). In some embodiments, at least a subset of the embeddings 622, 624, and 626 are generated and stored (e.g., generated in an offline manner) prior to being used when generating the aggregated embeddings 630. In some embodiments, the embeddings 622, 624, and 626 are generated (e.g., generated in an online manner) and used for generating the aggregated embeddings 630 (e.g., generated and used on demand).

As an example, an agent module may be configured to analyze clinical information identifying a line of therapy given to patients to output a corresponding recommendation. To select candidates, one or more guidelines may be used to select a pool of drugs for ranking. The guidelines may include compliance guidelines. The agent module may include a transformer model, or other type of model configured to analyze multi-modal data (such as DNA data, RNA data, genomic data).

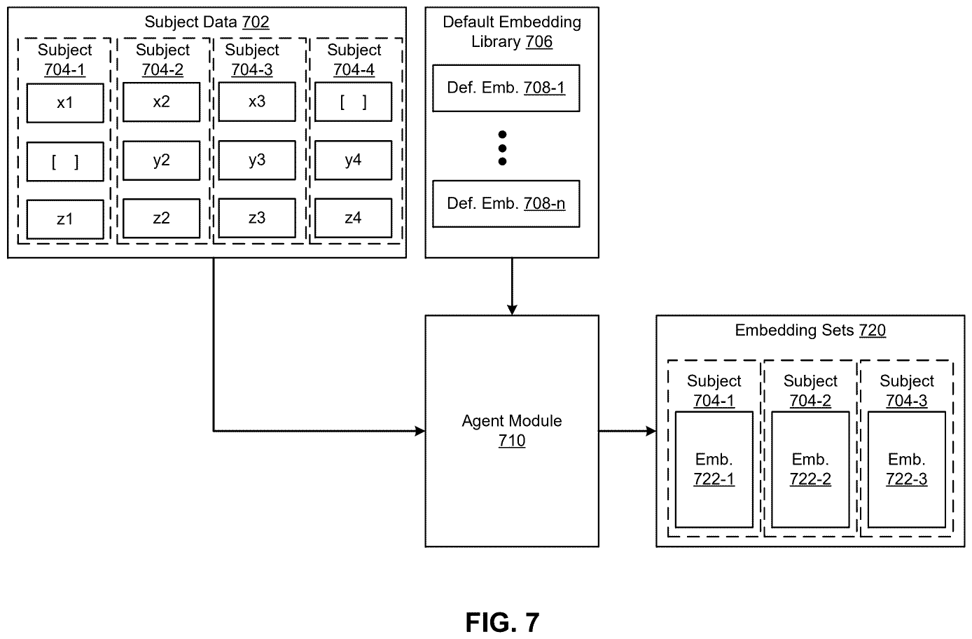

FIG. 7 illustrates an example process for using multi-modal data with missing modalities in accordance with some embodiments. In FIG. 7, subject data 702 (e.g., respective EHRs for a set of patients) is converted to embedding sets 720. In accordance with some embodiments, a predefined set of modalities (e.g., a set of n modalities, where n is a positive integer) are used for each set of subject data. As an example, if the subject data for a particular subject is missing one of the data modalities (e.g., a particular patient has no ultrasound data, x-ray data, and/or molecular data) then a default embedding is used for the missing data modality. In the example of FIG. 7, the subject data 702 for a subject 704-1 is missing a ‘y’ modality of data (e.g., image data or molecular data). In this example, a default embedding 708 (e.g., the default embedding 708-1) is used to fill the missing ‘y’ modality of data. In some embodiments, the default embedding library 706 includes a default embedding for each modality of data in the predefined set of data modalities. FIG. 7 also shows the subject data 702 for a subject 704-4 missing an ‘x’ modality of data (e.g., biological data, audio data, or a particular type of imaging data). A default embedding (e.g., the default embedding 708-n) may be used to fill the missing ‘x’ modality of data. In accordance with some embodiments, the agent module 710 (e.g., an instance of an agent module 226 or 316) is configured to generate embedding sets 720 from the subject data 702. The agent module 710 may also be configured to summarize the subject data 702 before generating the embedding sets 720. In some embodiments, the subject data 702 is summarized data (e.g., has already been summarized by a different component or system). In some embodiments, the agent module 710 is configured to identify missing modalities of data and obtain default embeddings 708 from the default embedding library 706 to compensate for the missing modalities. In some embodiments, the default embeddings are assigned a reduced weight (e.g., a zero weight or a weight that is significantly lower than weights assigned to the subject data 702). In this way, the embedding sets 720 are generated for the subjects 704 and do not have any missing modalities (e.g., each embedding 722 in the embedding sets has a same dimensionality).

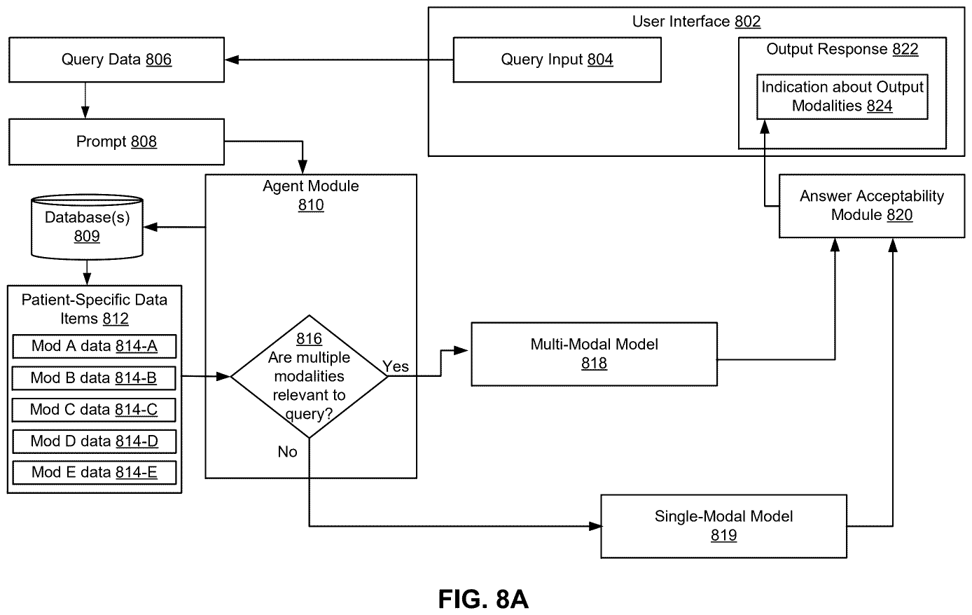

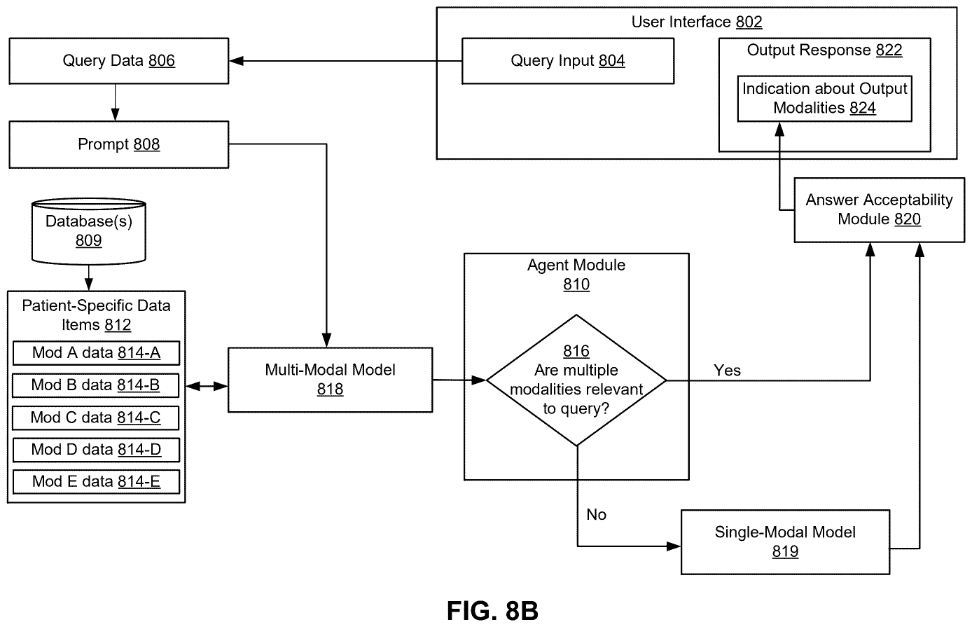

FIG. 8A illustrates an example process for identifying important modalities from a multi-modal analysis in accordance with some embodiments. In FIG. 8A, a user interface 802 is presented to a user. For example, the user interface 802 may correspond to the platform 100. A query input 804 is received via the user interface 802. For example, the user may type in or speak the query. In some embodiments, the query input 804 is a natural language query (e.g., in a conversational tone). In accordance with some embodiments, query data 806 is identified from the query input 804 (e.g., key terms, data points, and/or concepts may be identified from the query). In some embodiments, an agent module (e.g., a query agent) is used to identify the query data 806 from the query input 804. In accordance with some embodiments, the query data 806 is incorporated into a prompt 808 (e.g., along with context information and/or one or more preset prompt instructions). In some embodiments, an agent module (e.g., a query agent) is used to generate the prompt 808. In accordance with some embodiments, an agent module 810 (e.g., a modality determination agent) receives the prompt 808. The agent module 810 may request data from the database(s) 809 (e.g., an instance of the databases 350) that is relevant to the prompt 808 (e.g., the patient-specific data items 812). The patient-specific data items 812 may include one or more modalities of data (e.g., the modalities 814 shown in FIG. 8A). In some embodiments, the patient-specific data items 812 are obtained from the database(s) 350. In accordance with some embodiments, the agent module 810 is configured to determine (816) whether one or multiple modalities of data are relevant to the prompt 808. For example, the agent module 810 may determine which modalities of data are relevant based on the type of question/request being asked (such as “does the ultrasound image show any irregularities?” or “summarize the doctor's written notes from the last 5 visits”). In these examples, the question/request specifies the relevant data modalities. As another example, the question/request may indicate multiple modalities are relevant (e.g., “do the x-ray images indicate anything different from the other parts of the record?”). In some embodiments, the agent module 810 determines which data modalities may be relevant based on prior training (e.g., training on prompts and corresponding responses).

In accordance with a determination that multiple modalities of data are relevant to the prompt 808 (e.g., the modalities (mods) 814-A through 814-E), the agent module 810 provides the prompt 808 (and optionally the patient-specific data items 812) to a multi-modal model 818. In some embodiments, the multi-modal model 818 is a component of different agent (e.g., a multi-modal analysis agent). In accordance with a determination that a single modality of data is relevant to the prompt 808 (e.g., the modality 814-B), the agent module 810 provides the prompt 808 (and optionally the patient-specific data items 812) to a single-modal model 819. For example, the single-modal model 819 may be a model that is trained on and/or prompted to use a particular data modality. In some embodiments, the single-modal model 819 is a component of different agent (e.g., a modality-specific agent).

In some embodiments, the prompt 808 is provided to the multi-modal model 818 and the single-modal model 819 in accordance with a determination that multiple modalities may be relevant to the prompt 808, but a single modality is most significant (e.g., has a highest relative weight). In some embodiments, the prompt 808 is provided to the multi-modal model 818 in accordance with a determination that no modality has a relevance rating above a first predetermined threshold (e.g., greater than 80%, 90%, or 95%). In some embodiments, the prompt is provided to a single-modal model 819 in accordance with a determination that the corresponding data modality has a relevance rating above a second predefined threshold (e.g., 60%, 70%, or 80%). In some embodiments, the agent module 810 determines the relevant ratings based on prior training (e.g., training on prompts and corresponding responses).

The determining (816) may be performed in various manners. For example, the system may utilize a lookup table or other form of list associating different agents with a set of modalities. In another example, the system may utilize a relevance rating (e.g., where a rating is determined based on the variance of the underlying features or embeddings of a modality in association with the predicted values from an agent) where the more variance across an embedding tied to a decision difference increases the rating. In some embodiments, the relevance rating may be determined using a feature importance approach. In another example, the system may utilize a comparison between model performances on different modalities of data. For example, a plurality of models may be trained and operated using each modality, each pairwise selection of modalities, each triplet selection of modalities, each quadruplet selection of modalities, and so forth (or a subset thereof). In some embodiments, the training continues until a best performing model is determined. In another example, the system may include multiple of the previously-mentioned approaches, such as where a list is created from a feature importance model trained on all modalities and the list includes which modalities contributed more than a threshold's importance to the outcome and so forth. The threshold may be determined manually, automatically based on a best increased performance of the operating curve (e.g. where a new modality no longer results in a significant improvement over the previous performance), or using other autonomous methods.

In accordance with some embodiments, the output of the multi-modal model 818 and/or the single-modal model 819 is provided to an answer acceptability module 820. The answer acceptability module 820 may correspond to a different agent (e.g., an output analysis agent). The answer acceptability module 820 is configured to determine whether the output from the multi-modal model 818 and/or the single-modal model 819 meets one or more criteria. In some embodiments, the criteria are specific to a particular type of output (e.g., different criteria are used for an output about a treatment plan than are used for an output about adverse effects for a particular medication). In some embodiments, the answer acceptability module 820 is configured to format the model outputs (e.g., convert the model outputs to a natural language (e.g., conversational) output). In some embodiments, the answer acceptability module 820 is configured to combine outputs from multiple models into a single response for the user. In accordance with some embodiments, the answer acceptability module 820 is configured to provide an output response 822 that indicates which modalities were used to provide the output response 822 (e.g., which modalities were used by the multi-modal model 818 and/or the single-modal model 819 to generate the respective outputs). In some embodiments, the indication 824 about the modalities indicates which modalities were used to generate the output response 822. In some embodiments, the output response 822 includes an indication of which models were used to generate the response (e.g., the multi-modal model 818 and/or the single-modal model 819).

FIG. 8B illustrates another example process for identifying important modalities from a multi-modal analysis in accordance with some embodiments. The components in FIG. 8B are arranged differently from FIG. 8A. In FIG. 8B, the multi-modal model 818 receives the prompt 808 and generates a corresponding output. In some embodiments, the corresponding output includes an indication of which modalities were used to generate the output. In the example of FIG. 8B, the output of the multi-modal model 818 is analyzed by the agent module 810 to determine whether multiple data modalities were used to generate the output. As described previously, the determining (816) may utilize various approaches, including using a lookup table, a relevance rating, and/or model comparison. In some embodiments, the agent module 810 is configured to determine a relative contribution of each data modality. In accordance with a determination that multiple data modalities were used, the agent module 810 provides the output to the answer acceptability module 820. In accordance with a determination that a single data modality was used (or that a modality had a relevance score that is above a predetermined threshold), the agent module 810 provides the prompt 808 (and optionally the output of the multi-modal model 818) to the single-modal model 819 (e.g., to confirm the answer provided by the multi-modal model 818). For example, the multi-modal model 818 indicates that a text modality was the most relevant to its output, and the prompt 808 is then provided to the single-modal model 819. In this example, the answer acceptability module 820 may be configured to generate the output response 822 based on the outputs from the multi-modal model 818 and the single-modal model 819. In some embodiments, the answer acceptability module 820 merges the answers from the two models. In some embodiments, the answer acceptability module 820 selects the model output having the highest associated confidence level. In some embodiments, the answer acceptability module 820 prioritizes the output from the single-modal model 819 when the agent module 810 provides the prompt to the single-modal model 819.

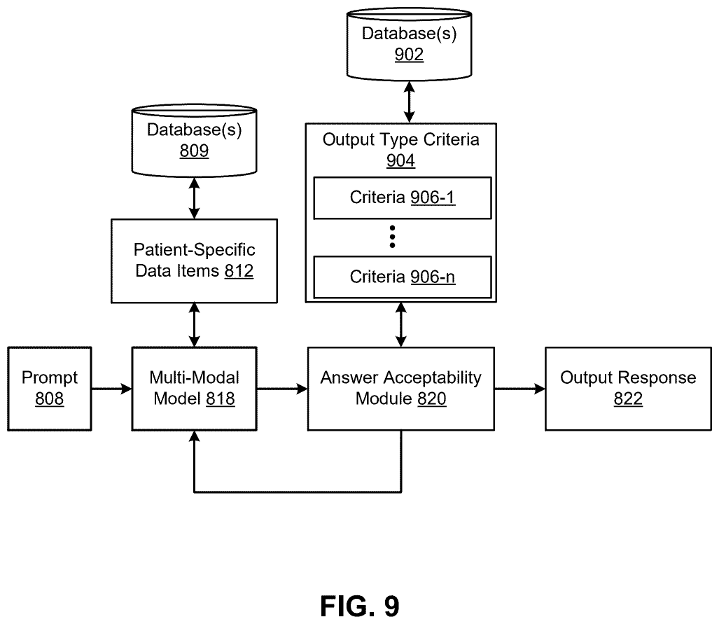

FIG. 9 illustrates an example process for applying type-specific criteria to model outputs in accordance with some embodiments. In FIG. 9, the prompt 808 is provided to the multi-modal model 818 and the multi-modal model 818 obtains the patient-specific data items 812 from the database(s) 809. In some embodiments, the prompt 808 is provided to the multi-modal model 818 via an input module (e.g., the user interface module 234 and/or the interface module 230). In various embodiments, the patient-specific data items 812 may be obtained before, in conjunction with, or after the prompt 808. The multi-modal model 818 produces a response output for the prompt 808 and the response output is provided to the answer acceptability module 820. In some embodiments, the multi-modal model 818 is replaced with a different model (or agent) that is configured to provide multiple types of responses.

In the example of FIG. 9, the answer acceptability module 820 evaluates the response output based on output type criteria 904 obtained from a database 902. In accordance with some embodiments, the output type criteria 904 include different sets of criteria 906 for different types of output (e.g., based on the type of subject matter in the output). As an example, the criteria 906-1 may correspond to an output regarding a treatment plan whereas the criteria 906-n may correspond to an output about a particular type of medication. In some embodiments, the database 902 includes sets of criteria for each response type available in the platform (e.g., response types the platform is configured to produce). In accordance with a determination that the response output from the multi-modal model 818 meets the type-specific criteria, the answer acceptability module 820 generates the corresponding output response 822. In some embodiments, in accordance with a determination that the response output from the multi-modal model 818 does not meet the type-specific criteria, the answer acceptability module 820 provides the feedback to the multi-modal model 818 to provide an updated response. In some embodiments, in accordance with a determination that the response output from the multi-modal model 818 does not meet the type-specific criteria, the answer acceptability module 820 provides a notification to the user (e.g., via the output response 822) that the prompt 808 could not be answered. In some embodiments, the notification includes a request for additional information from the user (e.g., needed to generate an acceptable response). In some embodiments, in accordance with a determination that the response output from the multi-modal model 818 does not meet the type-specific criteria, the answer acceptability module 820 provides information about the prompt 808 and/or the output from the multi-modal model 818 to a different model or agent (e.g., so that the different model or agent may update/correct the output).

Other example output types include current diagnoses, future diagnoses (e.g., within given time windows), disease state severity, disease progression or remission, survivorship (e.g., overall survivorship, progression-free survival), treatment responses (e.g., adverse or favorable). Other example output types include which treatment options are available and which clinical trials are available. Other example output types include whether increased monitoring is needed/suggested, whether care gaps exist, whether a pathology image shows a biomarker, where in an image certain tissue types exist, where in an image certain cell types exist, where in an image tumor infiltration is occurring, where in an image excess tissue is detected, and the tumor content of an image. Other example output types include what organs/bones are present in a radiological image, what regions of interest exist in a radiological image, what may be present in the regions of interest, and what diagnosis may be implicated by what is present in the regions of interest. Other example output types include what is different between two (consecutive) radiological images, what impact does the difference have on the subject's disease state, what corresponding treatment options are available, and how do similar patients respond to the treatment options. In some embodiments, an output includes two or more of the output types listed above (e.g., comprises a compound question). The system may provide output types other than the example type listed above, such as other outputs relating to disease states (e.g., in the areas of mental health, endocrinology, and cardiology), medication, treatment, clinical trials, research, and/or communication (e.g., filling out forms or drafting letters).

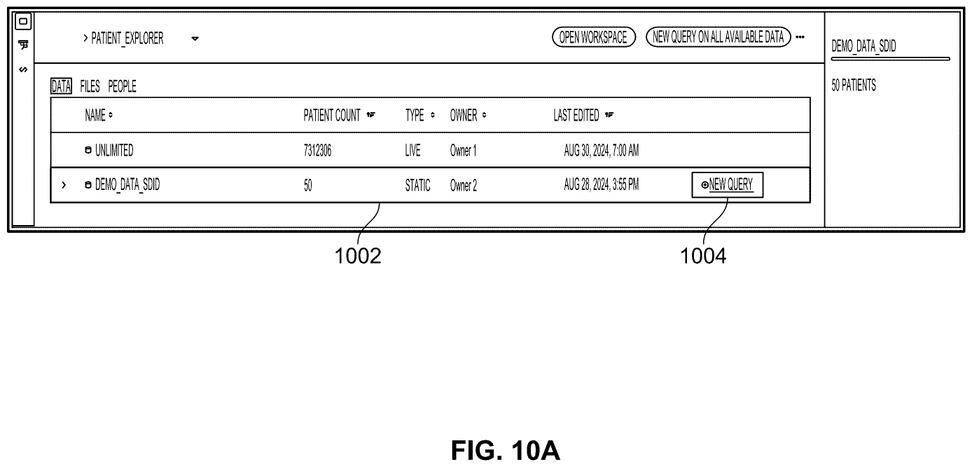

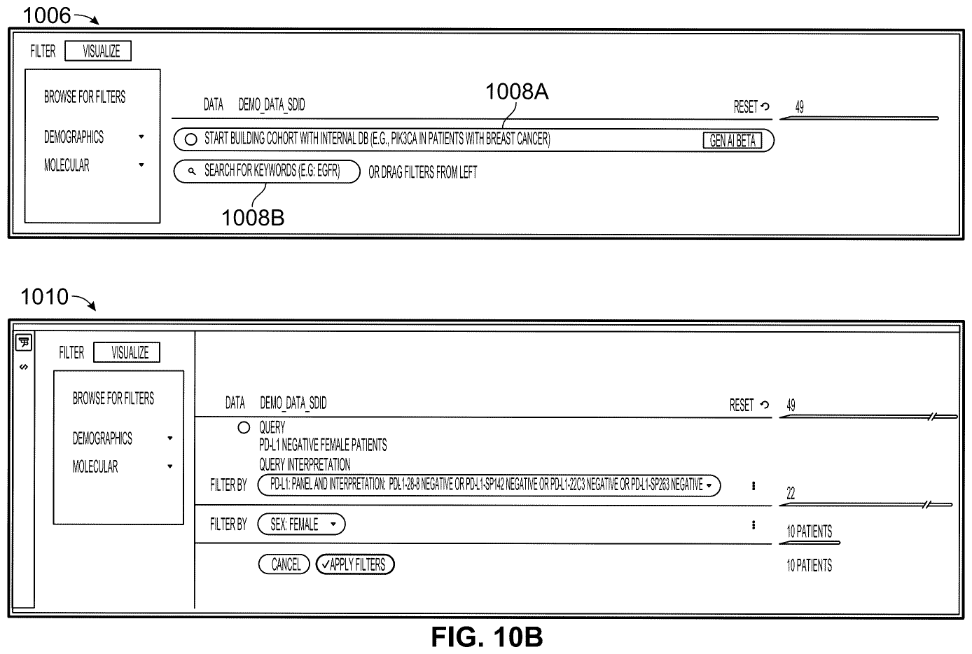

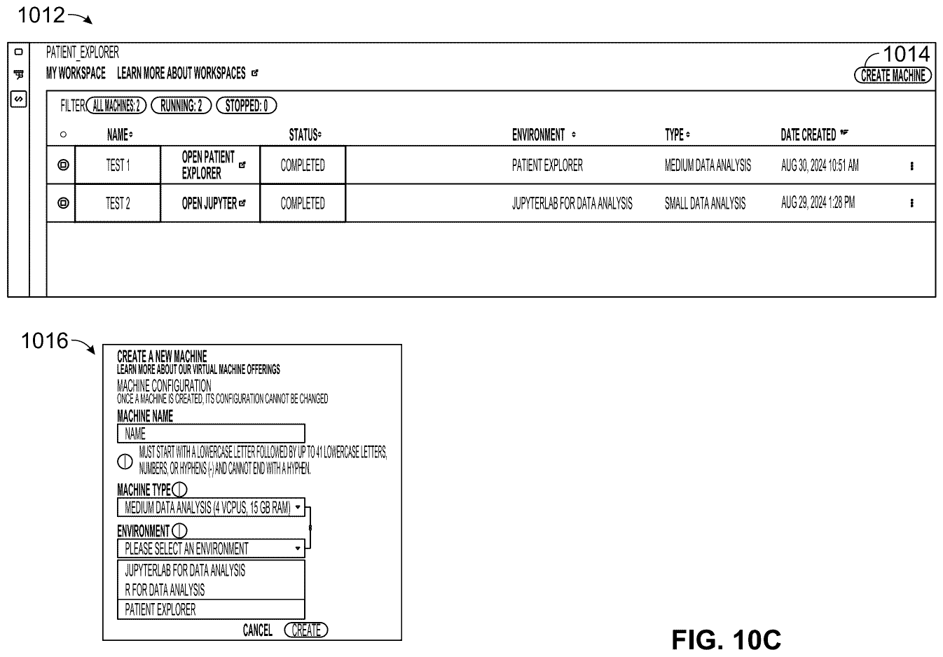

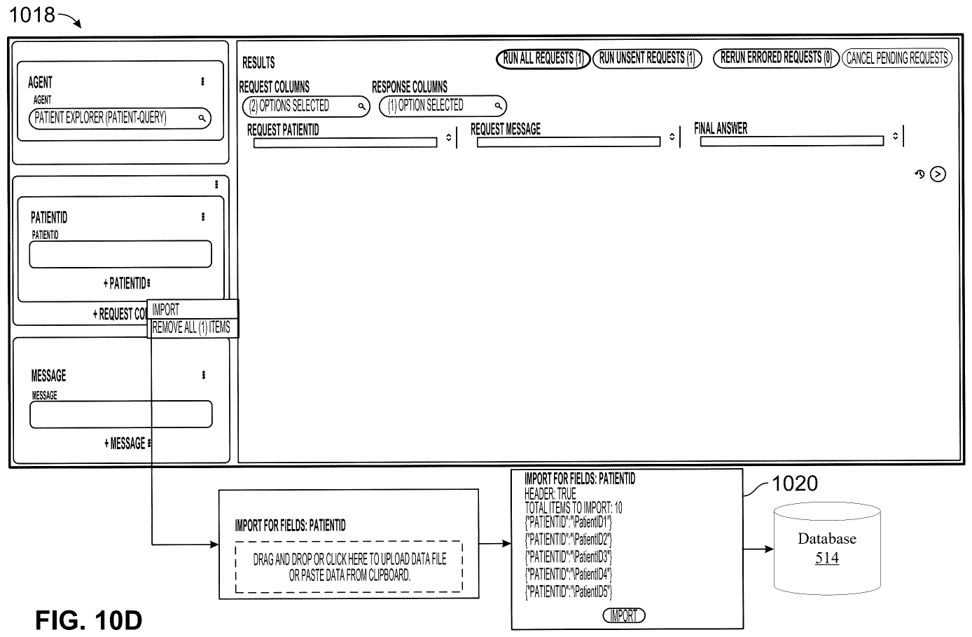

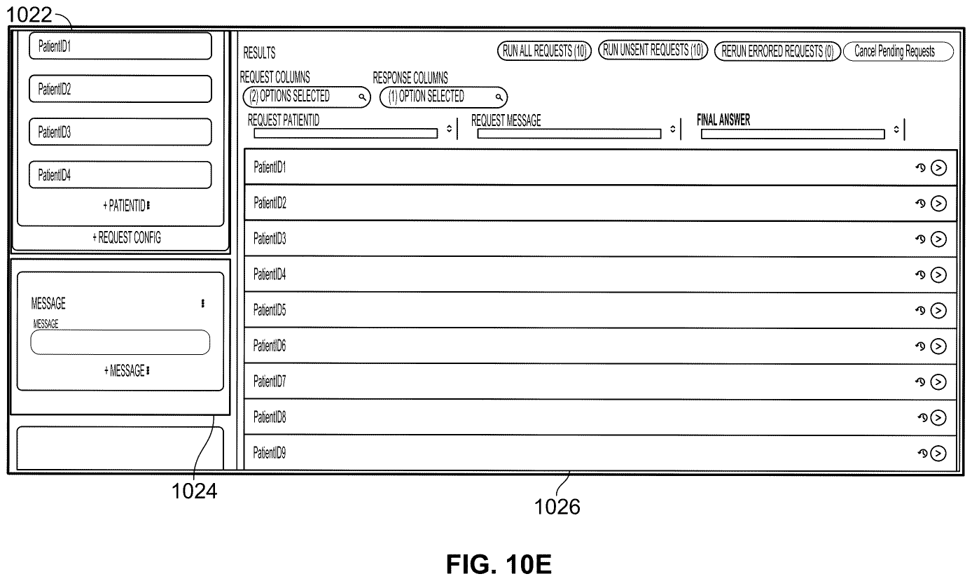

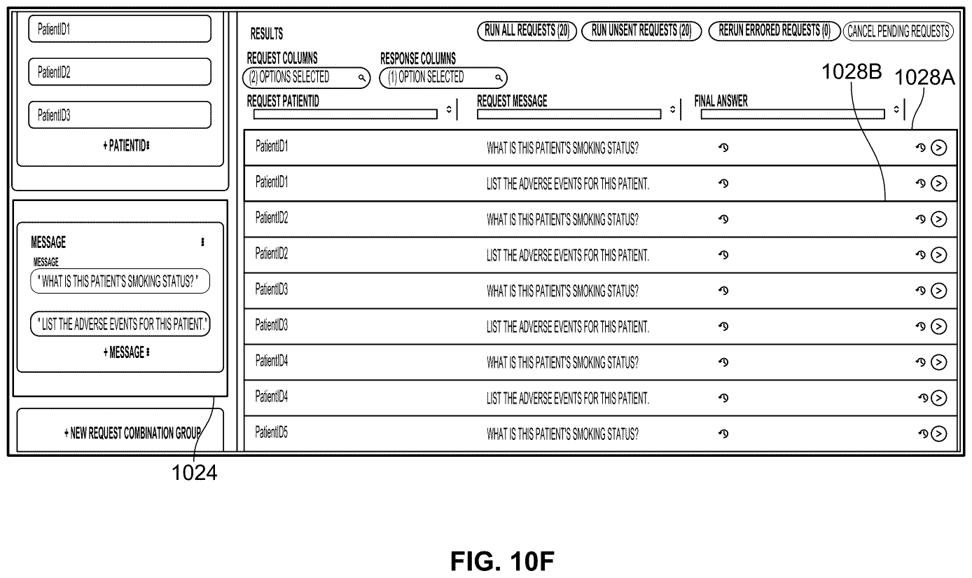

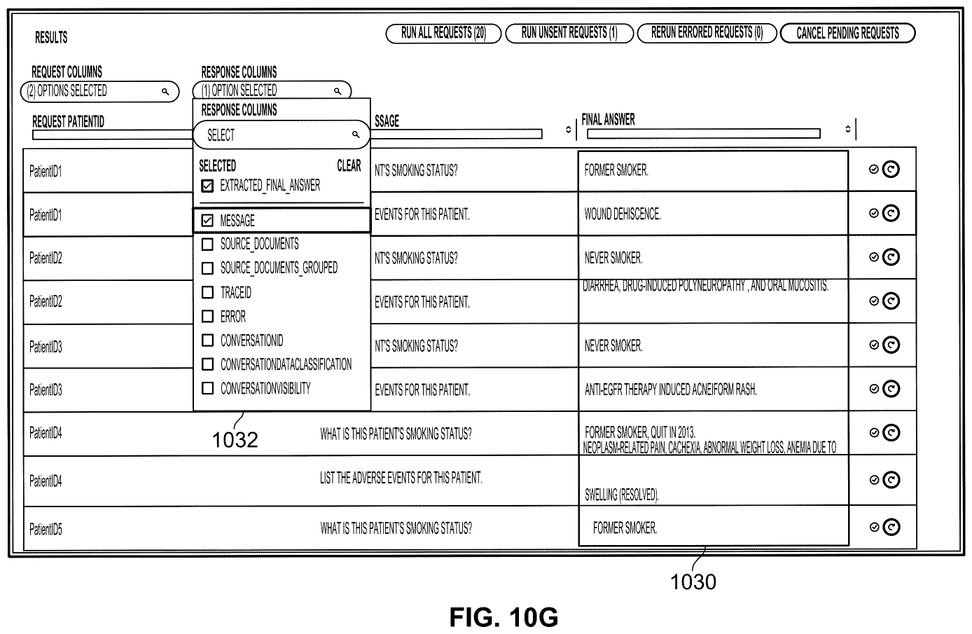

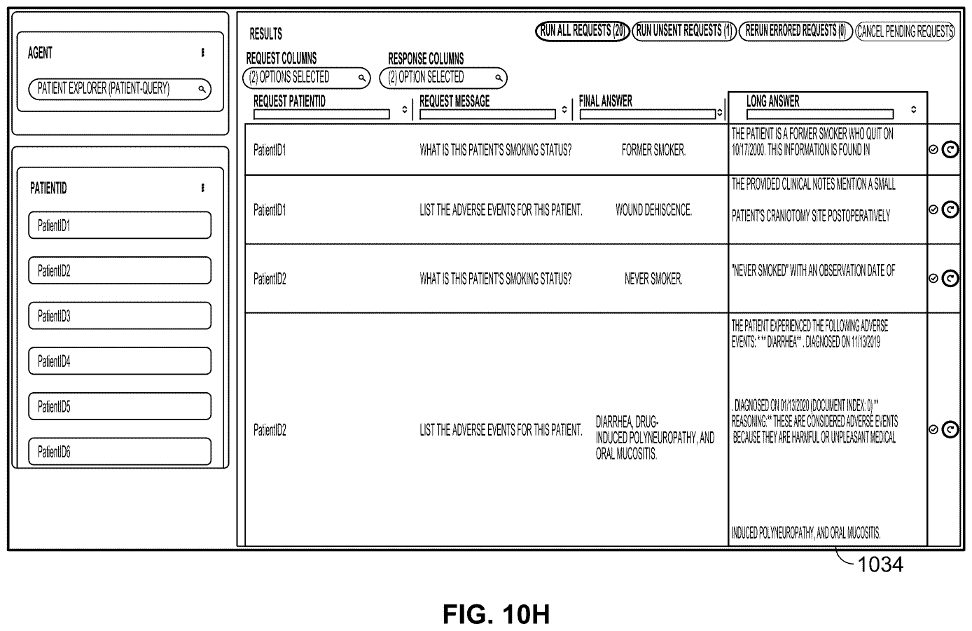

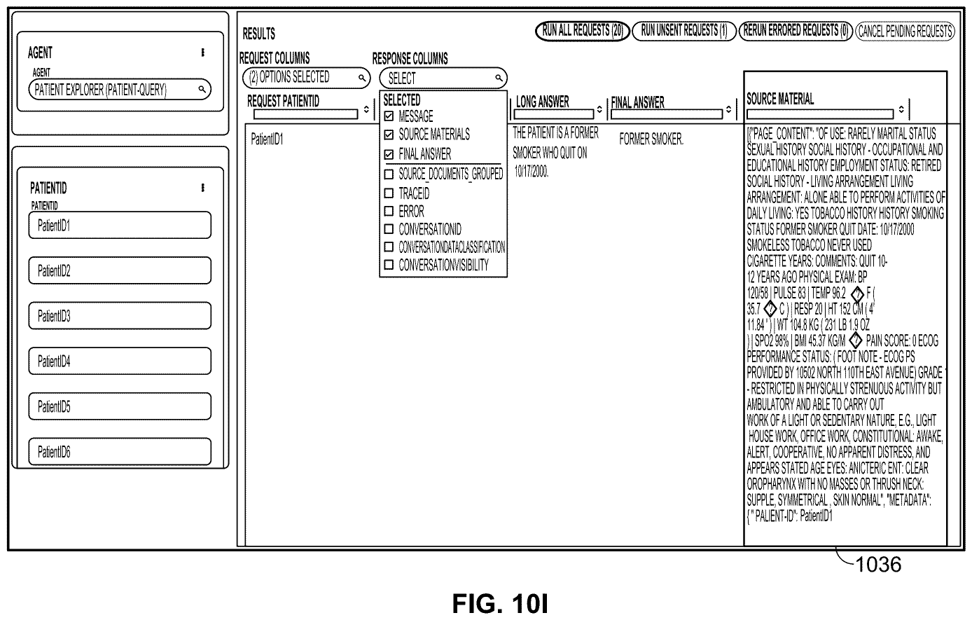

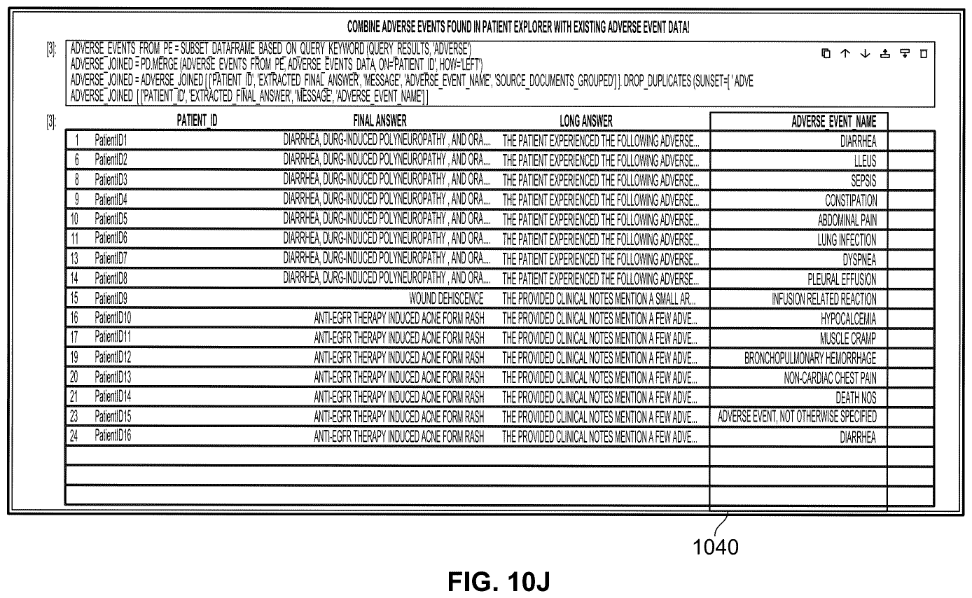

FIGS. 10A-10J illustrate example user interfaces and interactions for importing and querying subject data in accordance with some embodiments. In accordance with some embodiments, the user interfaces described with respect to FIGS. 10A-10J are part of a platform for using orchestrations, agent modules, and/or agent tools (e.g., the platform 100), which may be presented to a user as console of a web or desktop application (e.g., at a display of the client device 102).

FIG. 10A illustrates a first user interface, which may be a user interface of a web or desktop application associated with the platform 100, in accordance with some embodiments. In FIG. 10A, a user interface element 1002 is selected, the user interface element 1002 corresponding to a source dataset comprising a listing of structured patient data. For example, external data from an external database 108 as discussed previously with respect to FIG. 1. In some embodiments, the source dataset comprises multiple modalities of data. For example, the source dataset may comprise a multi-modal EHR for each patient in a set of patients. The data represented by the user interface element 1002 may be data that has not previously been stored in the one or more databases of the platform 100. In some embodiments, the user interface element 1002 is associated with a data plane that is separate from a control plane associated with the platform (e.g., where various agent configurations of the agent library 250 are stored).

FIG. 10A shows a user selecting a user interface element 1004 to perform a new query using the patient data associated with the user interface element 1002. In some embodiments, selection of the user interface element 1004 causes a series of operations to prepare an environment for performing the subsequent operations and presenting the user interfaces as illustrated in FIGS. 10B to 10J. In some embodiments, one or more orchestrations are selected to facilitate performance of the patient analysis on the patient data (e.g., one or more agent modules of the agent library 250). In some embodiments, the patient data is used to generate embeddings associated with the source dataset, including the listing of patient data, which may be stored within a vector space associated with the healthcare management application (e.g., the vector store 556).

FIG. 10B shows another set of user interfaces that include user interface elements which allow for a user to identify a cohort of patients from the listing of patient data within the source dataset discussed above with respect to FIG. 10A. A cohort builder user interface 1006 includes respective textual fields 1008A and 1008B, which enable a user to input a textual prompt, which may be supplied to an agent or agent module in order to determine a filter to apply to the patient data, thereby identifying a subset of the patient data corresponding to a desired cohort (e.g., for the user to analyze further). In some embodiments, the user interface 1006 includes additional fields and/or other types of fields (e.g., drop-down menus, radial buttons, etc.) not shown in the example of FIG. 10B.

FIG. 10B also shows a user interface element 1010 that represents a set of filters to be applied to the listing of patient data to identify a portion of the source dataset corresponding to the plurality of subjects (e.g., based on user inputs to the cohort builder user interface 1006, such as a textual prompt input into the field 1008A). In some embodiments, the application is configured to apply the textual prompt input by the user to an ML model (e.g., an LLM of an agent module) which can be used to determine a textual prompt to be applied to the patient data as a filter. For example, a query for “pd-11 negative female patients” could be interpreted as a filter that could be applied to the patient data (e.g., “PD-L1: Panel and Interpretation: pd-11-28-8 Negative or pd-11-sp142 Negative or pd-11-sp142 Negative or pd-11-22c3 Negative or pd-11-sp263 Negative”).

FIG. 10C illustrates another set of user interfaces that enable a user to create a workspace (e.g., a virtual machine corresponding to the source dataset and/or the cohort of patients selected from the source dataset in FIG. 10B) within the platform 100 (e.g., a patient explorer workspace), in accordance with some embodiments. A user interface 1012 includes a user interface element 1014 to create a workspace (e.g., “Create Machine”). In some embodiments, each respective workspace created by the user is associated with a different virtual machine, which may be specifically configured for the task that the user intends to perform within the workspace. For example, a set of agent modules may be selected from the agent library 250 based on information in the source dataset. The use of a different virtual machine provides advantages in effecting data governing compliance.

FIG. 10C also shows a user interface element 1016 (e.g., a workspace configuration user interface) that can be presented after the user provides the user input at the user interface element 1014, and that includes user interface elements that enable the user to configure the settings of the workspace that they are creating. For example, the user can provide a name for the workspace, specify a machine type for the workspace, and select an environment type from a selectable list of options (e.g., “JupyterLab for data analysis”; “R for data analysis”; and “Patient Explorer”). In some embodiments, other configurable settings are presented to the user that are not shown in FIG. 10C (e.g., a duration for the workspace to run for before auto-stopping, a type of orchestration, agent module, or ML model to use as a default for the workspace, etc.). By providing for separation between different datasets within the system, the techniques described herein can provide for more robust data privacy with respect to, for example, patient health data.

FIG. 10D shows another user interface 1018 (e.g., a patient explorer workspace user interface) that represents the workspace that was initialized in FIG. 10C in accordance with some embodiments. In FIG. 10D, a user input is directed to a user interface element for importing data, which results in the patient data that was selected in FIG. 10A being imported to the patient explorer workspace. In some embodiments, in accordance with the user selecting patient data to import, the selected patient data is used to generate embeddings, which can be stored in an embedding space (e.g., a vector space), such as a vector space within the database 514 for further use with the patient explorer module. In some embodiments, the import process includes summarizing the patient data and/or generating embeddings for the patient data. In some embodiments, the plurality of embeddings is generated using the techniques described previously, e.g., with respect to FIGS. 5A and 5B. For example, the selected patient data may undergo operations similar to those performed on the imported data 506 in FIG. 5A (e.g., summarizing, chunking, snippetizing, and/or otherwise partitioning the data). In some embodiments, patient names (and other PII) are de-identified and/or replaced with patient identifiers (e.g., the patient identifiers shown in the user interface element 1020).

FIG. 10E shows the patient explorer user interface 1018 after importation of the source data (e.g., based on the user inputs illustrated in FIG. 10D). The user interface in FIG. 10E includes a patient-listing user interface element 1022 showing which patients are in the selected cohort. The patient explorer user interface further includes a request prompt user interface element 1024 to which the user can provide textual inputs to in order for the prompts to be applied to the patients within the cohort. The patient explorer user interface in FIG. 10E also includes a request table 1026 that lists user queries requested by the user, e.g., via the textual inputs provided to the request prompt user interface element. In accordance with some embodiments, the patient explorer user interface includes a user interface element for initiating each user query listed in the request table 1026, and a plurality of user interface elements (e.g., within each respective row of the request table 1026) for initiating individual requests for respective user queries associated with the respective rows of the request table 1026.

FIG. 10F illustrates another user interface of the patient explorer (e.g., after the user has provided two different requests into the request prompt user interface element 1024). In accordance with some embodiments, when a user adds a message to the request prompt user interface element 1024, a row in the results field is added for each patient imported from the patient data, where the respective row is associated with that particular message prompt. For example, the request table 1026 includes a first row 1028A corresponding to a first patient identifier and the first user query in the request prompt user interface element 1024 (e.g., “What is this patient's smoking status?”), and a second row 1028B corresponding to the first patient identifier and the second user query in the request prompt user interface element 1024 (e.g., “List the adverse events for this patient”). In accordance with some embodiments, each row corresponds to a request to a particular task-specific orchestration (e.g., comprising an LLM and/or other ML model) based on the (i) the data associated with the respective patient ID, and (ii) the query (e.g., request message). In some embodiments, a template may be used to create a request to the task-specific orchestration based on the combination of the individual items (e.g., the user query). In accordance with some embodiments, a task-specific orchestration selected for running the user query corresponds to a different agent module than the orchestration that was used to apply filters to the listing of patients as described in FIG. 10B. In some embodiments, the task-specific orchestration is selected based on the content of the query.

FIG. 10G shows the patient explorer user interface after the request to the task-specific orchestration has been executed. As shown in FIG. 10G, a column 1030 of the request table 1026 includes responses (e.g., short answers) from the task-specific orchestration for each of the respective requests described in FIG. 10F. FIG. 10G also shows a user interface element 1032 for adjusting which columns of information are shown to the patient explorer user interface.

FIG. 10H shows the patient explorer user interface including a full response message (e.g., a long answer) in addition to the final answer (e.g., the short answer) for each patient and query combination (e.g., after the user has selected to show a detailed response message from the task-specific orchestration based on each respective request). For example, the task-specific orchestration can be configured to provide two different responses, one that includes a short phrase directly responding to the user's request, and another response that provides a detailed explanation as to how the task-specific orchestration determined the response. For example, in response to the user input directed to the response message option within the user interface element 1032, the request table 1026 may be modified to include a column 1034 including detailed information about how the task-specific orchestration determined the final answer for the respective patient-directed prompt associated with each row. In some embodiments, the user interface includes one or more of: a final answer, a long answer, an indication of the corresponding source material, an indication of the corresponding source data modalities, an indication of a confidence level for the response, and an indication of the machine-learning models used to generate the response(s).

FIG. 10I shows the patient explorer user interface including a source documents column in addition to the final answer for each patient and query combination (e.g., after the user has selected another option to cause a different column to be presented as part of the patient explorer user interface). In accordance with some embodiments, a listing of source documents is presented for each respective patient-directed prompt, as shown by the column 1036 in FIG. 10I.

FIG. 10J shows another user interface to combine the patient data that was imported into the patient explorer workspace with other data stored in the healthcare management system. This enables a user to load the data that was generated in the patient explorer workspace based on the patient data, and can combine it with a different dataset within a database associated with the healthcare management application (e.g., the adverse events data represented by column 1040). In some embodiments, the user can use data generated in one workspace within a different workspace, including a different workspace having a different working environment. FIG. 10J shows that data from the source dataset (e.g., as described with respect to FIGS. 10A-10I) may be combined with data from another dataset (e.g., data from an internal database, such as the system database(s) 350).

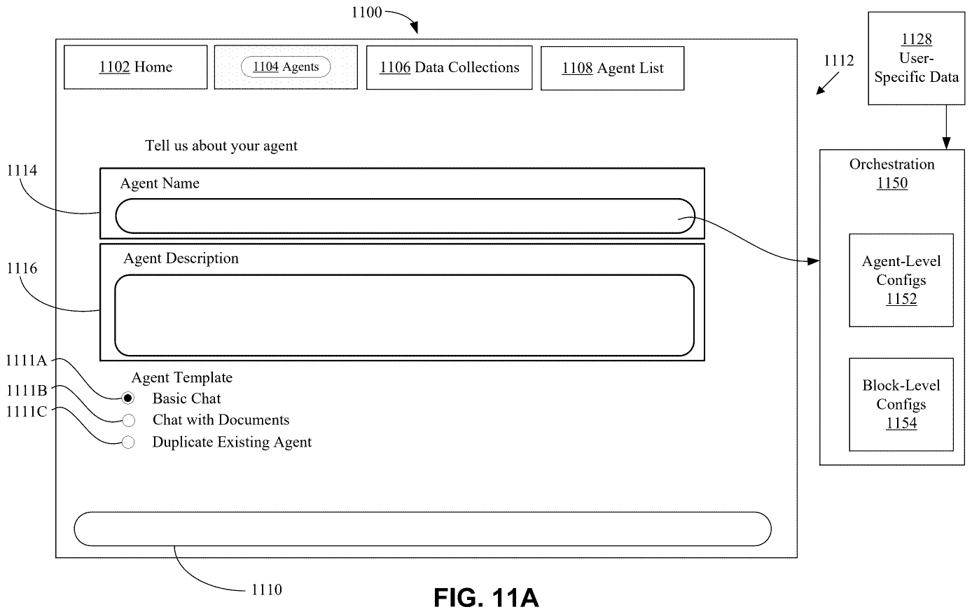

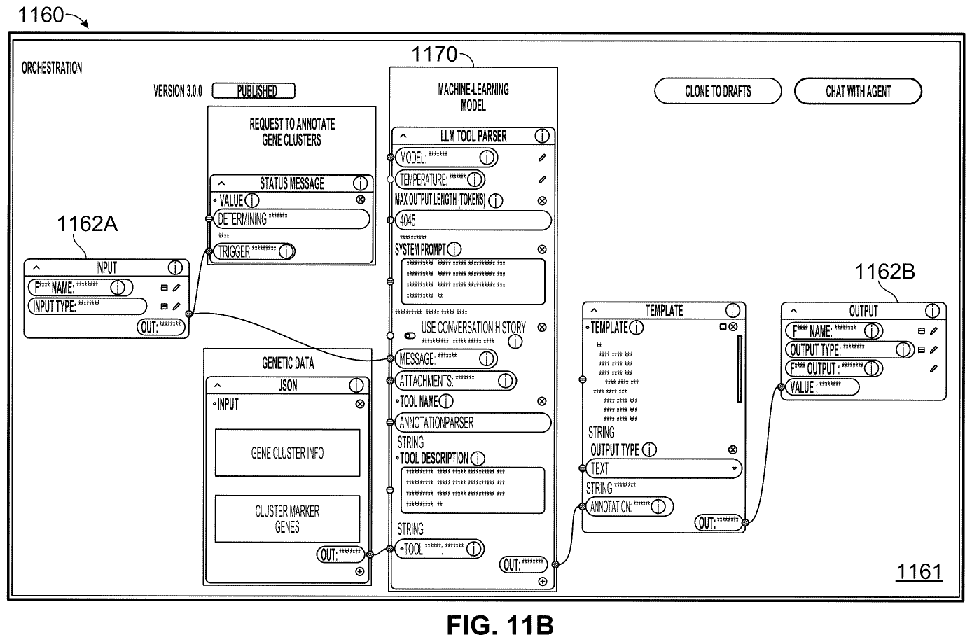

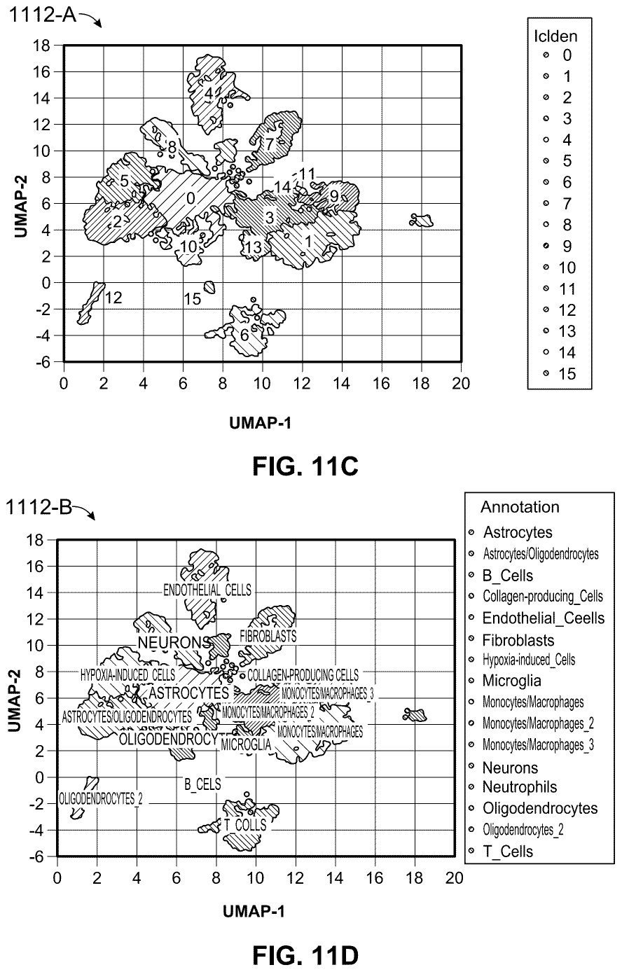

FIG. 11A shows a user interface 1112 of an agent-builder application 1100 in accordance with some embodiments. The agent-builder application 1100 may include various user interface elements for causing operations to modify respective orchestrations associated with the user of the agent-builder application 1100. In accordance with some embodiments, a user is permitted access to the agent-builder application 1100 by providing user credentials, e.g., from the client device 102 to the server system 106. The user interface 1112 includes a form-builder user interface element 1114 for interacting with (e.g., instantiating and/or configuring) an agent module (e.g., an agent module 226 or 316) in accordance with some embodiments. In some embodiments, the user interface 1112 includes global user interface elements that are present within different respective user interfaces of the agent-builder application, as described herein. For example, the user interface 1112 includes respective user interface elements for accessing different user interfaces of the agent-builder application 1100 (e.g., a user interface element 1102 for accessing a home user interface, a user interface element 1104 for accessing an agent-builder user interface, a user interface element 1106 for accessing a data viewing user interface, and a user interface element 1108 for viewing a list of task-specific orchestrations (e.g., task-specific agents) that are available to the user accessing the agent-builder application 1100. For example, the global user interface elements may include a prompt user interface element 1110 for initiating a chat session with a respective agent module of the agent-builder application 1100.

The user interface 1112 includes a plurality of user interface elements for modifying an orchestration 1150 (e.g., a task-specific orchestration, which may comprise an agent module and/or an agent architecture) in accordance with some embodiments. For example, the user interface 1112 includes a user interface element 1114 for naming the orchestration 1150, and a user interface element 1116 for providing a description of the orchestration. In some embodiments, other users having access to the data associated with the orchestration 1150 may access and/or implement the orchestration by selecting it from an agent library (e.g., the agent library 250). In accordance with some embodiments, the user interface 1112 also includes a template-selector section for interacting with a plurality of user interface elements corresponding to different default orchestrations that the user can select to provide an initial node architecture 254 to the orchestration 1150 (e.g., a user interface element 1111A for creating a task-specific orchestration for interacting with a general-purpose machine-learning model, a user interface element 1111B for interacting with a task-specific orchestration that includes a machine-learning model (e.g., a general-purpose machine-learning model and/or a task-specific machine learning model) that has been trained with specific data (e.g., from a data collection that is continuously updated in real-time), and a user interface element 1111C for interacting with a task-specific orchestration that was previously created within the task-specific orchestration creator application.

As illustrated in a symbolic block diagram in FIG. 11A, the orchestration 1150 (e.g., task-specific agent) may be instantiated in accordance with the user providing an input directed to the respective user interface element 1111B for interacting with an orchestration that uses data provided by the user (e.g., a medical document, live collection data). In some embodiments, an orchestration (agent) is instantiated based on a time (e.g., at a certain date and/or time), based on an event (e.g., in response to a triggering event), and/or based on a user action. In some embodiments, the orchestration 1150 includes one or more agent-level configurations 1152 (e.g., agent attributes and/or agent settings) and one or more block-level 1154 configurations (e.g., node-level attributes and/or model settings). As shown in FIG. 11A, when the orchestration is instantiated based on the user's input, user-specific data 1128 is provided to the orchestration 1150. In some embodiments, based on the orchestration 1150 being instantiated by the user input directed to the user interface element 1111B, a respective machine-learning model of the task-specific agent is trained (e.g., automatically, without further input provided by the user) on clinician-specific patient data (e.g., precision medicine) based on data associated with the respective user accessing the agent-builder (orchestration) application 1100.

FIG. 11B illustrates an example task-specific orchestration for cell annotation in accordance with some embodiments. FIG. 11B illustrates an example of a user interface 1160 (e.g., a workflow editor) of the agent-builder application 1100 that includes a workflow representation 1161 of an orchestration 1150. The workflow representation 1161 shown in FIG. 11B may be presented based on a user selecting a workflow view for a particular agent.

As depicted by FIG. 11B, the workflow representation 1161 is constructed to represent a node architecture 254 of the orchestration 1150, which may be based on the agent-level configurations 1152 and the block-level configurations 1154. In some embodiments, each workflow representation 1161 configurable by the agent-builder application 1100 includes respective blocks 1162A and 1162B representing an input and an output of the orchestration 1150. For example, the input represented by the block 1162A may include textual content of a prompt, and/or an embedding generated based on the textual content of the prompt. The workflow representation 1161 may also include one or more blocks representing machine-learning models (e.g., a block 1170 representing a large-language model). In some embodiments, the workflow representation 1161 is an interactive representation (e.g., a drag-and-drop representation) in which a user may select an output and then select an input to couple the input to the output (or vice versa). In accordance with some embodiments, each input and output has a corresponding data type (e.g., indicated by a color and/or label). In some embodiments, the system provides suggested building blocks (e.g., agent modules, models, tools, and/or other types of building blocks) based on user prompts. In some embodiments, the system provides a list of available building blocks, and the user may drag and drop the building blocks into the workflow representation to add them to the agent module.

The agent building blocks may include data building blocks, operator building blocks, and/or tool building blocks. Non-limiting examples of data building blocks include an agent listing block (e.g., obtains a listing of available agents), an input block (e.g., accepts a value from a user), a message block (e.g., returns a recent message (and optionally associated metadata) from a conversation), an output block (e.g., returns a response such as a message or document), a history block (e.g., returns a message history), a retrieval bock (e.g., retrieves data, such as documents, from a database or collection), and a semantic block (e.g., identifies semantically similar documents and/or text). Non-limiting examples of operator blocks include a storage block (e.g., configured to store bits of data and/or set common data values with various types), an array block (e.g., configured to transform (e.g., combine) inputs into arrays), a map block (e.g., configured to execute a sub-assembly for inputs in an array and return an array of results), a JSON block (e.g., configured to convert input text to an object via JSON parsing, and optionally validate against a provided schema), an XML block (configured to convert input text to an object via XML parsing, and optionally validate), a status block (e.g., configured provide information about execution status), a template block (e.g., configured to output text in accordance with a given template), and a tool block (e.g., configured to wrap an assembly consumable by another block). Non-limiting examples of tool blocks include an agent tool block (e.g., configured to interface with an agent module), a similarity block (e.g., configured to provide a similarity score for documents), a web block (e.g., configured to operate as an HTTP interface), and a model-tool interface block (e.g., configured to interface between a model and a tool (e.g., ask a model to use a tool)). The workflow representation 1161 in FIG. 11B corresponds to an example summary agent configured to label genetic data (e.g., gene clusters within genetic data).

FIGS. 11C-11D illustrate an example cell annotation in accordance with some embodiments. FIG. 11C illustrates an example gene cluster image in which different gene clusters are denoted, but not labeled. FIG. 11D illustrates the same gene cluster image as FIG. 11C, but the gene clusters are labeled in FIG. 11D. In some embodiments, the gene cluster image in FIG. 11C is input into a summary agent (e.g., the summary agent corresponding to workflow representation 1161) to obtain labeled gene cluster data. In some embodiments, one or more embeddings (e.g., vectors) are generated from the labeled gene cluster data (and provided to one or more ML models).

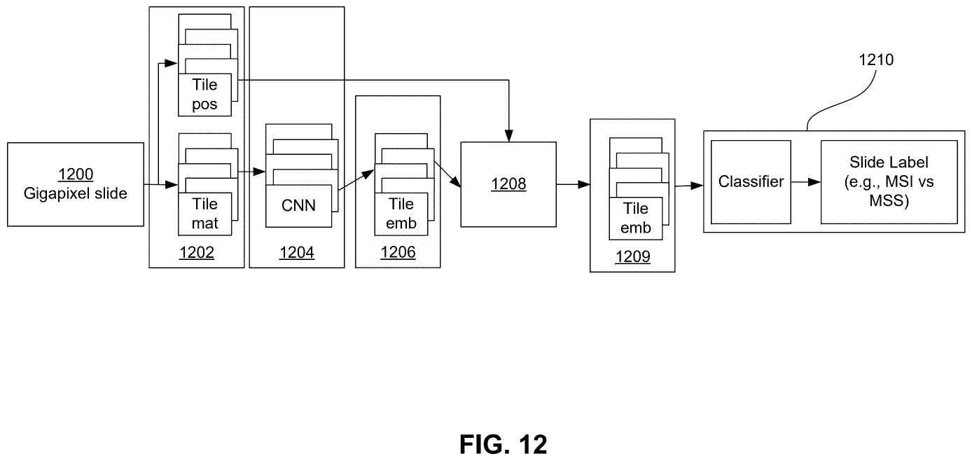

FIG. 12 illustrates an example architecture for slide summarization in accordance with some embodiments. In the example of FIG. 12, a gigapixel image 1200 of a slide (e.g., a slide containing a number of cells) is obtained. In some embodiments, the image 1200 is an image of an H&E slide. In accordance with some embodiments, the image 1200 is partitioned 1202 into a number of tiles and a tile position (e.g., a respective position along an x-axis and y-axis) is maintained for each tile (e.g., as metadata associated with the corresponding tile). In the example of FIG. 12, the individual tiles are provided to an agent module 1204 (e.g., an instance of an agent module 226 or 316) and the agent module 1204 generates a set of one or more tile embeddings 1206 for each tile (e.g., embeddings based on the content of each tile). In accordance with some embodiments, the agent module 1204 utilizes a set of convolutional neural networks (CNNs) to generate the tile embeddings. In accordance with some embodiments, the tile embeddings 1206 are provided to an agent module 1208 (e.g., that includes a self-attention neural network) with the tile positions to generate updated tile embeddings 1209 (e.g., embeddings based on the content and position of each tile). In some embodiments, the tile positions are used to interpret the content of the current tile based on the content of surrounding tiles. In accordance with some embodiments, the updated tile embeddings 1209 are provided to an agent module 1210 that is configured to label the slide 1200 based on the updated tile embeddings (e.g., using a classifier component). For example, the classifier may be configured to label a slide as containing a microsatellite instability (MSI) or a microsatellite stability (MSS). In some embodiments, undeciphered embeddings from each slide are used as vectors of weights. In some embodiments, model outputs from analysis of the slide (e.g., from an image-to-text model) are used as a textual summary of the identified cells, tissues, and/or biomarkers. In some embodiments, embeddings are derived and stored a tile level (e.g., for responding to subsequent queries), which reduces the amount of tile data stored a slide image (as compared to storing the entire slide image 1200).

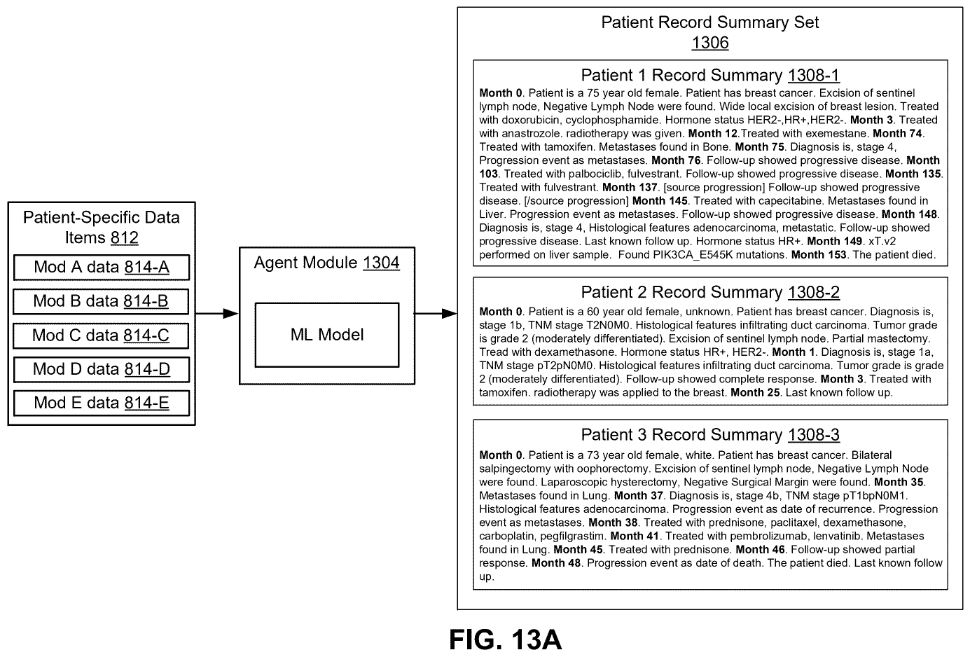

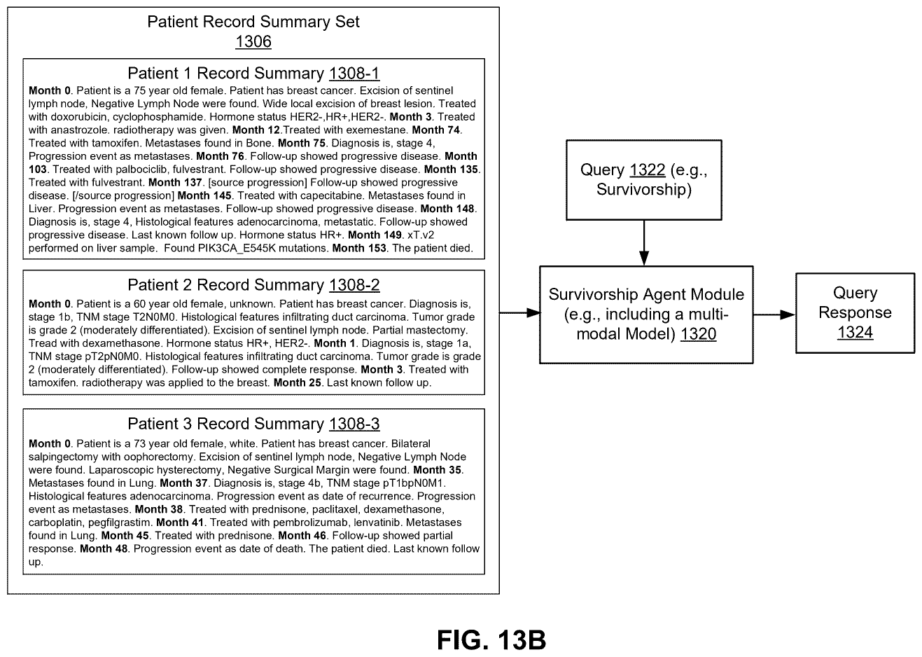

FIGS. 13A-13B illustrate an example architecture and procedure for generating inferences on survivorship in accordance with some embodiments. FIG. 13A shows the patient-specific data items 812 (discussed above with response to FIGS. 8A-8B) being input into an agent module 1304 (e.g., an instance of an agent module 226 or 316). As discussed previously the patient-specific data items 812 may include multiple data modalities (e.g., modality 814-A through 814-E). The agent module 1304 includes an ML model and is configured to generate a patient record summary set 1306 from the patient-specific data items 812. FIG. 13A shows an example in which the patient record summary set 1306 includes a textual record summary 1308 for each patient. Each record summary 1308 in FIG. 13A includes key events and details from the patient record organized chronologically by month. In accordance with some embodiments, timing of the key events and details is also included in the record summary 1308. In some embodiments, other organizational schemes are used. In some embodiments, each record summary 1308 is used to generate a corresponding patient embedding (e.g., a multi-dimensional vector). Generating embeddings from summaries in this manner can be used to conserve generalizable representations of the underlying data.

FIG. 13B shows an example of using the patient record summary set 1306 (e.g., generated as illustrated in FIG. 13A) to respond to a query 1322 about survivorship. In the example of FIG. 13B, the patient record summary set 1306 and the query 1322 are provided to a survivorship agent module 1320 (which may include a multi-modal model). In accordance with some embodiments, the survivorship agent module 1320 is configured to generate a query response 1324 (e.g., a survivorship estimate) based on the query 1322 and the patient record summary set 1306. In some embodiments, embeddings of the patient record summary set 1306 are provided to the survivorship agent module 1320. In some embodiments, the survivorship agent module 1320 is configured to generate embeddings of the patient record summary set 1306. In some embodiments the survivorship agent module 1320 is configured to provide other types of outputs related to survivorship, such as predicting drug responses and/or providing (e.g., ranking) treatment options (e.g., therapy options).

Various example embodiments and aspects of the disclosure are described below for convenience. These are provided as examples, and do not limit the subject technology. Some of the examples described below are illustrated with respect to the figures disclosed herein simply for illustration purposes without limiting the scope of the subject technology.

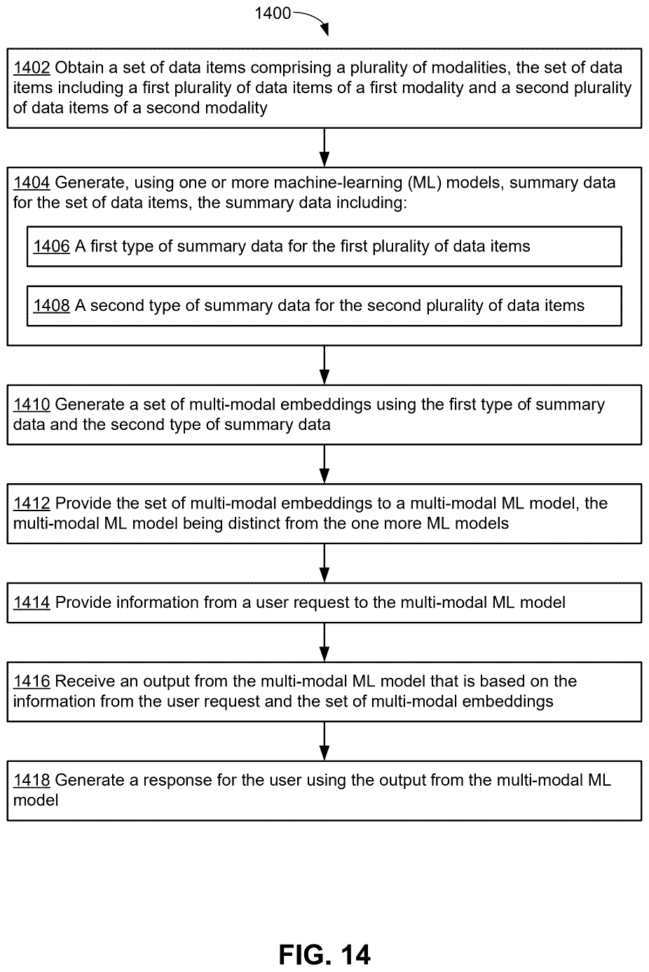

FIG. 14 is a flow diagram illustrating an example method 1400 of generating inferences from multi-modal data in accordance with some embodiments. The method 1400 is performed at a computing system (e.g., a client device, server system, and/or service platform) having one or more processors (e.g., the CPUs 202 and/or 302) and memory (e.g., the memory 218 and/or 310). In some embodiments, the memory stores one or more programs configured for execution by the one or more processors. At least some of the operations shown in FIG. 14 correspond to instructions stored in a computer memory or a computer-readable storage medium. In some embodiments, the computing system is the platform 100, the client device(s) 102, and/or the server system 106. In some embodiments, the computing system comprises a set of agents, agent modules, orchestrations, and/or ML models.

(A1) In one aspect, some embodiments include the method 1400 for generating inferences from multi-modal data performed at a computing system. The computing system obtains (1402) a set of data items (e.g., the patient-specific data items 812) comprising a plurality of modalities, the set of data items including a first plurality of data items of a first modality (e.g., the modality A data 814-A) and a second plurality of data items of a second modality (e.g., the modality D data 814-D). The computing system generates (1404), using one or more machine-learning (ML) models (e.g., one or more of the ML model(s) 228 and/or the ML models 318), summary data for the set of data items, the summary data including: a first type of summary data (1406) for the first plurality of data items (e.g., the characterized molecular data 612), and a second type of summary data (1408) for the second plurality of data items (e.g., the text summary 614). The computing system generates (1410) a set of multi-modal embeddings (e.g., the aggregated embeddings 630) using the first type of summary data and the second type of summary data. The computing system provides (1412) the set of multi-modal embeddings to a multi-modal ML model (e.g., the multi-modal model 818), the multi-modal ML model being distinct from the one more ML models. The computing system provides (1414) information from a user request (e.g., the query data 806) to the multi-modal ML model. The computing system receives (1416) an output from the multi-modal ML model (e.g., the output 634) that is based on the information from the user request and the set of multi-modal embeddings. The computing system generates (1418) a response for the user using the output from the multi-modal ML model (e.g., the output response 822). For example, a user may send a query relevant to the first plurality of data items, the second plurality of data items, or both. In some embodiments, a prompt is provided to the multi-modal ML model, the prompt including the information from the user query and additional information (e.g., response instructions, context information, and the like). In some embodiments, the response relates to a cohort referenced in the user prompt. As an example, the response may correspond to a care plan for a patient, identification of a medical condition of a patient, care instructions for a patient, and/or an assessment of a patient. In some embodiments, the response to the user is a natural language output. In some embodiments, the natural language output summarizes the output from the multi-modal ML model. In some embodiments, the natural language output incorporates output from two or more ML models (e.g., information from the user request is provided to two or more models and the outputs are compared/combined to generate the response).

In some embodiments, receiving information corresponding to a user request comprises receiving content of a user query, context for the user query, and/or metadata associated with the user query. In some embodiments, the user request is received via a user interface (e.g., a user interface corresponding to a digital assistant agent). In some embodiments, the user request comprises a natural language input.

In some embodiments, the set of data items are obtained from a medical database. In some embodiments, the set of data items are obtained from two or more medical databases. In some embodiments, the first plurality of data items and the second plurality of data items are composed of (e.g., consist of) de-identified data. For example, all patient data in the first plurality of data items and the second plurality of data items is de-identified. As an example, the first plurality of data items and the second plurality of data items do not contain any PII or PHI. In some embodiments, the set of data items comprise medical data. For example, the medical data may include patient records, treatment options, therapy instructions, and/or clinical publications. In some embodiments, the first plurality of data items and the second plurality of data items are obtained from a same database. In some embodiments, the first plurality of data items and the second plurality of data items correspond to a set of subjects. In some embodiments, the first plurality of data items corresponds to a first set of patients and the second plurality of data items corresponds to a second set of patients. In some embodiments, the first set of patients at least partially overlaps with the second set of patients. In some embodiments, the first set of patients is the same as the second set of patients. In some embodiments, the first plurality of data items and the second plurality of data items correspond to a set of (one or more) cohorts.

In some embodiments, the database is a client (third-party) database. In some embodiments, the first plurality of data items and the second plurality of data items are obtained from different databases. The database(s) may comprise a medical database, a patient database, and/or a treatment database. In some embodiments, at least one of the first plurality of data items and the second plurality of data items comprises medical records. The medical records may comprise EHRs and/or EMRs. For example, the medical records may include demographic information for a set of patients, care plan details for a set of patients, therapies administered to a set of patients, care instructions for a set of patients, and/or clinical publications.

In some embodiments, information from the user request (e.g., the same information, or different information from the user request) is provided to a second ML model and the response for the user is generated based on outputs from the multi-modal ML model and the second ML model. In some embodiments, generating the output comprises identifying agreement between the respective inferences from two or more ML models. For example, the user query may relate to identifying a care plan for a patient and the output includes care plan details that are indicated by two or more of the ML models. In some embodiments, the response for the user indicates which information is coming from which ML model. For example, information from each output may be used in the output along with a note regarding which model provided the particular information. In some embodiments, the response is generated based on an output having the highest associated confidence value. In some embodiments, outputs with confidence values below a threshold value are not used to generate the response (e.g., are discarded). In some embodiments, only the output with the highest confidence value is used. In some embodiments, the top K outputs based on confidence values are used.

In some embodiments, the one or more ML models comprise a large language model (LLM). In some embodiments, the one or more ML models comprise one or more transformer-based models. In some embodiments, the one or more ML models comprise a model configured to capture long range dependencies.

Although FIG. 14 illustrates a number of logical stages in a particular order, stages which are not order dependent may be reordered and other stages may be combined or broken out. Some reordering or other groupings not specifically mentioned will be apparent to those of ordinary skill in the art, so the ordering and groupings presented herein are not exhaustive. For example, the set of multi-modal embeddings may be provided to the multi-modal ML model before, concurrent with, or after the information from the user request. Moreover, it should be recognized that various stages could be implemented in hardware, firmware, software, or any combination thereof.

(A2) In some embodiments of A1, the one or more ML models are components of a set of task-specific orchestrations (e.g., agents trained to perform specific tasks). In some embodiments, each modality is summarized by a different task-specific orchestration. In some embodiments, each task-specific orchestration comprises one or more ML models.

(A3) In some embodiments of A1 or A2, the first modality or the second modality comprises text, and the first type of summary data or the second type of summary data comprises a summarization of the text. For example, each paragraph, section, page, document, note, and/or chapter may be summarized. In some embodiments, the first modality comprises structured text, unstructured text, tabular data, data visualizations, images, audio, video, biological sequence data, natural language data, or source code. In some embodiments,

(A4) In some embodiments of any of A1-A3, the first modality or the second modality comprises images, and the first type of summary data or the second type of summary data comprises edited images. In some embodiments, the edited images are cropped, annotated, sharpened, and/or otherwise edited. In some embodiments, the second modality comprises structured text, unstructured text, tabular data, data visualizations, images, audio, video, biological sequence data, natural language data, or source code.

(A5) In some embodiments of any of A1-A4, the first modality or the second modality comprises a pathology slide image (e.g., the slide image 1200), and the first type of summary data or the second type of summary data comprises an annotated version of the pathology slide image.

(A6) In some embodiments of any of A1-A5: (i) the set of data items correspond to an electronic health record for a cancer subject; (ii) the summary data comprises a chronological account of medical events involving the cancer subject; (iii) the user request comprises a request to calculate overall survivorship (OS) for the cancer subject; and (iv) the output from the multi-modal ML model indicates the OS for the cancer subject. The medical events may include treatments applied, medications taken, dosage information, tests applied, test results, disease progression, and/or diagnoses. The set of data items may include patient demographics, cancer stage data, cancer grade data, histology, procedures (and corresponding outcomes) data, medications data, radiotherapy data, molecular data, mutations data, hormone data, metastases data, progression (events) data, and/or oncology data. In some embodiments, the set of data items comprise imaging data (e.g., ECG, echocardiogram, etc.), molecular data, pathology data, radiology data, text data, and the like. In some embodiments, the user request comprises a request to identify and/or characterize a disease state based on the summary data (e.g., cardiovascular diseases, cancers, endocrine diseases, or other disease states), and the output from the multi-modal ML model indicates the disease state (e.g., identifies the disease state, characterizes the disease state, and/or summarizes the disease state).

(A7) In some embodiments of any of A1-A6, the method further includes: (i) obtaining a third plurality of data items of a third modality (e.g., the modality C data 814-C); and (ii) generating a third type of summary data (e.g., the labeled image data 606) for the third plurality of data items, where the multi-modal embeddings are generated using the first type of summary data, the second type of summary data, and the third type of summary data. In some embodiments, the set of multi-modal embeddings are generated using data of four or more data modalities.

(A8) In some embodiments of any of A1-A7, the method further comprises: (i) generating a first set of embeddings (e.g., the molecular data embeddings 622) from the first type of summary data; and (ii) generating a second set of embeddings (e.g., the textual data embeddings 624) from the second type of summary data, where the set of multi-modal embeddings are generated by aggregating the first and second sets of embeddings.

(A9) In some embodiments of any of A1-A8, the multi-modal ML model is a component of an orchestration (e.g., a multi-modal agent). In some embodiments, the orchestration is a task-specific orchestration. For example, the task-specific orchestration is a multi-modal agent. In some embodiments, the task-specific orchestration is selected based on the type of output to be provided. For example, the task-specific orchestration is selected from a plurality of task-specific orchestration based on the type of response to be provided.

For example, each task-specific orchestration in the plurality of task-specific orchestration may be associated with one or more output types. For example, a query regarding a regimen recommendation may use a first subset of (one or more) ML models, and a query regarding a cohort may use a second subset of (one or more) ML models. In some embodiments, a same ML model is used for multiple response types. In some embodiments, each ML model is used for a respective response type. In some embodiments, the task-specific orchestration is selected based on a type of data being requested by the user request. As an example, a query regarding a regimen recommendation may use a first subset of (one or more) ML models, and a query regarding a cohort may use a second subset of (one or more) ML models.

In some embodiments, the one or more machine-learning models correspond to a set of one or more task-specific orchestrations and the multi-modal machine-learning model corresponds to a different task-specific orchestration. In some embodiments, the multi-modal ML model is selected in accordance with a determination that the user request relates to multi-modal data. In some embodiments, in accordance with a determination that a user request relates to single modality data, a different ML model is selected. For example, in accordance with a determination that the set of data items consist of a single modality of data, a different ML model is selected to generate a response. In some embodiments, the multi-modal ML model is trained using multi-modal data.

In some embodiments, the multi-modal ML model is trained to assign little or no weight to default embeddings (as compared to other embeddings). In some embodiments, the multi-modal ML model is trained using data with default embedding (e.g., so that the multi-modal ML model is trained to give little weight to the default embeddings). In some embodiments, generating an inference (or other output) comprises applying a negligible weight to the default embedding. For example, the default embedding is given a smallest weight, a weight that is an order of magnitude less than weights of other embeddings, and/or a weight of zero.

In some embodiments, the multi-modal ML model is configured to determine which embeddings of the set of multi-modal embeddings are most relevant (closest in a vector space) to the user request.

(A10) In some embodiments of any of A1-A9, the method further includes: (i) selecting, from the one or more ML models, a first ML model for generating the first type of summary data, where the first ML model is selected based on the first modality; and (ii) selecting, from the one or more ML models, a second ML model for generating the second type of summary data, where the second ML model is selected based on the second modality. In some embodiments, each ML model is designated for use with one or more respective data modalities. In some embodiments, the first ML model is trained using the first modality of data, and the second ML model is trained using the second modality of data.

(A11) In some embodiments of any of A1-A10, generating the set of multi-modal embeddings comprises incorporating default data (e.g., the default embeddings 708) into the set of multi-modal embeddings in accordance with a determination that the set of data items is missing data. In some embodiments, a method of generating an inference, includes: (i) receiving an identification of a subject; (ii) based on the identification of the subject, obtaining a set of data items relating to the subject; (iii) generating a set of embeddings from the set of data items, including, for each modality of a plurality of modalities: (iv) in accordance with a determination that the set of data items includes a subset of data items having the modality, generating one or more embeddings for the subset of data items; and (v) in accordance with a determination that the set of data items does not include any data items having the modality, using a default embedding for the modality; and (vi) generating, via a multi-modal ML model, the inference based on the set of embeddings. In some embodiments, the identification of the subject comprises a patient identifier. In some embodiments, the set of data items comprise de-identified patient data. In some embodiments, the default embedding corresponds to a type of data that is not included in a medical record of the subject. For example, a first default embedding may be used if the patient record does not include x-ray data. A second default embedding may be used if the patient record does not include biological sequencing data. A third default embedding may be used if the patient record does not include clinical notes.

(A12) In some embodiments of any of A1-A11, the plurality of modalities comprises one or more of: a structured text modality, an unstructured text modality, a tabular data modality, a data visualizations modality, an image modality, an audio modality, a video modality, a biological sequence modality, a natural language modality, and a source code modality. In some embodiments, the plurality of modalities includes a first modality for a first type of images (e.g., x-ray images) and a second modality for a second type of images (e.g., ultrasound images). In some embodiments, the plurality of modalities comprises three or more modalities. In some embodiments, the plurality of modalities correspond to different parts of a patient record.

(A13) In some embodiments of any of A1-A12, the set of multi-modal embeddings are generated using a set of ML models. In some embodiments, the set of embedding are generated using a first task-specific orchestration (e.g., that includes the first ML model). In some embodiments, the first ML model is distinct from the multi-modal ML model. In some embodiments, the set of ML models is distinct from the one or more ML models. In some embodiments, the set of ML models includes a model for each modality in the plurality of modalities. In some embodiments, the set of ML models includes an aggregation model or tool for aggregating modality-specific embeddings to generate the set of multi-modal embeddings. In some embodiments, the set of multi-modal embeddings correspond to a single subject. In some embodiments, the set of multi-modal embeddings correspond to a cohort of subjects. In some embodiments, each subject has a corresponding data set related to the subject's medical record. As an example, a different set of data may be missing from each subject's corresponding medical record.

(A14) In some embodiments of any of A1-A13, the response for the user comprises an indication of which data modalities from the plurality of modalities were used to generate the response (e.g., the indication 824). For example, the response identifies subjects that are smokers and indicates whether this conclusion is based on text data, image data, and/or audio data. In some embodiments, the modalities that were used to generate the response are identified based on which agents/models were used and/or provided an output (e.g., an output having a confidence level that exceeds a threshold value).

In some embodiments, a method of responding to user queries includes: (i) receiving information corresponding to a user query from a user; (ii) determining which modalities of data of a set of enumerated modalities relate to the user query; (iii) sending a request to one or more machine-learning (ML) models to generate respective responses to the user query, including: (iv) in accordance with a determination that multiple modalities of data relate to the user query, sending a request to a multi-modal ML model to generate a response to the user query; and (v) in accordance with a determination that an enumerated modality of data of the set of enumerated modalities relates to the user query, sending a request to a second ML model to generate a response to the user query, wherein the second ML model is trained based on data of the enumerated modality; (vi) receiving the respective responses from the one or more ML models; and (vii) generating an output for the user based on the respective responses from the one or more ML models. In some embodiments, the user query is provided to the multi-modal ML model and the modalities of the data are determined based on a response from the multi-modal ML model.

In some embodiments, the multi-modal ML model provides an inference along with an indication of how relevant each modality was to the inference. In some embodiments, the modalities of data are determined based on the response from the multi-modal ML model. For example, the user query is provided to the multi-modal ML model and the multi-modal ML model provides a response that indicates which modalities of data relate to the user query. In some embodiments, the response from the multi-modal ML model indicates a relative contribution from each data modality of the plurality of data modalities. In some embodiments, a modality is determined to relate to the user query when the relative contribution exceeds a threshold (e.g., a threshold of 0.1, 0.2, or 0.3). In some embodiments, the top K enumerated modalities are determined to relate to the user query, where K is a positive integer.

In some embodiments, the modalities of data used in the response are determined based on the user request. For example, the user request requests a response with a particular modality of data and/or requests information about a particular modality of data. In some embodiments, the modalities of data are explicitly identified in the user query. In some embodiments, the modalities of data are not explicitly identified in the user query. In some embodiments, the modalities of data are identified based on analysis of the user query (and optionally contextual information for the user query).

In some embodiments, the modalities of data used in the response are determined based on a set of embeddings determined to be relevant to the user query. For example, the user query is input into an ML model and the set of embeddings are identified as being the most relevant in a vector space. In this example, the set of embeddings are analyzed to determine which modalities of data were used to generate the embeddings.

(A15) In some embodiments of any of A1-A14, the response for the user includes an indication of what source data was used to generate the output from the multi-modal model (e.g., as illustrated by the source material column 1036 in FIG. 10I). In some embodiments, the response for the user includes an indication of which data items (and/or which portions of the data items) from the set of data items were used to generate the output from the multi-modal ML model. For example, a document identifier or a snippet of content from the document (e.g., the content corresponding to an embedding determined to be relevant) is provided with (or as part of) the response. In some embodiments, generating the response for the user comprises providing a short answer, a long answer, and an indication of relevant source documents. For example, the short answer may be a yes or no statement and the long answer may include logic/reasoning for the short answer. For example, the user may show or hide the individual components within the user interface.

(A16) In some embodiments of any of A1-A15, the method further includes: (i) determining which modalities of the plurality of modalities were used to generate the output from the multi-modal ML model (e.g., using the agent module 810); (ii) based on the determined modalities, sending a request to a second ML model (e.g., the single-modal model 819) to generate an output responsive to the user request, where the second ML model is different than the one or more ML models and the multi-modal ML model; and (iii) receiving, from the second ML model, an additional output responsive to the user request, where the response for the user is generated based on the additional output. In some embodiments, the response is generated based on agreement between the outputs from the models. In some embodiments, the response is generated by incorporating information from the additional output, but not the (initial) output from the multi-modal ML model.

(A17) In some embodiments of any of A1-A16, the method further includes: (i) identifying an output type (e.g., one of the sets of criteria 906) for the output from the multi-modal ML model; (ii) identifying one or more criteria for the output based on the identified output type; and (iii) determining whether the output from the multi-modal ML model meets the one or more criteria, where the response for the user is generated in accordance with a determination that the output from the multi-modal ML model meets the one or more criteria. The types of outputs may include a care plan output type, a therapy output type, a medical assessment output type, and a patient output type. The output types may depend on a modality of data included in the user query and/or to be assessed to generate the response. In some embodiments, the output type correlates with a task type that is indicated in the output. In some embodiments, in accordance with a determination that the output from the multi-modal ML model does not meet the one or more criteria, providing a response indicating that the output from the multi-modal ML model is invalid. For example, the response may indicate that additional information is needed and/or the initial user request may have incorrect information. In some embodiments, in accordance with a determination that the output from the multi-modal ML model does not meet the one or more criteria, a second request is provided to the multi-modal ML model to generate a second response to the user query. For example, the second request may be rephrased, may include different information from the user query, may include information about the one or more criteria, and/or may include information about why the first response did not meet the one or more criteria. In some embodiments, the second request includes information regarding the response from the multi-modal ML model not meeting the one or more criteria. For example, the second request may include an indication of the one or more criteria and/or an indication of why the first output did not meet the criteria. In some embodiments, in accordance with a determination that the response from the multi-modal ML model does not meet the one or more criteria, a second request is provided to a second ML model to generate a second response to the user query. In some embodiments, the second ML model is trained on different data than the multi-modal ML model. In some embodiments, the second ML model has different parameters (and/or hyperparameters) than the multi-modal ML model. In some embodiments, the multi-modal and second ML models are a same type of model. In some embodiments, the multi-modal and second ML models are different types of models.

In some embodiments, the one or more criteria are predefined based on one or more of medical information, treatment information, logical fallacies, one or more policy rules, and one or more regulations. For example, the one or more criteria may be setup to ensure that an output is logically sound, follows relevant medical guidelines, and complies with any company policies. In some embodiments, at least a subset of the one or more policies rules and the one or more regulations are specific to the type of response to be provided. In some embodiments, separate policy rules and/or regulations are provided for each type of response.

In some embodiments, the type of output to be provided is identified based on a type of question in the user query (e.g., based on information from the prompt 808). In some embodiments, the type of output to be provided is identified based on context for the user query (e.g., information about the user, past interactions with the user, a state of an application in which the user query is received, and the like). In some embodiments, the type of response to be provided is identified based on a prompt generated for the user query.

(B1) In another aspect, some embodiments include a method of generating query responses performed at a computing system. The method includes: (i) receiving, via a user interface element of a user interface, a request to import a source dataset corresponding to a plurality of subjects (e.g., as illustrated in FIG. 10D); (ii) in response to the request, importing the source dataset; (iii) generating a plurality of embeddings from the source dataset; (iv) receiving, from a user via the user interface, a query for information from the source dataset; (v) generating an output for the user query using a task-specific orchestration and the plurality of embeddings; and (vi) presenting the output to the user via the user interface, the output including a respective response for each of the plurality of subjects. In some embodiments, the output includes a short answer (e.g., a final answer), a long answer, and an indication of the corresponding source document for each subject. In some embodiments, the source dataset is obtained from a third-party database, a client database, or other external database. In some embodiments, the user interface includes a row for each subject and each row includes a column for the query, a column for the respective responses, and optionally a column indicating analysis used to determine the respective responses.

(B2) In some embodiments of B1, the method further includes: (i) obtaining information from a second dataset (e.g., from the database(s) 350); and (ii) combining the information from the second dataset with information from the source dataset (e.g., as illustrated in FIG. 10J), where the output is generated based on the combined information from the source dataset and the second dataset. In some embodiments, the second dataset is obtained from an internal database. In some embodiments, the information from the second dataset comprises a set of embeddings. In some embodiments, the information from the second dataset comprises structured data. In some embodiments, the information from the second dataset comprises one or more modalities of data not included in the source dataset.

(B3) In some embodiments of B1 or B2, the plurality of embeddings comprise a set of word embeddings. As discussed previously, word embeddings capture semantic relationships between words (which allows a model to understand and represent words in a vector space).

(B4) In some embodiments of any of B1-B3, the output includes an indication of the analysis that was used to determine each respective response (e.g., in a long answer field).

(B5) In some embodiments of any of B1-B4, the output includes an indication of a portion of the source dataset that was used to determine each respective response (e.g., the source material shown in FIG. 10I).

(B6) In some embodiments of any of B1-B5, the source dataset includes a set of patients, and the method further includes de-identifying the set of patients before generating the plurality of embeddings from the source dataset.

(B7) In some embodiments of any of B1-B6, the method further includes, in accordance with receiving the query for information from the source dataset, adding a respective row to the user interface for each respective subject of the plurality of subjects of the source dataset (e.g., as illustrated in FIG. 10F).

(B8) In some embodiments of any of B1-B7, the task-specific orchestration is selected based on content of the query. In some embodiments, the task-specific orchestration is selected based on a query type of the query. In some embodiments, the task-specific orchestration is selected based on a concept embodied in the query.

(B9) In some embodiments of any of B1-B8: (i) the source dataset comprises unstructured data; and (ii) importing source dataset comprises converting the unstructured data to structured data. In some embodiments, the source dataset comprises structured and unstructured data. In some embodiments, the source dataset comprises a plurality of data modalities.

(B10) In some embodiments of any of B1-B9, the method further includes, in response to the query for information from the source dataset, applying the query and respective data from the source dataset to a second task-specific orchestration, distinct from the task-specific orchestration, to validate that there is sufficient data for the task-specific orchestration to generate an output for the user query.

(B11) In some embodiments of B10: (i) the task-specific orchestration comprises a RAG architecture; and (ii) the method further includes, in accordance with determining that there is not sufficient information to resolve the user query, providing the user query to a third task-specific orchestration that does not comprise a RAG architecture. In some embodiments, in accordance with a determination that there is sufficient data for the task-specific orchestration to generate an output for the user query, generating the response to the user query using the task-specific orchestration. In some embodiments, the output from the third task-specific orchestration is combined with the output from the RAG architecture.

(C1) In another aspect, some embodiments include a method of labeling genetic data performed at a computing system. The method includes: (i) obtaining a set of genetic data (e.g., the genetic data illustrated in FIG. 11C) that includes information about a plurality of gene clusters and a plurality of cluster marker genes; (ii) providing the genetic data to an agent (e.g., comprising a ML model) with a request to annotate the plurality of gene clusters using the plurality of cluster marker genes; (iii) receiving a response from the agent; and (iv) labeling the plurality of gene clusters according to the response (e.g., as illustrated in FIG. 11D). In some embodiments, the labeled gene clusters are tokenized (e.g., gene and mutations are tokenized as embeddings for a model).

(C2) In some embodiments of C1, the method further includes: (i) in response to providing the genetic data, receiving a first response from the ML model; and (ii) in response to the first response, sending a second request to the ML model, the second request instructing the ML model to be more specific, where the response from the ML model is responsive to the second request.

(C3) In some embodiments of C1 or C2, the plurality of cluster marker genes are obtained via a first type of data analysis of the plurality of gene clusters (e.g., a cluster analysis).

(C4) In some embodiments of any of C1-C3, the set of genetic data is obtained via a single-cell analysis pipeline. In some embodiments, the single-cell analysis pipeline includes at least one of raw sequencing output analysis (e.g., using raw base call (BCL) files), conversion of the raw sequencing output to text (e.g., converting BCL files to FASTQ files), generating a count matrix (e.g., indicating a count of cells for each gene), and generating a quality control (QC) information.

(C5) In some embodiments of any of C1-C4, the set of genetic data is obtained from a set of tissue samples. For example, the set of tissue samples are dissociated, sorted, and/or prepared in a modeling lab. Then the set of tissue samples may undergo cell partitioning and sequencing.

(C6) In some embodiments of any of C1-C5, the ML model is a component of a task-specific orchestration (e.g., a component of a genetic-analysis agent).

(C7) In some embodiments of any of C1-C6, the ML model comprises a large language model. In some embodiments, the ML model comprises a transformer model.

(C8) In some embodiments of any of C1-C7, the ML model is trained on an RNA data modality. In some embodiments, the ML model is fine-tuned using the RNA data modality. In some embodiments, the ML model is prompted to consider RNA data.

(C9) In some embodiments of any of C1-C8, the information about the plurality of gene clusters comprises data of an image modality.

(C10) In some embodiments of any of C1-C9, the method further includes generating a set of embeddings for the genetic data based on the labeled plurality of gene clusters.

(D1) In another aspect, some embodiments include a method of labeling genetic data performed at a computing system. The method includes: (i) obtaining a pathology slide image (e.g., the slide image 1200); (ii) partitioning the pathology slide image into a plurality of tiles; (iii) storing a plurality of tile positions corresponding to the plurality of tiles; (iv) obtaining a plurality of tile embeddings (e.g., the tile embeddings 1206) by generating, for each title of the plurality of tiles, a tile embedding; (v) inputting the plurality of tile embeddings and the plurality of tile positions to a self-attention ML model; (vi) receiving an output from the self-attention ML model; and (vii) labeling the pathology slide image according to the output. For example, a slide may be partitioned into 10 tiles, 100 tiles, or 1000 tiles. In some embodiments, the self-attention ML model is trained via a multiple instance learning (MIL) training technique.

(D2) In some embodiments of D1, the ML model comprises a self-attention neural network and a classifier.

(D3) In some embodiments of D2, the self-attention neural network comprises a plurality of self-attention layers. In some embodiments, each self-attention layer of the plurality of self-attention layers comprises a plurality of self-attention heads and a multi-layer perceptron. In some embodiments, each self-attention head of the plurality of self-attention heads is trained to learn tile interpretations based on tile contents and the tile position information. In some embodiments, each self-attention head of the plurality of self-attention heads is configured for content self-attention or position self-attention. In some embodiments, each self-attention head of the plurality of self-attention heads comprises a position encoder, a position scorer, a content scorer, and a combiner component for combining the position score and the content score.

(D4) In some embodiments of any of D1-D3, labeling the pathology slide image comprises assigning a label of MSS or MSI.

(D5) In some embodiments of any of D1-D4, each tile of the plurality of tiles comprises a tile matrix of pixel values.

(D6) In some embodiments of any of D1-D5, the plurality of tile embeddings are generated using a second ML model. For example, the second ML model may comprise a neural network, such as a convolutional neural network.

(D7) In some embodiments of any of D1-D6, each tile position of the plurality of tile positions indicates a two-dimensional position of the corresponding tile. For example, the two-dimensional position may be an x, y coordinate of the top right corner of the tile. As another example, the two-dimensional position may be an x, y coordinate of the center of the tile.

(D8) In some embodiments of any of D1-D7, the plurality of tile positions comprise embeddings of tile positions. For example, the embeddings of tile positions may be generated using sine and/or cosine waves of different wavelengths. In some embodiments, each embedding of tile positions comprises a matrix of relative distances to other tiles of the plurality of tiles.

(D9) In some embodiments of any of D1-D8, the self-attention ML model is configured to combine a content self-attention and a position self-attention for each tile of the plurality of tiles.

(D10) In some embodiments of any of D1-D9, the method further includes generating an embedding for the labeled pathology slide image.

(E1) In another aspect, some embodiments include a method of determining overall survivorship (OS) performed at a computing system. The method includes: (i) obtaining multi-modal data for a subject (e.g., the patient-specific data items 812), the multi-modal data corresponding to a plurality of modalities; (ii) generating a set of textual strings from the multi-modal data (e.g., the patient record summary set 1306); (iii) inputting the set of textual strings to a ML model with a prompt to determine an overall survivorship (OS) for the subject; and (iv) receiving a response from the ML model, the response indicating the OS for the subject.

(E2) In some embodiments of E1, the multi-modal data comprises one or more of: a demographic data modality, a clinical data modality, and a molecular data modality. For example, the multi-modal data may include one or more of patient demographics, cancer stage data, cancer grade data, histology, procedures (and corresponding outcomes) data, medications data, radiotherapy data, molecular data, mutations data, hormone data, metastases data, progression (events) data, and oncology data.

(E3) In some embodiments of E1 or E2, each textual string comprises data from each modality of the plurality of modalities. In some embodiments, each textual string includes curated and native data. In some embodiments, each textual string includes clinical and molecular data.

(E4) In some embodiments of any of E1-E3, the OS is determined from a disease onset.

(E5) In some embodiments of any of E1-E3, the OS is determined from a first metastatic diagnosis (MET).

(E6) In some embodiments of any of E1-E5, the set of textual strings are generated in a physician notes style. For example, the set of textual strings are configured to imitate physician notes of a patient journey. In some embodiments, generating the set of textual strings from the multi-modal data comprises converting the multi-modal data to the set of textual strings. In some embodiments, each textual string is arranged in a chronological order. In some embodiments, each textual string comprises temporal-based text.

(E7) In some embodiments of any of E1-E6, the ML model comprises a large language model. In some embodiments, the ML model comprises a transformer model. In some embodiments, the ML model is a component of a task-specific orchestration.

(E8) In some embodiments of any of E1-E7, the subject is a cancer patient. For example, the subject may be a patient having metastatic breast cancer.

(E9) In some embodiments of any of E1-E8, the set of textual strings is generated from the multi-modal data using a second ML model. In some embodiments, the second ML model comprises a large language model. In some embodiments, the second ML model comprises a transformer model. In some embodiments, the second ML model is a component of a second task-specific orchestration.

(E10) In some embodiments of any of E1-E9, the subject is a member of a cohort of cancer patients.

(E11) In some embodiments of any of E1-E10, the multi-modal data is obtained from a patient database. In some embodiments, the multi-modal data is obtained from a client database, a third-party database, a medical database, or other type of database. In some embodiments, the multi-modal data is obtained from a patient medical record.

In another aspect, some embodiments include a computing system (e.g., a client device 102, a server system 106, and/or the platform 100) including control circuitry and memory coupled to the control circuitry, the memory storing one or more sets of instructions configured to be executed by the control circuitry, the one or more sets of instructions including instructions for performing any of the methods described herein (e.g., the method 1400 as well as A1-A17, B1-B11, C1-C10, D1-D10, and E1-E11 above).

In another aspect, some embodiments include a non-transitory computer-readable storage medium storing one or more sets of instructions for execution by control circuitry of a computing system, the one or more sets of instructions including instructions for performing one or more of the methods described herein (e.g., the method 1400 as well as A1-A17, B1-B11, C1-C10, D1-D10, and E1-E11 above).

Various types of models and algorithms may be used with the agents and components disclosed herein. In some embodiments, a model is a supervised machine learning algorithm. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level classifier).

In some embodiments, a model is, or includes, a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of network nodes organized into multiple layers of network nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm can be a neural network comprising a plurality of hidden layers, e.g., two or more hidden layers. Each layer of the neural network can comprise a number of network nodes (also sometimes referred to as neurons). A network node can receive input that comes either directly from the input data or the output of network nodes in previous layers, and perform a specific operation, e.g., a summation operation. In some embodiments, a connection from an input to a network node is associated with a parameter (e.g., a weight and/or weighting factor). In some embodiments, a network node sums up the products of all pairs of inputs, Xi, and their associated parameters. In some embodiments, the weighted sum is offset with a bias, b. In some embodiments, the output of a network node is gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

As an example, a variety of neural networks may be suitable for use in analyzing an image of an eye of a subject. Examples can include, but are not limited to, feedforward neural networks, radial basis function networks, recurrent neural networks, residual neural networks, convolutional neural networks, residual convolutional neural networks, and the like, or any combination thereof. In some embodiments, a machine-learning model uses a pre-trained and/or transfer-learned ANN or deep learning architecture. Convolutional and/or residual neural networks can be used for analyzing an image of a subject in accordance with the present disclosure. Some embodiments use generative models, such as generative adversarial networks (GANs) and hidden Markov models. In a GAN, two neural networks compete against each other, with one generating samples and the other evaluating whether they are real or generated. A hidden Markov model is a generative model that has been successful in various sequence labeling tasks such as chunking, named entity recognition, POS tagging, and speech recognition.

A deep neural network model may include an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

In some embodiments, a model is, or includes, a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

In some embodiments, a model is, or includes, a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes'theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

In some embodiments, a model is, or includes, a Boltzmann machine. A Boltzmann machine comprises a set of binary units that are connected through weighted connections. Boltzmann Machines may use directionless unsupervised generative deep learning network for recommended systems.

In some embodiments, a model is, or includes, a nearest neighbor algorithm. Nearest neighbor models can be memory-based and include no model to be fit. For nearest neighbors, given a query point x0 (a test subject), the k training points x(r), r, . . . k (here the training subjects) closest in distance to x0 are identified and then the point x0 is classified using the k nearest neighbors. Here, the distance to these neighbors is a function of the abundance values of the discriminating gene set. In some embodiments, Euclidean distance in feature space is used to determine distance as Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

As an example, a k-nearest neighbor model is a non-parametric machine learning method in which the input includes the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

In some embodiments, a model is, or includes, a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

In some embodiments, a model uses a regression algorithm. A regression algorithm can be any type of regression. For example, the regression algorithm may be logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as a model (e.g., a linear model) in some embodiments of the present disclosure.

In some embodiments, a model is a mixture model, such as that described in McLachlan et al., Bioinformatics 18(3):413-422, 2002. In some embodiments, in particular, those embodiments including a temporal component, a model is a hidden Markov model such as described by Schliep et al., 2003, Bioinformatics 19(1):i255-i263.

In some embodiments, a model is an unsupervised clustering model. In some embodiments, a model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

In some embodiments, an ensemble (e.g., two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

In some embodiments, a model is a reinforcement learning model. In some embodiments, the reinforcement learning system comprises four main elements-an agent, a policy, a reward signal, and a value function, where the behavior of the agent is defined in terms of the policy. In some embodiments, the reinforcement learning system comprises a learning algorithm. In some implementations, the learning algorithm is an on-policy learning algorithm or an off-policy learning algorithm. On-Policy learning algorithms evaluate and improve the same policy which is being used to select the agent's actions. Off-Policy learning algorithms evaluate and improve policies that are different from the policy being used for action selection. Reinforcement learning is further described, for example, in Sutton RS, Barto AG, “Reinforcement learning: an introduction,” IEEE Transactions on Neural Networks. 1998; 9(5):1054-1054, which is hereby incorporated herein by reference in its entirety.

In some embodiments, a model is, or includes, an autoencoder. An autoencoder is a type of generative model used for unsupervised learning that learns a latent representation of the image and uses that to reconstruct the image. The autoencoder may be a variational autoencoder (VAE) that learns to generate new data samples that are similar to a training dataset.

In some embodiments, a model is, or includes, a transformer model. As described previously, a transformer model is a neural network that learns context and thus meaning by tracking relationships in sequential data like the words in this sentence. Transformer models are used to generate images and audio as well as text.

In some embodiments, a model is, or includes, a diffusion model. A diffusion model generates data points that are similar to the data points on which the model has been trained. In some embodiments, a model is, or includes, a probabilistic generative model, such as a Bayesian network in which the joint distribution between all of the model variables can be expressed as a function of their parents.

As used herein, the term “instruction” refers to an order given to a computer processor by a computer program. On a digital computer, in some embodiments, each instruction is a sequence of 0's and 1's that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal Instruction Set Computers (MISC), Very Long Instruction Word (VLIW), Explicitly Parallel Instruction Computing (EPIC), and One Instruction Set Computer (OISC).

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. As such, the algorithms, models, regressors, and/or classifiers of the present disclosure cannot be mentally performed. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

In some embodiments, the methods described herein include inputting information into a model comprising a plurality of parameters, where the model applies the plurality parameters to the information through a plurality of instructions to generate an output from the model.

In some embodiments, an algorithm, model, regressor, and/or classifier of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n ≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×106, n≥5×106, or n≥1×107. In some embodiments n is between 10,000 and 1×107, between 100,000 and 5×106, or between 500,000 and 1×106. In some embodiments, the plurality of parameters is at least 1000 parameters, at least 5000 parameters, at least 10,000 parameters is at least 50,000 parameters, at least 100,000 parameters, at least 250,000 parameters, at least 500,000 parameters, at least 1 million parameters, at least 5 million parameters, at least 10 million parameters, at least 25 million parameters, at least 50 million parameters, at least 100 million parameters, at least 250 million parameters, at least 500 million parameters, at least 1 billion parameters, or more parameters.

In some embodiments, the plurality of instructions is at least 1000 instructions, at least 5000 instructions, at least 10,000 instructions is at least 50,000 instructions, at least 100,000 instructions, at least 250,000 instructions, at least 500,000 instructions, at least 1 million instructions, at least 5 million instructions, at least 10 million instructions, at least 25 million instructions, at least 50 million instructions, at least 100 million instructions, at least 250 million instructions, at least 500 million instructions, at least 1 billion instructions, or more instructions.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “set” refers to a group of one or more objects. As used herein, the terms “request,” “prompt,” and “query” are used interchangeable unless expressly stated otherwise. As used herein, the term “model” refers to a machine learning model or algorithm. In some embodiments, the model is a task-specific model (e.g., a task-specific machine-learning model). As used herein, the term “task-specific” refers to a component that is specifically configured to perform a single task or a subset of tasks (e.g., a single class of tasks). In some embodiments, the model is an unsupervised learning algorithm. One example of an unsupervised learning algorithm is cluster analysis.

As used herein, the term “if” can be construed to mean “when” or “upon” or “in response to determining” or “in accordance with a determination” or “in response to detecting” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purposes of explanation, has been described with reference to specific embodiments. However, the illustrative discussions above are not intended to be exhaustive or limit the claims to the precise forms disclosed. Many modifications and variations are possible in view of the above teachings. The embodiments were chosen and described in order to best explain principles of operation and practical applications, to thereby enable others skilled in the art.

1. A method of generating inferences from multi-modal data, the method comprising:
obtaining a set of data items comprising a plurality of modalities, the set of data items including a first plurality of data items of a first modality and a second plurality of data items of a second modality;
generating, using one or more machine-learning (ML) models, summary data for the set of data items, the summary data including:
a first type of summary data for the first plurality of data items, and
a second type of summary data for the second plurality of data items;
generating a set of multi-modal embeddings using the first type of summary data and the second type of summary data;
providing the set of multi-modal embeddings to a multi-modal ML model, the multi-modal ML model being distinct from the one more ML models;
providing information from a user request to the multi-modal ML model;
receiving an output from the multi-modal ML model that is based on the information from the user request and the set of multi-modal embeddings; and
generating a response for a user using the output from the multi-modal ML model.

2. The method of claim 1, wherein the one or more ML models are components of a set of task-specific orchestrations.

3. The method of claim 1, wherein the first modality or the second modality comprises text, and wherein the first type of summary data or the second type of summary data comprises a summarization of the text.

4. The method of claim 1, wherein the first modality or the second modality comprises images, and wherein the first type of summary data or the second type of summary data comprises edited images.

5. The method of claim 1, wherein the first modality or the second modality comprises a pathology slide image, and wherein the first type of summary data or the second type of summary data comprises an annotated version of the pathology slide image.

6. The method of claim 1, wherein:
the set of data items correspond to an electronic health record for a cancer subject;
the method further comprises generating a de-identified health record for the cancer subject by de-identifying the electronic health record;
the summary data is generated using the de-identified health record and comprises a chronological account of medical events involving the cancer subject;
the user request comprises a request to calculate overall survivorship (OS) for the cancer subject; and
the output from the multi-modal ML model indicates the OS for the cancer subject.

7. The method of claim 1, further comprising:
obtaining a third plurality of data items of a third modality;
generating a third type of summary data for the third plurality of data items; and
wherein the set of multi-modal embeddings is generated using the first type of summary data, the second type of summary data, and the third type of summary data.

8. The method of claim 1, further comprising:
generating a first set of embeddings from the first type of summary data; and
generating a second set of embeddings from the second type of summary data, wherein the set of multi-modal embeddings are generated by aggregating the first and second sets of embeddings.

9. The method of claim 1, wherein the multi-modal ML model is a component of task-specific orchestration.

10. The method of claim 1, further comprising:
selecting, from the one or more ML models, a first ML model for generating the first type of summary data, wherein the first ML model is selected based on the first modality; and
selecting, from the one or more ML models, a second ML model for generating the second type of summary data, wherein the second ML model is selected based on the second modality.

11. The method of claim 1, wherein generating the set of multi-modal embeddings comprises incorporating default data into the set of multi-modal embeddings in accordance with a determination that the set of data items is missing data.

12. The method of claim 1, wherein the plurality of modalities comprises one or more of: a structured text modality, an unstructured text modality, a tabular data modality, a data visualizations modality, an image modality, an audio modality, a video modality, a biological sequence modality, a natural language modality, and a source code modality.

13. The method of claim 1, wherein the set of multi-modal embeddings are generated using a set of ML models.

14. The method of claim 1, wherein the response for the user comprises an indication of which data modalities from the plurality of modalities were used to generate the response.

15. The method of claim 1, wherein the response for the user includes an indication of what source data was used to generate the output from the multi-modal ML model.

16. The method of claim 1, further comprising:
determining which modalities of the plurality of modalities were used to generate the output from the multi-modal ML model;
based on the determined modalities, sending a request to a second ML model to generate an output responsive to the user request, wherein the second ML model is different than the one or more ML models and the multi-modal ML model; and
receiving, from the second ML model, an additional output responsive to the user request, wherein the response for the user is generated based on the additional output.

17. The method of claim 1, further comprising:
identifying an output type for the output from the multi-modal ML model;
identifying one or more criteria for the output based on the identified output type; and
determining whether the output from the multi-modal ML model meets the one or more criteria;
wherein the response for the user is generated in accordance with a determination that the output from the multi-modal ML model meets the one or more criteria.

18. The method of claim 1, wherein:
the set of data items comprise imaging data corresponding to one or more tests performed on a subject;
the summary data comprises a characterization of the imaging data;
the user request comprises a request to identify a disease state based on the set of data items; and
the output from the multi-modal ML model indicates an identified disease state.

19. A non-transitory computer-readable storage medium including instructions that, when executed by one or more processors, cause the one or more processors to perform operations including:
obtaining a set of data items comprising a plurality of modalities, the set of data items including a first plurality of data items of a first modality and a second plurality of data items of a second modality;
generating, using one or more machine-learning (ML) models, summary data for the set of data items, the summary data including:
a first type of summary data for the first plurality of data items, and
a second type of summary data for the second plurality of data items;
generating a set of multi-modal embeddings using the first type of summary data and the second type of summary data;
providing the set of multi-modal embeddings to a multi-modal ML model, the multi-modal ML model being distinct from the one more ML models;
providing information from a user request to the multi-modal ML model;
receiving an output from the multi-modal ML model that is based on the information from the user request and the set of multi-modal embeddings; and
generating a response for the user using the output from the multi-modal ML model.

20. A computing system, comprising:
one or more processors;
memory; and
one or more programs, wherein the one or more programs are stored in the memory and configured to be executed by the one or more processors, the one or more programs including instructions for:
obtaining a set of data items comprising a plurality of modalities, the set of data items including a first plurality of data items of a first modality and a second plurality of data items of a second modality;
generating, using one or more machine-learning (ML) models, summary data for the set of data items, the summary data including:
a first type of summary data for the first plurality of data items, and
a second type of summary data for the second plurality of data items;
generating a set of multi-modal embeddings using the first type of summary data and the second type of summary data;
providing the set of multi-modal embeddings to a multi-modal ML model, the multi-modal ML model being distinct from the one more ML models;
providing information from a user request to the multi-modal ML model;
receiving an output from the multi-modal ML model that is based on the information from the user request and the set of multi-modal embeddings; and
generating a response for the user using the output from the multi-modal ML model.
